SEQUENCE LISTING (43 sequences)

SEQ ID NO: 1 - DNA, 4029 bp, Escherichia coli
atggtttactcctataccgagaaaaaacgtattcgtaaggattttggtaaacgtccacaagttctggatgtaccttatctcctttctatccagcttgactcgtttcagaaatttatcgagcaagatcctgaagggcagtatggtctggaagctgctttccgttccgtattcccgattcagagctacagcggtaattccgagctgcaatacgtcagctaccgccttggcgaaccggtgtttgacgtccaggaatgtcaaatccgtggcgtgacctattccgcaccgctgcgcgttaaactgcgtctggtgatctatgagcgcgaagcgccggaaggcaccgtaaaagacattaaagaacaagaagtctacatgggcgaaattccgctcatgacagacaacggtacctttgttatcaacggtactgagcgtgttatcgtttcccagctgcaccgtagtccgggcgtcttctttgactccgacaaaggtaaaacccactcttcgggtaaagtgctgtataacgcgcgtatcatcccttaccgtggttcctggctggacttcgaattcgatccgaaggacaacctgttcgtacgtatcgaccgtcgccgtaaactgcctgcgaccatcattctgcgcgccctgaactacaccacagagcagatcctcgacctgttctttgaaaaagttatctttgaaatccgtgataacaagctgcagatggaactggtgccggaacgcctgcgtggtgaaaccgcatcttttgacatcgaagctaacggtaaagtgtacgtagaaaaaggccgccgtatcactgcgcgccacattcgccagctggaaaaagacgacgtcaaactgatcgaagtcccggttgagtacatcgcaggtaaagtggttgctaaagactatattgatgagtctaccggcgagctgatctgcgcagcgaacatggagctgagcctggatctgctggctaagctgagccagtctggtcacaagcgtatcgaaacgctgttcaccaacgatctggatcacggcccatatatctctgaaaccttacgtgtcgacccaactaacgaccgtctgagcgcactggtagaaatctaccgcatgatgcgccctggcgagccgccgactcgtgaagcagctgaaagcctgttcgagaacctgttcttctccgaagaccgttatgacttgtctgcggttggtcgtatgaagttcaaccgttctctgctgcgcgaagaaatcgaaggttccggtatcctgagcaaagacgacatcattgatgttatgaaaaagctcatcgatatccgtaacggtaaaggcgaagtcgatgatatcgaccacctcggcaaccgtcgtatccgttccgttggcgaaatggcggaaaaccagttccgcgttggcctggtacgtgtagagcgtgcggtgaaagagcgtctgtctctgggcgatctggataccctgatgccacaggatatgatcaacgccaagccgatttccgcagcagtgaaagagttcttcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgaatacggcttccttgagactccgtatcgtaaagtgaccgacggtgttgtaactgacgaaattcactacctgtctgctatcgaagaaggcaactacgttatcgcccaggcgaactccaacttggatgaagaaggccacttcgtagaagacctggtaacttgccgtagcaaaggcgaatccagcttgttcagccgcgaccaggttgactacatggacgtatccacccagcaggtggtatccgtcggtgcgtccctgatcccgttcctggaacacgatgacgccaaccgtgcattgatgggtgcgaacatgcaacgtcaggccgttccgactctgcgcgctgataagccgctggttggtactggtatggaacgtgctgttgccgttgactccggtgtaactgcggtagctaaacgtggtggtgtcgttcagtacgtggatgcttcccgtatcgttatcaaagttaacgaagacgagatgtatccgggtgaagcaggtatcgacatctacaacctgaccaaatacacccgttctaaccagaacacctgtatcaaccagatgccgtgtgtgtctctgggtgaaccggttgaacgtggcgacgtgctggcagacggtccgtccaccgacctcggtgaactggcgcttggtcagaacatgcgcgtagcgttcatgccgtggaatggttacaacttcgaagactccatcctcgtatccgagcgtgttgttcaggaagaccgtttcaccaccatccacattcaggaactggcgtgtgtgtcccgtgacaccaagctgggtccggaagagatcaccgctgacatcccgaacgtgggtgaagctgcgctctccaaactggatgaatccggtatcgtttacattggtgcggaagtgaccggtggcgacattctggttggtaaggtaacgccgaaaggtgaaactcagctgaccccagaagaaaaactgctgcgtgcgatcttcggtgagaaagcctctgacgttaaagactcttctctgcgcgtaccaaacggtgtatccggtacggttatcgacgttcaggtctttactcgcgatggcgtagaaaaagacaaacgtgcgctggaaatcgaagaaatgcagctcaaacaggcgaagaaagacctgtctgaagaactgcagatcctcgaagcgggtctgttcagccgtatccgtgctgtgctggtagccggtggcgttgaagctgagaagctcgacaaactgccgcgcgatcgctggctggagctgggcctgacagacgaagagaaacaaaatcagctggaacagctggctgagcagtatgacgaactgaaacacgagttcgagaagaaactcgaagcgaaacgccgcaaaatcacccagggcgacgatctggcaccgggcgtgctgaagattgttaaggtatatctggcggttaaacgccgtatccagcctggtgacaagatggcaggtcgtcacggtaacaagggtgtaatttctaagatcaacccgatcgaagatatgccttacgatgaaaacggtacgccggtagacatcgtactgaacccgctgggcgtaccgtctcgtatgaacatcggtcagatcctcgaaacccacctgggtatggctgcgaaaggtatcggcgacaagatcaacgccatgctgaaacagcagcaagaagtcgcgaaactgcgcgaattcatccagcgtgcgtacgatctgggcgctgacgttcgtcagaaagttgacctgagtaccttcagcgatgaagaagttatgcgtctggctgaaaacctgcgcaaaggtatgccaatcgcaacgccggtgttcgacggtgcgaaagaagcagaaattaaagagctgctgaaacttggcgacctgccgacttccggtcagatccgcctgtacgatggtcgcactggtgaacagttcgagcgtccggtaaccgttggttacatgtacatgctgaaactgaaccacctggtcgacgacaagatgcacgcgcgttccaccggttcttacagcctggttactcagcagccgctgggtggtaaggcacagttcggtggtcagcgtttcggggagatggaagtgtgggcgctggaagcatacggcgcagcatacaccctgcaggaaatgctcaccgttaagtctgatgacgtgaacggtcgtaccaagatgtataaaaacatcgtggacggcaaccatcagatggagccgggcatgccagaatccttcaacgtattgttgaaagagattcgttcgctgggtatcaacatcgaactggaagacgagtaa

SEQ ID NO: 2 - protein, 1342 aa, Escherichia coli [sequence omitted]

SEQ ID NO: 3 - DNA, 268 bp, Escherichia coli
gccgatttccgcagcagtgaaagagttcttcggttccagccagctgtctcagtttatggaccagaacaacccgctgtctgagattacgcacaaacgtcgtatctccgcactcggcccaggcggtctgacccgtgaacgtgcaggcttcgaagttcgagacgtacacccgactcactacggtcgcgtatgtccaatcgaaacccctgaaggtccgaacatcggtctgatcaactctctgtccgtgtacgcacagactaacgaatacggc

SEQ ID NO: 4 - DNA, 25 bp, Escherichia coli
tggaccagaacaacccgctgtctga

SEQ ID NO: 5 - DNA, 24 bp, Escherichia coli
tgcgtaatctcagacagcgggttg

SEQ ID NO: 6 - DNA, 27 bp, Escherichia coli
ttgttctggtccataaactgagacagc

SEQ ID NO: 7 - DNA, 24 bp, Escherichia coli
gcacaaacgtcgtatctccgcact

SEQ ID NO: 8 - DNA, 23 bp, Escherichia coli
cgtcgtatctccgcactcggccc

SEQ ID NO: 9 - DNA, 23 bp, Zea mays
gtataatatgatggcatgccctc

SEQ ID NO: 10 - DNA, 20 bp, Zea mays
gccggccagcatttgaaaca

SEQ ID NO: 11 - DNA, 29 bp, Artificial Sequence
  synthetic sequence
ttgacagctagctcagtcctaggtataat

SEQ ID NO: 12 - DNA, 19 bp, Artificial Sequence
  synthetic sequence
aatttctactcttgtagat

SEQ ID NO: 13 - DNA, 83 bp, Artificial Sequence
  synthetic sequence
gttttagagctagaaatagcaagttaaaataaggctagtccgttatcaacttgaaaaagtggcaccgagtcggtgcttttttt

SEQ ID NO: 14 - DNA, 3681 bp, Artificial Sequence
  synthetic sequence
agcaaactggaaaaattcaccaacctgtactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgtatcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatctttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgatctgcacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaagcgccgaaaaacatcttcaaaatcaacaccgaagtgcgtgttctgctgaaacacgatgataacccgtacgttattggcatcgaccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatcgtggaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgagcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgccgctgaccagcgcgtataaagaactgttcaacaaatacggcattaactaccagcagggcgacattcgtgcgctgctgctggaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaaacac

SEQ ID NO: 15 - DNA, 33 bp, Artificial Sequence
  synthetic sequence
atgggtagcaaaaagaggcgtatcaagcaggac

SEQ ID NO: 16 - DNA, 34 bp, Artificial Sequence
  synthetic sequence
ggatctaagaagcgtaggatcaagcaagattgag

SEQ ID NO: 17 - DNA, 33 bp, Artificial Sequence
  Synthetic sequence
atgggcagcagccatcatcaccaccatcaccat

SEQ ID NO: 18 - DNA, 96 bp, Artificial Sequence
  synthetic sequence
ttgacaattaatcatcggctcgtataatgtgtggaattgtgagcggataacaatttctagaaataattttgtttaactttaagaaggagatatacc

SEQ ID NO: 19 - DNA, 3684 bp, Artificial Sequence
  synthetic sequence
agcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgcccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggcagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgca

SEQ ID NO: 20 - DNA, 4101 bp, Streptococcus pyogenes
gataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 21 - DNA, 4101 bp, Artificial Sequence
  synthetic sequence
gataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgac

SEQ ID NO: 22 - protein, 1368 aa, Artificial sequence
  Synthetic sequence
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 23 - protein, 1368 aa, Streptococcus pyogenes [sequence omitted]

SEQ ID NO: 24 - protein, 1229 aa, Artificial sequence
  Synthetic sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHA

SEQ ID NO: 25 - protein, 1228 aa, Artificial sequence
  Synthetic sequence
MSKLEKFTNLYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRIINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKSLQKIDKDDVNGNYEKINYKLLPGPNKMLPKVFFSKKWMAYYNPSEDIQKIYKNGTFKKGDMFNLNDLHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKAPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKIVELVEKYDAVIALEDLNSGFKNSRVKVEKQVYQKFEKMLIDKLNYMVDKKSNPSATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVPLTSAYKELFNKYGINYQQGDIRALLLEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 26 - DNA, 491 bp, Artificial Sequence
  Synthetic sequence
tggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataagcttagtctatttagtataatatgatggcatgccctcttaggcagtagccggccagcatttgaaacatggtacttcgttttatttaaaaaataataagttattatttattatttatttgttttatcatttaaggtagtttaagcttggctgttttggcggatgagagaagattttcagcctgatacagattaaatcagaacgcagaagcggtctgataaaacagaatttgcctggcg

SEQ ID NO: 27 - DNA, 3684 bp, Artificial Sequence
  Synthetic sequence.
agcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgaccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgca

SEQ ID NO: 28 - RNA, 44 nt, Artificial Sequence
  Synthetic sequence
guaauuucuacucuuguagauguauaauaugauggcaugcccuc

SEQ ID NO: 29 - DNA, 630 bp, Bacillus subtilis
atgggaaaggtttatgtatttgatcatcctttaattcagcacaagctgacatatatacggaatgaaaatacaggtacgaaggattttagagagttagtagatgaagtggctacactcatggcatttgaaattacccgcgatcttcctctggaagaagtggatatcaatacaccggttcaggctgcgaaatcgaaagtcatctcagggaaaaaactcggagtggttcctatcctcagagcaggattgggaatggttgacggcattttaaagctgattcctgcggcaaaagtgggacatgtcggcctttaccgtgatccagaaaccttaaaacccgtggaatactatgtcaagcttccttctgatgtggaagagcgtgaattcatcgtggttgacccgatgctcgctacaggcggttccgcagttgaagccattcacagccttaaaaaacgcggtgcgaaaaatatccgtttcatgtgtcttgtagcagcgccggagggtgtggaagaattgcagaagcatcattcggacgttgatatttacattgcggcgctagatgaaaaattaaatgaaaaaggatatattgttccaggtctcggagatgcgggtgaccgcatgtttggaacaaaataa

SEQ ID NO: 30 - DNA, 1980 bp, Bacillus subtilis
atgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcgatgggatgtcacgcagaattcattgctcgggctgtatgactggaatacacaaaatacacaagtacagtcctatctgaaacggttcttagacagggcattgaatgacggggcagacggttttcgatttgatgccgccaaacatatagagcttccagatgatggcagttacggcagtcaattttggccgaatatcacaaatacatctgcagagttccaatacggagaaatcctgcaggatagtgcctccagagatgctgcatatgcgaattatatggatgtgacagcgtctaactatgggcattccataaggtccgctttaaagaatcgtaatctgggcgtgtcgaatatctcccactatgcatctgatgtgtctgcggacaagctagtgacatgggtagagtcgcatgatacgtatgccaatgatgatgaagagtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagagacggcgtttaaggatggagatcaattcacaatcggaaaaggagatccatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattga

SEQ ID NO: 31 - DNA, 23 bp, Bacillus subtilis
agataggactgtacttgtgtatt

SEQ ID NO: 32 - DNA, 23 bp, Bacillus subtilis
cccgggaacctcacaccatttcc

SEQ ID NO: 33 - DNA, 23 bp, Bacillus subtilis
cctggctccaatgattcggattt

SEQ ID NO: 34 - DNA, 23 bp, Bacillus subtilis
gcggcatcaaatcgaaaaccgtc

SEQ ID NO: 35 - DNA, 23 bp, Bacillus subtilis
gaatactcttaacctcattggaa

SEQ ID NO: 36 - DNA, 19 bp, Bacillus subtilis
agcgccgcaatgtaaatat

SEQ ID NO: 37 - DNA, 19 bp, Bacillus subtilis
cctgaaccggtgtattgat

SEQ ID NO: 38 - DNA, 19 bp, Bacillus subtilis
gccgtcaaccattcccaat

SEQ ID NO: 39 - DNA, 19 bp, Bacillus subtilis
cgtatatatgtcagcttgt

SEQ ID NO: 40 - DNA, 19 bp, Bacillus subtilis
gccatgagtgtagccactt

SEQ ID NO: 41 - DNA, 418 bp, Artificial Sequence
  Synthetic sequence. Expression cassette for guide array targeting upp gene of B. subtilis
cctccttgacactgaatttagcatgtgatataattaacttaatattctacccaagcttataaaagagcactgttgggcgtgagtggaggcgccggaaaaaagcatcgaaaaaaaatttctactaagtgtagatatctagcgccgcaatgtaaatataatttctactaagtgtagatgcagcctgaaccggtgtattgataatttctactaagtgtagataaatgccgtcaaccattcccaataatttctactaagtgtagatattccgtatatatgtcagcttgtaatttctactaagtgtagataaatgccatgagtgtagccacttaatttctactaagtgtagatttttcaattgcatgaagaatctgcttacataaccccttggggcctctaaacgggtcttgaggggtttttt

SEQ ID NO: 42 - DNA, 418 bp, Artificial Sequence
  Synthetic sequence. Expression cassette for guide array targeting amyE gene of B. subtilis
cctccttgacactgaatttagcatgtgatataattaacttaatattctacccaagcttataaaagagcactgttgggcgtgagtggaggcgccggaaaaaagcatcgaaaaaaaatttctactaagtgtagatagataggactgtacttgtgtattaatttctactaagtgtagatcccgggaacctcacaccatttccaatttctactaagtgtagatcctggctccaatgattcggatttaatttctactaagtgtagatgcggcatcaaatcgaaaaccgtcaatttctactaagtgtagatgaatactcttaacctcattggaaaatttctactaagtgtagatttttcaattgcatgaagaatctgcttacataaccccttggggcctctaaacgggtcttgaggggtttttt

SEQ ID NO: 43 - DNA, 3953 bp, Artificial Sequence
  Synthetic sequence. Expression cassette for dLbCpf1 with N- and C-terminal NLS sequences
ggcgcgccctaatttgacagtagaattagcatgtgatataataaataatttttactacccaagcttataaaagagcactgttgggcgtgagtggaggcgccggaaaaaagcatcgaaaaaaggaggaaaaaaacatatgggtagcaaaaagaggcgtatcaagcaggacagcaaactggaaaaattcaccaactgttactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgttgcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatgtttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgattgccacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaatgcccgaaaaacatcttcaaaatcaacaccgaagtgcgtgttctgctgaaacacgatgataacccgtacgttattggcatcgcccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatctgtgaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgtgcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgtgcctgaccagcgcgtataaagaactgttcaacaaatacggcattaactaccagcagggcgacattcgtgcgctgctgtgtgaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaagcatgcaggatctaagaagcgtaggatcaagcaagattgagcggccgctgagcaataactagcataaccccttggggcctctaaacgggtcttgaggggttttttgc

(19) United States
(12) Patent Application Publication
(10) Pub. No.: US 2020/0347389 A1
(43) Pub. Date: Nov. 5, 2020

(54) COMPOSITIONS AND METHODS FOR GENERATING DIVERSITY AT TARGETED NUCLEIC ACID SEQUENCES

(71) Applicant: MONSANTO TECHNOLOGY LLC, St. Louis, MO (US)

(72) Inventors: Graeme S. GARVEY, Woodland, CA (US); Farhad MOSHIRI, Chesterfield, MO (US)

(73) Assignee: MONSANTO TECHNOLOGY LLC, St. Louis, MO (US)

(21) Appl. No.: 16/865,129

(22) Filed: May 1, 2020

Related U.S. Application Data
(60) Provisional application No. 62/842,184, filed on May 2, 2019.

(51) Int. Cl. C12N 15/113; C12N 9/22

(52) U.S. Cl. CPC C12N 15/113; C12N 9/22

(57) ABSTRACT

The present disclosure provides methods and kits useful for generating targeted modifications in target nucleic acids using catalytically inactive guided-nucleases in combination with mutagens.

### CROSS-REFERENCE TO RELATED APPLICATION

This application claims the benefit of U.S. Provisional Application No. 62/842,184, filed May 2, 2019, which is incorporated by reference in its entirety herein.

### FIELD

The present disclosure relates to compositions and methods related to using catalytically inactive guided-nucleases in combination with mutagens to generate modifications at targeted nucleic acids.

### INCORPORATION OF SEQUENCE LISTING

A sequence listing contained in the file named P34716US01_SEQ.txt, which is 104,213 bytes (measured in MS-Windows®) and created on May 1, 2020, and comprises 43 sequences, is filed electronically herewith and incorporated by reference in its entirety.

### BACKGROUND

Chemical mutagens, such as ethyl methanesulfonate (EMS), and ionizing radiation have long been used as a tool in plant breeding to induce genetic variation. Plant varieties with desirable traits such as larger seed size, disease resistance, and better fiber quality have been developed via mutagenesis. The genetic changes introduced by mutagens occurs randomly within the genome, providing the breeder with no precise control over the number of mutations, their location in the genome, or the cells targeted by the mutagen (e.g., somatic vs. germ cell). A breeder may adjust the dosage of a mutagen to limit or maximize the number of mutations introduced. A high dose of the mutagen may be used to induce a high rate of mutation, increasing the probability of a mutation occurring in a desired target, however, high mutation rates also result in high mortality. Lower doses of the mutagen will result in a higher rate of survival and fewer mutations within the genome, thus decreasing the likelihood that a mutation will occur in a desired target and necessitating the screening of large numbers of plants to identify valuable mutations. The inability to target mutagenic activity to chosen regions of a genome requires breeders to expend resources in exposing large number of plants to a mutagen and in screening large numbers mutagenized plants in order to identify and recover a mutation that has occurred randomly in a desired location. It would therefore be desirable to focus the mutagenic activity of a mutagen to a targeted region within the genome.

### BRIEF DESCRIPTION OF DRAWINGS

FIG. 1 depicts the Rif^{r} mutations and their frequency of occurrence within the rpoB gene induced buy the chemical mutagen, EMS, in the absence of targeted dCas9. Rif^{r} colonies were generated from cells expressing dCas9 and gRNA targeting Zm7.1 (a sequence not found in the E. coli genome) treated to 0.1% or 1% EMS. Only positions which had a mutation within SEQ ID 3 which is a fragment of the rpoB gene (SEQ ID NO:1) are shown. ‘Position’=nucleotide position in SEQ ID NO:1.

FIG. 2 depicts a summary of rpoB mutations and their frequency of occurrence induced by the chemical mutagen, EMA, in combination with targeted and non-targeted catalytically inactive RNA-guide nucleases. Only positions which had a mutation within SEQ ID NO:3 which is a fragment of the rpoB gene (SEQ ID NO:1) are shown. Rif^{r} colonies where generated from cells expressing dCpf1 or dCas9 and their cognate gRNAs treated with 0.1% EMS. ‘Cas9-TS’=the nucleotides residing within the Sp-rpoB-1526 Cas9 gRNA target site. ‘Cpf1-TS’=the nucleotides residing within the Sp-rpoB-1578 Cpf1 gRNA target site. ‘WT seq’=SEQ ID NO:1. ‘Position’=position of the nucleotide with respect to SEQ ID NO:1. G and C residues within WT seq serve as potential targets for EMS-induced transitions are shaded in gray. ‘-’=in-frame/3n deletions. ‘CAT’ at position 1590=in-frame insertion. A* at position 1530=silent mutation.

FIG. 3 depicts the frequency of double mutations as detected by PCR in colonies from EMS treated E. coli cells expressing dCas9+g-rpoB-1526; dCas9+g-Zm7.1; or dCpf1+g-rpoB-1578. Count=number of colonies.

FIG. 4 shows a plot of the rate of 5-FU resistant CFU relative to total CFU for each tested plasmid combination, in both cycled illumination (light) and dark conditions.

### SUMMARY

In one aspect, this disclosure provides a method of introducing a modification in a target nucleic acid molecule, comprising contacting the target nucleic acid molecule with: (a) a catalytically inactive guided-nuclease; and (b) at least one mutagen, where at least one modification is introduced in the target nucleic acid molecule. In some embodiments, the target nucleic acid molecule is further contacted by at least one guide nucleic acid, wherein the at least one guide nucleic acid forms a complex with the catalytically inactive guided-nuclease, and wherein the at least one guide nucleic acid hybridizes with the target nucleic acid molecule. In some embodiments, the catalytically inactive guided-nuclease interacts with the target nucleic acid molecule directly through a DNA-binding domain. In some embodiments the target nucleic acid molecule is contacted in vitro. In some embodiments the target nucleic acid molecule is contacted in vivo. In some embodiments the target nucleic acid molecule is in the genome of a prokaryotic cell. In some embodiments, the prokaryotic cell is selected from an Escherichia coli cell, a Bacillus subtilis cell, a Bacillus thuringiensis cell, a Bacillus coagulans cell, a Thermus aquaticus cell, and a Pseudomonas chlororaphis cell. In some embodiments the target nucleic acid molecule is in the genome of a eukaryotic cell. In some embodiments, the eukaryotic cell is selected from a plant cell, a non-human animal cell, a human cell, an algae cell, and a yeast cell. In some embodiments, the plant cell is selected from the group consisting of: a corn cell, a cotton cell, a canola cell, a soybean cell, a barley cell, a rye cell, a rice cell, a tomato cell, a pepper cell, a wheat cell, an alfalfa cell, a sorghum cell, an Arabidopsis cell, a cucumber cell, a potato cell, a sweet potato cell, a carrot cell, an apple cell, a banana cell, a pineapple cell, a blueberry cell, a blackberry cell, a raspberry cell, a strawberry cell, a cucurbit cell, a brassica cell, a citrus cell, and an onion cell. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light. In some embodiments, the catalytically inactive guided-nuclease is a catalytically inactive CRISPR nuclease. In some embodiments, the catalytically inactive CRISPR nuclease is selected from the group consisting of a catalytically inactive Cas9, a catalytically inactive Cpf1, a catalytically inactive CasX, a catalytically inactive CasY, a catalytically inactive C2c2, a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, and a catalytically inactive Csf4. In some embodiments, the target nucleic acid molecule comprises a protospacer adjacent motif (PAM). In some embodiments, the target nucleic acid molecule comprises a nucleotide sequence, from 5′ to 3′, selected from the group consisting of NGG, NGA, TTTN, YG, YTN, TTCN, NGAN, NGNG, NGAG, NGCG, TYCV, NGRRT, NGRRN, NNNNGATT, NNNNRYAC, NNAGAAW, and NAAAAC.

In one aspect, this disclosure provides a method of inducing a targeted modification in a target nucleic acid molecule, comprising contacting the target nucleic acid molecule with (a) a catalytically inactive guided-nuclease; (b) at least one guide nucleic acid, where the at least one guide nucleic acid forms a complex with the catalytically inactive guided-nuclease, and where the at least one guide nucleic acid hybridizes with the target nucleic acid molecule; and (c) at least one mutagen, where the target nucleic acid molecule comprises a protospacer adjacent motif (PAM) site, and where at least one modification is induced in the target nucleic acid molecule within 100 nucleotides of the PAM site. In some embodiments, at least one modification is induced in the target nucleic acid molecule within 90 nucleotides of the PAM site. In some embodiments, at least one modification is induced in the target nucleic acid molecule within 80 nucleotides of the PAM site. In some embodiments, at least one modification is induced in the target nucleic acid molecule within 70 nucleotides of the PAM site. In some embodiments, at least one modification is induced in the target nucleic acid molecule within 60 nucleotides of the PAM site. In some embodiments, at least one modification is induced in the target nucleic acid molecule within 50 nucleotides of the PAM site. In some embodiments, at least one modification is induced in the target nucleic acid molecule within 40 nucleotides of the PAM site. In some embodiments, at least one modification is induced in the target nucleic acid molecule within 30 nucleotides of the PAM site. In some embodiments, at least one modification is induced in the target nucleic acid molecule within 20 nucleotides of the PAM site. In some embodiments, at least one modification is induced in the target nucleic acid molecule within 10 nucleotides of the PAM site. In some embodiments, the PAM site comprises a nucleotide sequence, from 5′ to 3′, selected from the group consisting of NGG, NGA, TTTN, YG, YTN, TTCN, NGAN, NGNG, NGAG, NGCG, TYCV, NGRRT, NGRRN, NNNNGATT, NNNNRYAC, NNAGAAW, and NAAAAC. In some embodiments, the catalytically inactive guided-nuclease is a catalytically inactive CRISPR nuclease. In some embodiments, the catalytically inactive CRISPR nuclease is selected from the group consisting of a catalytically inactive Cas9, a catalytically inactive Cpf1, a catalytically inactive CasX, a catalytically inactive CasY, a catalytically inactive C2c2, a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, and a catalytically inactive Csf4. In some embodiments, the at least one guide nucleic acid comprises a crRNA. In some embodiments, the at least one guide nucleic acid comprises a tracrRNA. In some embodiments, the at least one guide nucleic acid comprises a single-molecule guide. In some embodiments, the at least one guide nucleic acid comprises at least 80% complementarity to a target region of the target nucleic acid molecule. In some embodiments the target nucleic acid molecule is contacted in vitro. In some embodiments the target nucleic acid molecule is contacted in vivo. In some embodiments the target nucleic acid molecule is in the genome of a prokaryotic cell. In some embodiments, the prokaryotic cell is selected from an Escherichia coli cell, a Bacillus subtilis cell, a Bacillus thuringiensis cell, a Bacillus coagulans cell, a Thermus aquaticus cell, and a Pseudomonas chlororaphis cell. In some embodiments the target nucleic acid molecule is in the genome of a eukaryotic cell. In some embodiments, the eukaryotic cell is selected from a plant cell, a non-human animal cell, a human cell, an algae cell, and a yeast cell. In some embodiments, the plant cell is selected from the group consisting of: a corn cell, a cotton cell, a canola cell, a soybean cell, a rice cell, a tomato cell, a wheat cell, an alfalfa cell, a sorghum cell, an Arabidopsis cell, a cucumber cell, a potato cell, a carrot cell, an apple cell, a banana cell, a pineapple cell, a blueberry cell, a blackberry cell, a raspberry cell, a cucurbit cell, a citrus cell, and an onion cell. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light.

In one aspect, this disclosure provides a method of increasing the mutation rate of a targeted region of a nucleic acid molecule, comprising contacting the target nucleic acid molecule with: (a) a catalytically inactive guided-nuclease; (b) at least one guide nucleic acid, where the at least one guide nucleic acid forms a complex with the catalytically inactive guided-nuclease, and where the at least one guide nucleic acid hybridizes with the nucleic acid molecule adjacent to the targeted region; and (c) at least one mutagen; where the nucleic acid molecule comprises a protospacer adjacent motif (PAM) site, and where the mutation rate in the targeted region of the nucleic acid molecule is increased as compared to an untargeted nucleic acid molecule. In some embodiments, the PAM site comprises a nucleotide sequence, from 5′ to 3′, selected from the group consisting of NGG, NGA, TTTN, YG, YTN, TTCN, NGAN, NGNG, NGAG, NGCG, TYCV, NGRRT, NGRRN, NNNNGATT, NNNNRYAC, NNAGAAW, and NAAAAC. In some embodiments, the catalytically inactive guided-nuclease is a catalytically inactive CRISPR nuclease. In some embodiments, the catalytically inactive CRISPR nuclease is selected from the group consisting of a catalytically inactive Cas9, a catalytically inactive Cpf1, a catalytically inactive CasX, a catalytically inactive CasY, a catalytically inactive C2c2, a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, and a catalytically inactive Csf4. In some embodiments, the at least one guide nucleic acid comprises a crRNA. In some embodiments, the at least one guide nucleic acid comprises a tracrRNA. In some embodiments, the at least one guide nucleic acid comprises a single-molecule guide. In some embodiments, the at least one guide nucleic acid comprises at least 80% complementarity to a target region of the target nucleic acid molecule. In some embodiments the target nucleic acid molecule is contacted in vitro. In some embodiments the target nucleic acid molecule is contacted in vivo. In some embodiments the target nucleic acid molecule is in the genome of a prokaryotic cell. In some embodiments, the prokaryotic cell is selected from an Escherichia coli cell, a Bacillus subtilis cell, a Bacillus thuringiensis cell, a Bacillus coagulans cell, a Thermus aquaticus cell, and a Pseudomonas chlororaphis cell. In some embodiments the target nucleic acid molecule is in the genome of a eukaryotic cell. In some embodiments, the eukaryotic cell is selected from a plant cell, a non-human animal cell, a human cell, an algae cell, and a yeast cell. In some embodiments, the plant cell is selected from the group consisting of: a corn cell, a cotton cell, a canola cell, a soybean cell, a rice cell, a tomato cell, a wheat cell, an alfalfa cell, a sorghum cell, an Arabidopsis cell, a cucumber cell, a potato cell, a carrot cell, an apple cell, a banana cell, a pineapple cell, a blueberry cell, a blackberry cell, a raspberry cell, a cucurbit cell, a citrus cell, and an onion cell. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light.

In one aspect, this disclosure provides a method of increasing allelic diversity in a targeted region of a nucleic acid molecule within a genome of a plant, comprising providing to the plant: (a) a catalytically inactive guided-nuclease or a nucleic acid encoding the catalytically guided-nuclease; (b) at least one guide nucleic acid or a nucleic acid encoding the at least one guide nucleic acid, where the at least one guide nucleic acid forms a complex with the catalytically inactive guided-nuclease, and where the at least one guide nucleic acid hybridizes adjacent to the targeted region of the nucleic acid molecule; and (c) at least one mutagen; where the nucleic acid comprises a protospacer adjacent motif (PAM), and where allelic diversity of the targeted region of the nucleic acid is increased. In some embodiments, the PAM is within 1-10, 10-20, 20-30, 30-40, 40-50, 50-60, 60-70, 70-80, 80-90, 90-100 nucleotides 5′ of the targeted region of the nucleic acid. In some embodiments, the PAM is 1, 2, 3, 4, 5, 6, 7, 8, 9, 10, 11, 12, 13, 14, 15, 16, 17, 18, 19, 20, 21, 22, 23, 24, 25 or more nucleotides 5′ of the targeted region of the nucleic acid. In some embodiments, the PAM is within 1-10, 10-20, 20-30, 30-40, 40-50, 50-60, 60-70, 70-80, 80-90, 90-100 nucleotides 3′ of the targeted region of the nucleic acid. In some embodiments, the PAM is 1, 2, 3, 4, 5, 6, 7, 8, 9, 10, 11, 12, 13, 14, 15, 16, 17, 18, 19, 20, 21, 22, 23, 24, 25 or more nucleotides 3′ of the targeted region of the nucleic acid. In some embodiments, the PAM site comprises a nucleotide sequence, from 5′ to 3′, selected from the group consisting of NGG, NGA, TTTN, YG, YTN, TTCN, NGAN, NGNG, NGAG, NGCG, TYCV, NGRRT, NGRRN, NNNNGATT, NNNNRYAC, NNAGAAW, and NAAAAC. In some embodiments, the catalytically inactive guided-nuclease is a catalytically inactive CRISPR nuclease. In some embodiments, the catalytically inactive CRISPR nuclease is selected from the group consisting of a catalytically inactive Cas9, a catalytically inactive Cpf1, a catalytically inactive CasX, a catalytically inactive CasY, a catalytically inactive C2c2, a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, and a catalytically inactive Csf4. In some embodiments, the at least one guide nucleic acid comprises a crRNA. In some embodiments, the at least one guide nucleic acid comprises a tracrRNA. In some embodiments, the at least one guide nucleic acid comprises a single-molecule guide. In some embodiments, the at least one guide nucleic acid comprises at least 80% complementarity to a target region of the target nucleic acid molecule. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light. In some embodiments, the plant is selected from corn, cotton, soybean, canola, wheat, barley, rice, tomato, onion, pepper, blueberry, raspberry, blackberry, strawberry, watermelon, cucurbit, brassica, spinach, eggplant, potato, sweet potato, sugar cane, oat, millet, rye, flax, alfalfa, and beet. In some embodiments, the plant comprises one or more of a nucleic acid encoding the catalytically inactive guided-nuclease and a nucleic acid encoding the at least one guide nucleic acid. In some embodiments, one or more of a nucleic acid encoding the catalytically inactive guided-nuclease and a nucleic acid encoding the at least one guide nucleic acid are provided to the plant via a method selected from the group consisting of: Agrobacterium-mediated transformation, polyethylene glycol-mediated transformation, biolistic transformation, liposome-mediated transfection, viral transduction, the use of one or more delivery particles, microinjection, and electroporation. In some embodiments, the catalytically inactive guided-nuclease and the at least one guide RNA are provided to the plant as a ribonucleoprotein. In some embodiments, the ribonucleoprotein is provided to the plant via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation. In some embodiments, allelic diversity is increased in a nucleic acid molecule encoding Brachytic1, Brachytic2, Brachytic3, Flowering Locus T, Rgh1, Rsp1, Rsp2, Rsp3, 5-Enolpyruvylshikimate-3-Phosphate Synthase (EPSPS), acetohydroxyacid synthase, dihydropteroate synthase, phytoene desaturase (PDS), Protoporphyrin IX oxygenase (PPO), para-aminobenzoate synthase, 1-deoxy-D-xylulose 5-phosphate (DOXP) synthase, dihydropteroate (DHP) synthase, phenylalanine ammonia lyase (PAL), glutathione 5-transferase (GST), D1 protein of photosystem II, mono-oxygenase, cytochrome P450, cellulose synthase, beta-tubulin, RUBISCO, translation initiation factor, phytoene desaturase double-stranded DNA adenosine tripolyphosphatase (ddATP), fatty acid desaturase 2 (FAD2), Gibberellin 20 Oxidase (GA20ox), Acetyl-CoA Carboxylase (ACC), Glutamine Synthetase (GS), p-Hydroxyphenylpyruvate Dioxygenase (HPPD), Hydroxymethyldihydropterin Pyrophosphokinase (DHPS), auxin/indole-3-acetic acid (AUX/IAA), Waxy (Wx), Acetolactate Synthase (ALS), OsERF922, OsSWEET13, OsSWEET14, TaMLO, GL2, betaine aldehyde dehydrogenase (BADH2), Matrilineal (MTL), Frigida, Grain Weight 2 (GW2), Gn1a, DEP1, GS3, SlMLO1, SlJAZ2, CsLOB1, EDR1, Self-Pruning 5G (SPSG), Slagamous-Like 6 (SlAGL6), thermosensitive genic male-sterile 5 gene (TMS5), OsMATL, ARGOS8, eukaryotic translation initiation factor 4E (eIF4E), granule-bound starch synthase (GBSS) or vacuolar invertase (VInv).

In one aspect, this disclosure provides a method of increasing allelic diversity in a targeted region of a nucleic acid molecule within a genome of a prokaryote, comprising providing to the prokaryote: (a) a catalytically inactive guided-nuclease or a nucleic acid encoding the catalytically guided-nuclease; (b) at least one guide nucleic acid or a nucleic acid encoding the at least one guide nucleic acid, where the at least one guide nucleic acid forms a complex with the catalytically inactive guided-nuclease, and where the at least one guide nucleic acid hybridizes adjacent to the targeted region of the nucleic acid molecule; and (c) at least one mutagen; where the nucleic acid comprises a protospacer adjacent motif (PAM), and where allelic diversity of the targeted region of the nucleic acid is increased. In some embodiments, the PAM is within 1-10, 10-20, 20-30, 30-40, 40-50, 50-60, 60-70, 70-80, 80-90, 90-100 nucleotides 5′ of the targeted region of the nucleic acid. In some embodiments, the PAM is 1, 2, 3, 4, 5, 6, 7, 8, 9, 10, 11, 12, 13, 14, 15, 16, 17, 18, 19, 20, 21, 22, 23, 24, 25 or more nucleotides 5′ of the targeted region of the nucleic acid. In some embodiments, the PAM is within 1-10, 10-20, 20-30, 30-40, 40-50, 50-60, 60-70, 70-80, 80-90, 90-100 nucleotides 3′ of the targeted region of the nucleic acid. In some embodiments, the PAM is 1, 2, 3, 4, 5, 6, 7, 8, 9, 10, 11, 12, 13, 14, 15, 16, 17, 18, 19, 20, 21, 22, 23, 24, 25 or more nucleotides 3′ of the targeted region of the nucleic acid. In some embodiments, the PAM site comprises a nucleotide sequence, from 5′ to 3′, selected from the group consisting of NGG, NGA, TTTN, YG, YTN, TTCN, NGAN, NGNG, NGAG, NGCG, TYCV, NGRRT, NGRRN, NNNNGATT, NNNNRYAC, NNAGAAW, and NAAAAC. In some embodiments, the catalytically inactive guided-nuclease is a catalytically inactive CRISPR nuclease. In some embodiments, the catalytically inactive CRISPR nuclease is selected from the group consisting of a catalytically inactive Cas9, a catalytically inactive Cpf1, a catalytically inactive CasX, a catalytically inactive CasY, a catalytically inactive C2c2, a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, and a catalytically inactive Csf4. In some embodiments, the at least one guide nucleic acid comprises a crRNA. In some embodiments, the at least one guide nucleic acid comprises a tracrRNA. In some embodiments, the at least one guide nucleic acid comprises a single-molecule guide. In some embodiments, the at least one guide nucleic acid comprises at least 80% complementarity to a target region of the target nucleic acid molecule. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light. In some embodiments, the prokaryote is selected from an Escherichia coli, a Bacillus subtilis, a Bacillus thuringiensis, a Bacillus coagulans, a Thermus aquaticus, and a Pseudomonas chlororaphis. In some embodiments, allelic diversity is increased in a nucleic acid encoding an insecticidal toxin.

In one aspect, this disclosure provides a method of providing a plant with an improved agronomic characteristic, comprising: (a) providing to a first plant: (i) a catalytically inactive guided-nuclease or a nucleic acid encoding the catalytically inactive guided-nuclease; (ii) at least one guide nucleic acid or a nucleic acid encoding the guide nucleic acid, where the at least one guide nucleic acid forms a complex with the catalytically inactive guided-nuclease, where the at least one guide nucleic acid hybridizes with a targeted region of a nucleic acid molecule in a genome of the plant, and wherein the nucleic acid comprises a protospacer adjacent motif (PAM) site; and (iii) at least one mutagen; where at least one modification is induced in the targeted region of the nucleic acid molecule; (b) generating at least one progeny plant from the first plant; and (c) selecting at least one progeny plant comprising the at least one modification and the improved agronomic characteristic. In some embodiments, the PAM is within 1-10, 10-20, 20-30, 30-40, 40-50, 50-60, 60-70, 70-80, 80-90, 90-100 nucleotides 5′ of the targeted region of the nucleic acid. In some embodiments, the PAM is 1, 2, 3, 4, 5, 6, 7, 8, 9, 10, 11, 12, 13, 14, 15, 16, 17, 18, 19, 20, 21, 22, 23, 24, 25 or more nucleotides 5′ of the targeted region of the nucleic acid. In some embodiments, the PAM is within 1-10, 10-20, 20-30, 30-40, 40-50, 50-60, 60-70, 70-80, 80-90, 90-100 nucleotides 3′ of the targeted region of the nucleic acid. In some embodiments, the PAM is 1, 2, 3, 4, 5, 6, 7, 8, 9, 10, 11, 12, 13, 14, 15, 16, 17, 18, 19, 20, 21, 22, 23, 24, 25 or more nucleotides 3′ of the targeted region of the nucleic acid. In some embodiments, the PAM site comprises a nucleotide sequence, from 5′ to 3′, selected from the group consisting of NGG, NGA, TTTN, YG, YTN, TTCN, NGAN, NGNG, NGAG, NGCG, TYCV, NGRRT, NGRRN, NNNNGATT, NNNNRYAC, NNAGAAW, and NAAAAC. In some embodiments, the catalytically inactive guided-nuclease is a catalytically inactive CRISPR nuclease. In some embodiments, the catalytically inactive CRISPR nuclease is selected from the group consisting of a catalytically inactive Cas9, a catalytically inactive Cpf1, a catalytically inactive CasX, a catalytically inactive CasY, a catalytically inactive C2c2, a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, and a catalytically inactive Csf4. In some embodiments, the at least one guide nucleic acid comprises a crRNA. In some embodiments, the at least one guide nucleic acid comprises a tracrRNA. In some embodiments, the at least one guide nucleic acid comprises a single-molecule guide. In some embodiments, the at least one guide nucleic acid comprises at least 80% complementarity to a target region of the target nucleic acid molecule. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light. In some embodiments, the plant is selected from corn, cotton, soybean, canola, wheat, barley, rice, tomato, onion, pepper, blueberry, raspberry, blackberry, strawberry, watermelon, cucurbit, brassica, spinach, eggplant, potato, sweet potato, sugar cane, oat, millet, rye, flax, alfalfa, and beet. In some embodiments, the plant comprises one or more of a nucleic acid encoding the catalytically inactive guided-nuclease and a nucleic acid encoding the at least one guide nucleic acid. In some embodiments, the improved agronomic characteristic is selected from the group consisting of: disease resistance, abiotic stress tolerance, insect resistance, oil content, height, drought resistance, maturity, lodging resistance, kernel weight, and yield.

In one aspect, this disclosure provides a kit for inducing a targeted modification in a target nucleic acid molecule, comprising: (a) a catalytically inactive guided-nuclease, or a nucleic acid encoding the catalytically inactive guided-nuclease; and (b) at least one chemical mutagen. In some embodiments, the catalytically inactive guided-nuclease is a CRISPR associated protein. In some embodiments, the CRISPR protein is selected from the group consisting of a catalytically inactive Cas9, a catalytically inactive Cpf1, a catalytically inactive CasX, a catalytically inactive CasY, a catalytically inactive C2c2, a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, and a catalytically inactive Csf4. In some embodiments, the kit further comprises at least one guide nucleic acid or a nucleic acid encoding the guide nucleic acid, wherein the at least one guide nucleic acid forms a complex with the catalytically inactive guided-nuclease. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the kit further comprises one or more of: a DNA-targeting nucleic acid, a reagent for reconstitution and/or dilution. In some embodiments, the kit further comprises a reagent selected from the group consisting of: a buffer for introducing the catalytically inactive guided-nuclease into cells, a wash buffer, a control reagent, a control expression vector or RNA polynucleotide, a reagent for in vitro production of the catalytically inactive guided-nuclease from DNA, a reagent for in vitro production of the DNA-targeting nucleic acid from DNA, Agrobacterium, and combinations thereof.

In one aspect, this disclosure provides a method of inducing a targeted modification in a targeted region of a nucleic acid molecule, comprising contacting the target nucleic acid molecule with: (a) a targeted DNA binding protein; and (b) at least one mutagen, where at least one modification is induced in the targeted region of the nucleic acid molecule. In some embodiments, the targeted DNA binding protein is selected from the group of: a recombinase, a helicase, a zinc finger protein, a transcription activator-like effectors (TALE), and a topoisomerase. In some embodiments, a targeted DNA binding protein has no intrinsic nucleic acid cleavage activity.

In one aspect, this disclosure provides a method of inducing a targeted modification in a targeted region of a nucleic acid molecule, comprising contacting the nucleic acid molecule with (a) a targeted DNA binding protein where the targeted DNA binding protein binds to the nucleic acid molecule; and (b) at least one mutagen, where at least one modification is induced in the targeted region of the nucleic acid molecule. In some embodiments the target nucleic acid molecule is contacted in vitro. In some embodiments the target nucleic acid molecule is contacted in vivo. In some embodiments the target nucleic acid molecule is in the genome of a prokaryotic cell. In some embodiments, the prokaryotic cell is selected from an Escherichia coli cell, a Bacillus subtilis cell, a Bacillus thuringiensis cell, a Bacillus coagulans cell, a Thermus aquaticus cell, and a Pseudomonas chlororaphis cell. In some embodiments the target nucleic acid molecule is in the genome of a eukaryotic cell. In some embodiments, the eukaryotic cell is selected from a plant cell, a non-human animal cell, a human cell, an algae cell, and a yeast cell. In some embodiments, the plant cell is selected from the group consisting of: a corn cell, a cotton cell, a canola cell, a soybean cell, a barley cell, a rye cell, a rice cell, a tomato cell, a wheat cell, an alfalfa cell, a sorghum cell, an Arabidopsis cell, a cucumber cell, a potato cell, a sweet potato cell, a pepper cell, a carrot cell, an apple cell, a banana cell, a pineapple cell, a blueberry cell, a blackberry cell, a raspberry cell, a strawberry cell, a cucurbit cell, a brassica cell, a citrus cell, and an onion cell. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light. In some embodiments, the targeted DNA binding protein is selected from the group of: a recombinase, a helicase, a zinc finger protein, a transcription activator-like effectors (TALE), and a topoisomerase. In some embodiments, a targeted DNA binding protein has no intrinsic nucleic acid cleavage activity.

In one aspect, this disclosure provides a method of increasing the mutation rate of a targeted region of a nucleic acid molecule, comprising contacting the nucleic acid molecule with: (a) a targeted DNA binding protein where the targeted DNA binding protein binds to the target nucleic acid molecule; and (b) at least one mutagen; and where the mutation rate in the targeted region of the nucleic acid molecule is increased as compared to an untargeted region of the nucleic acid molecule. In some embodiments the target nucleic acid molecule is contacted in vitro. In some embodiments the target nucleic acid molecule is contacted in vivo. In some embodiments the target nucleic acid molecule is in the genome of a prokaryotic cell. In some embodiments, the prokaryotic cell is selected from an Escherichia coli cell, a Bacillus subtilis cell, a Bacillus thuringiensis cell, a Bacillus coagulans cell, a Thermus aquaticus cell, and a Pseudomonas chlororaphis cell. In some embodiments the target nucleic acid molecule is in the genome of a eukaryotic cell. In some embodiments, the eukaryotic cell is selected from a plant cell, a non-human animal cell, a human cell, an algae cell, and a yeast cell. In some embodiments, the plant cell is selected from the group consisting of: a corn cell, a cotton cell, a canola cell, a soybean cell, a rice cell, a tomato cell, a pepper cell, a wheat cell, an alfalfa cell, a sorghum cell, an Arabidopsis cell, a cucumber cell, a potato cell, a carrot cell, an apple cell, a banana cell, a pineapple cell, a blueberry cell, a blackberry cell, a raspberry cell, a strawberry cell, a cucurbit cell, a brassica cell, a citrus cell, and an onion cell. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light. In some embodiments, the targeted DNA binding protein is selected from the group of: a recombinase, a helicase, a zinc finger protein, a transcription activator-like effectors (TALE), and a topoisomerase. In some embodiments, a targeted DNA binding protein has no intrinsic nucleic acid cleavage activity.

In one aspect, this disclosure provides a method of increasing allelic diversity in a targeted region of a nucleic acid molecule within a genome of a plant, comprising providing to the plant: (a) a targeted DNA binding protein or a nucleic acid encoding the targeted DNA binding protein where the targeted DNA binding protein binds to the target nucleic acid molecule; and (c) at least one mutagen; and where allelic diversity of the targeted region of nucleic acid is increased. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light. In some embodiments, the plant is selected from corn, cotton, soybean, canola, wheat, barley, rice, tomato, onion, pepper, blueberry, raspberry, blackberry, strawberry, watermelon, cucurbit, brassica, spinach, eggplant, potato, sweet potato, sugar cane, oat, millet, rye, flax, alfalfa, and beet. In some embodiments, the plant comprises one or more of a nucleic acid encoding the targeted DNA binding protein. In some embodiments, the nucleic acid encoding the targeted DNA binding protein are provided to the plant via a method selected from the group consisting of: Agrobacterium-mediated transformation, polyethylene glycol-mediated transformation, biolistic transformation, liposome-mediated transfection, viral transduction, the use of one or more delivery particles, microinjection, and electroporation. In some embodiments, the targeted DNA binding protein provided to the plant via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation. In some embodiments, allelic diversity is increased in a nucleic acid molecule encoding Brachytic1, Brachytic2, Brachytic3, Flowering Locus T, Rgh1, Rsp1, Rsp2, Rsp3, 5-Enolpyruvylshikimate-3-Phosphate Synthase (EPSPS), acetohydroxyacid synthase, dihydropteroate synthase, phytoene desaturase (PDS), Protoporphyrin IX oxygenase (PPO), para-aminobenzoate synthase, 1-deoxy-D-xylulose 5-phosphate (DOXP) synthase, dihydropteroate (DHP) synthase, phenylalanine ammonia lyase (PAL), glutathione 5-transferase (GST), D1 protein of photosystem II, mono-oxygenase, cytochrome P450, cellulose synthase, beta-tubulin, RUBISCO, translation initiation factor, phytoene desaturase double-stranded DNA adenosine tripolyphosphatase (ddATP), fatty acid desaturase 2 (FAD2), Gibberellin 20 Oxidase (GA20ox), Acetyl-CoA Carboxylase (ACC), Glutamine Synthetase (GS), p-Hydroxyphenylpyruvate Dioxygenase (HPPD), Hydroxymethyldihydropterin Pyrophosphokinase (DHPS), auxin/indole-3-acetic acid (AUX/IAA), Waxy (Wx), Acetolactate Synthase (ALS), OsERF922, OsSWEET13, OsSWEET14, TaMLO, GL2, betaine aldehyde dehydrogenase (BADH2), Matrilineal (MTL), Frigida, Grain Weight 2 (GW2), Gn1a, DEP1, GS3, SlMLO1, SlJAZ2, CsLOB1, EDR1, Self-Pruning 5G (SPSG), Slagamous-Like 6 (SlAGL6), thermosensitive genic male-sterile 5 gene (TMS5), OsMATL, ARGOS8, eukaryotic translation initiation factor 4E (eIF4E), granule-bound starch synthase (GBSS) or vacuolar invertase (VInv). In some embodiments, the targeted DNA binding protein is selected from the group of: a recombinase, a helicase, a zinc finger protein, a transcription activator-like effectors (TALE), and a topoisomerase. In some embodiments, a targeted DNA binding protein has no intrinsic nucleic acid cleavage activity.

In one aspect, this disclosure provides a method of increasing allelic diversity in a targeted region of a nucleic acid molecule within a genome of a prokaryote, comprising providing to the prokaryote: (a) a targeted DNA binding protein or a nucleic acid encoding the targeted DNA binding protein where the targeted DNA binding protein binds to a targeted region of the nucleic acid molecule; and (b) at least one mutagen; where the allelic diversity of the targeted region of the nucleic acid is increased. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light. In some embodiments, the prokaryote is selected from an Escherichia coli, a Bacillus subtilis, a Bacillus thuringiensis, a Bacillus coagulans, a Thermus aquaticus, and a Pseudomonas chlororaphis. In some embodiments, allelic diversity is increased in a nucleic acid encoding an insecticidal toxin. In some embodiments, the targeted DNA binding protein is selected from the group of: a recombinase, a helicase, a zinc finger protein, a transcription activator-like effectors (TALE), and a topoisomerase. In some embodiments, a targeted DNA binding protein has no intrinsic nucleic acid cleavage activity.

In one aspect, this disclosure provides a method of providing a plant with an improved agronomic characteristic, comprising: (a) providing to a first plant: (i) a targeted DNA binding protein or a nucleic acid encoding the targeted DNA binding protein where the targeted DNA binding protein binds to a targeted region of a nucleic acid molecule in a genome of the plant; and (ii) at least one mutagen; where at least one modification is induced in the targeted region of the nucleic acid molecule; (b) generating at least one progeny plant from the first plant; and (c) selecting at least one progeny plant comprising the at least one modification and the improved agronomic characteristic. In some embodiments, the mutagen is a chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the mutagen is a physical mutagen. In some embodiments, the physical mutagen is ionizing radiation. In some embodiments, the physical mutagen is X-ray. In some embodiments, the physical mutagen is visible light. In some embodiments, the physical mutagen is heat. In some embodiments, the physical mutagen is UV light. In some embodiments, the plant is selected from corn, cotton, soybean, canola, wheat, barley, rice, tomato, onion, pepper, blueberry, raspberry, blackberry, strawberry, watermelon, cucurbit, brassica, spinach, eggplant, potato, sweet potato, sugar cane, oat, millet, rye, flax, alfalfa, and beet. In some embodiments, the improved agronomic characteristic is selected from the group consisting of: disease resistance, abiotic stress tolerance, insect resistance, oil content, height, drought resistance, maturity, lodging resistance, kernel weight, and yield. In some embodiments, the targeted DNA binding protein is selected from the group of: a recombinase, a helicase, a zinc finger protein, a transcription activator-like effectors (TALE), and a topoisomerase. In some embodiments, a targeted DNA binding protein has no intrinsic nucleic acid cleavage activity.

In one aspect, this disclosure provides a kit for inducing a targeted modification in a target nucleic acid molecule, comprising: (a) a targeted DNA binding protein, or a nucleic acid encoding the targeted DNA binding protein; and (b) at least one chemical mutagen. In some embodiments, the chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitro so-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide. In some embodiments, the kit further comprises one or more of: a DNA-targeting nucleic acid, a reagent for reconstitution and/or dilution. In some embodiments, the kit further comprises a reagent selected from the group consisting of: a buffer for introducing the targeted DNA binding protein into cells, a wash buffer, a control reagent, a control expression vector or RNA polynucleotide, a reagent for in vitro production of the targeted DNA binding protein from DNA, a reagent for in vitro production of the targeted DNA binding protein from DNA, Agrobacterium, and combinations thereof. In some embodiments, the targeted DNA binding protein is selected from the group of: a recombinase, a helicase, a zinc finger protein, a transcription activator-like effectors (TALE), and a topoisomerase. In some embodiments, a targeted DNA binding protein has no intrinsic nucleic acid cleavage activity.

### DETAILED DESCRIPTION

The present disclosure relates to compositions and methods utilizing a catalytically inactive guided-nuclease (e.g., without being limiting, a catalytically inactive CRISPR-associated protein, such as dead Cas9 or dead Cpf1, paired with a guide nucleic acid targeting a nucleic acid sequence) in combination with a mutagen to enrich for mutations within a targeted sequence.

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of the embodiments described in this disclosure includes, unless otherwise indicated, utilize conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N. F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

As used herein, terms in the singular and the singular forms “a,” “an,” and “the,” for example, include plural referents unless the context clearly dictates otherwise.

Any composition, nucleic acid molecule, polypeptide, cell, plant, etc. provided herein is specifically envisioned for use with any method provided herein.

In one aspect, this disclosure provides a method of inducing a targeted modification in a target nucleic acid, comprising contacting the target nucleic acid with: (a) a catalytically inactive guided-nuclease; and (b) at least one mutagen, where at least one modification is induced in the target nucleic acid. In a further aspect, a method provided herein further comprises (c) at least one guide nucleic acid, where the at least one guide nucleic acid forms a complex with the catalytically inactive guided-nuclease, and where the at least one guide nucleic acid hybridizes with the target nucleic acid molecule.

In one aspect, this disclosure provides a method of inducing a targeted modification in a target nucleic acid, comprising contacting the target nucleic acid with: (a) a targeted DNA binding protein; and (b) at least one mutagen, where at least one modification is induced in the target nucleic acid. In a further aspect, a method provided herein further comprises (c) at least one guide nucleic acid, where the at least one guide nucleic acid forms a complex with the catalytically inactive guided-nuclease, and where the at least one guide nucleic acid hybridizes with the target nucleic acid molecule.

In one aspect, this disclosure provides a method of inducing a targeted modification in a target nucleic acid, comprising contacting the target nucleic acid with (a) a catalytically inactive guided-nuclease; (b) at least one guide nucleic acid, where the at least one guide nucleic acid forms a complex with the catalytically inactive guided-nuclease, and where the at least one guide nucleic acid hybridizes with the target nucleic acid; and (c) at least one mutagen, where the target nucleic acid comprises a protospacer adjacent motif (PAM) site, and where at least one modification is induced in the target nucleic acid within 100 nucleotides of the PAM site.

In one aspect, this disclosure provides a method of increasing the activity of a mutagen at a targeted location in the genome, comprising contacting the genome with: (a) a catalytically inactive guided-nuclease; and (b) at least one mutagen, where the rate of mutation is increased at the targeted location as compared to a non-targeted location in the genome. In a further aspect, a method provided herein further comprises (c) at least one guide nucleic acid, where the at least one guide nucleic acid forms a complex with the catalytically inactive guided-nuclease, and where the at least one guide nucleic acid hybridizes within or adjacent to the targeted location.

In one aspect, this disclosure provides a method of increasing the activity of a mutagen at a targeted location in the genome, comprising contacting the genome with: (a) a targeted DNA binding protein, wherein the targeted DNA binding protein binds DNA within or adjacent to the targeted location; and (b) at least one mutagen, where the rate of mutation is increased at the targeted location as compared to a non-targeted location in the genome.

In one aspect, this disclosure provides a kit for inducing a targeted modification in a target nucleic acid, comprising: (a) a catalytically inactive guided-nuclease, or a nucleic acid encoding the catalytically inactive guided-nuclease; and (b) at least one chemical mutagen. In a further aspect, a kit provided herein further comprises (c) at least one guide nucleic acid or a nucleic acid encoding the at least one guide nucleic acid, where the at least one guide nucleic acid forms a complex with the catalytically inactive guided-nuclease, and where the at least one guide nucleic acid hybridizes with the target nucleic acid molecule.

In one aspect, this disclosure provides a kit for inducing a targeted modification in a target nucleic acid, comprising: (a) a targeted DNA binding protein, or a nucleic acid encoding the targeted DNA binding protein; and (b) at least one chemical mutagen.

In one aspect, this disclosure provides a method of increasing allelic diversity in a target region of a genome of a plant, comprising providing to the plant: (a) a catalytically inactive guided-nuclease or a nucleic acid encoding the catalytically inactive guided-nuclease; (b) at least one guide nucleic acid or a nucleic acid encoding the guide nucleic acid, where the at least one guide nucleic acid forms a complex with the catalytically inactive guided-nuclease, and where the at least one guide nucleic acid hybridizes with the target nucleic acid molecule; and (c) at least one mutagen, where the target region is adjacent a protospacer adjacent motif (PAM) site, and where allelic diversity in the target region of the plant genome is increased. In some embodiments, the PAM is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides 3′ of the targeted region of the nucleic acid. In some embodiments, the PAM is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides 5′ of the targeted region of the nucleic acid.

As used herein, a “nuclease” refers to an enzyme that is capable of cleaving at least one phosphodiester bond between two nucleotides. As used herein, “nuclease activity” refers to the cleavage of a nucleic acid molecule. Measurement of nuclease activity can be accomplished using any appropriate method standard in the art. In an aspect, a nuclease is an endonuclease. In another aspect, a nuclease is an exonuclease. In another aspect, a nuclease is a deoxyribonuclease. In another aspect, a nuclease is a ribonuclease. In an aspect, a nuclease cleaves single-stranded deoxyribonucleic acid (DNA). In another aspect, a nuclease cleaves double-stranded DNA. In an aspect, a nuclease cleaves single-stranded ribonucleic acid (RNA). In a further aspect, a nuclease cleaves double-stranded RNA. In an aspect, a nuclease cleaves a single strand of double-stranded DNA. In an aspect, a nuclease cleaves a single strand of double-stranded RNA. In an aspect, a nuclease cleaves both strands of double-stranded DNA. In an aspect, a nuclease cleaves both strands of double-stranded RNA. In an aspect, a nuclease forms a complex with a guide nucleic acid.

Any nuclease known in the art that specifically binds to a target nucleic acid sequence or can be guided to a target nucleic acid sequence is specifically envisioned. In some embodiments to catalytic activity of the nuclease may be diminished or eliminated. In an aspect, a nuclease is catalogued by the Nomenclature Committee of the International Union of Biochemistry and Molecular Biology (see The Enzyme Database at www(dot)enzyme-database(dot)org; and McDonald et al., Nucleic Acids Res., 37:D593-D597 (2009)) under EC 3.1 and its sub-groups.

Without being limited by any scientific theory, nucleases that are bound to double-stranded DNA (dsDNA), either directly or indirectly, partially unwind, or open, the conformation of the DNA in the vicinity of the nuclease binding site. When a nuclease is bound to dsDNA, and the dsDNA is partially unwound, the dsDNA is more accessible to mutagens.

As used herein, a “catalytically inactive nuclease” refers to a nuclease comprising a domain that retains the ability to bind its target nucleic acid but has a diminished, or eliminated, ability to cleave a nucleic acid molecule, as compared to a control nuclease. In an aspect, a catalytically inactive nuclease is derived from a “control” or “wild type” nuclease. As used herein, a “control” nuclease refers to a naturally-occurring nuclease that can be used as a point of comparison for a catalytically inactive nuclease. In some embodiments, the catalytically inactive nuclease is a catalytically inactive Cas9. In some embodiments, the catalytically inactive Cas9 produces a nick in the targeting strand. In some embodiments, the catalytically inactive Cas9 comprises an Alanine substitution of key residues in the RuvC domain (D10A). In some embodiments, the catalytically inactive Cas9 produces a nick in the nontargeting strand. In some embodiments, the catalytically inactive Cas9 comprises a H840A mutation of the HNH domain. In some embodiments, the catalytically inactive Cas9, known as dead Cas9 (dCas9), lacks all nuclease activity. In some embodiments, the catalytically inactive Cas9 comprises both D10A/H840A mutations. In some embodiments, the catalytically inactive nuclease is a catalytically inactive Cpf1 (also known as Cas12a). In some embodiments, the catalytically inactive Cpf1 produces a nick in the targeting strand. In some embodiments, the catalytically inactive Cpf1 produces a nick in the nontargeting strand. In some embodiments, the catalytically inactive Cpf1, known as dead Cpf1 (dCpf1), lacks all DNase activity. In some embodiments, the catalytically inactive Cpf1 comprises a R1226A mutation in the Nuc domain. In some embodiments, the catalytically inactive Cpf1 comprises an E993A mutation in the RuvC domain, wherein the DNase activities against both strands of target DNA is eliminated. In some embodiments, the catalytically inactive Cpf1 is a dead Cpf1 endonuclease from Acidaminococcus sp. BV3L6 (dAsCpf1).

In some embodiments, the catalytically inactive nuclease is a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, or a catalytically inactive Csf4.

In addition to nucleases, any “targeted DNA binding protein” that unwinds DNA to expose and make the DNA base accessible for modification can be used with the provided methods and kits. Non-limiting examples of “targeted DNA binding proteins” include recombinases, helicases, zinc fingers, transcription activator-like effectors (TALEs), and topoisomerases. In some embodiments, a targeted DNA binding protein may have no intrinsic nucleic acid cleavage activity.

In an aspect, a “targeted DNA binding protein” can be used in place of a “catalytically inactive guided-nuclease” in methods and kits provided herein.

As used herein, “diminished” ability to cleave a nucleic acid molecule refers to a reduction in nuclease activity of at least 50% as compared to a control nuclease. As used herein, “eliminated” ability to cleave a nucleic acid molecule refers to nuclease activity being undetectable using methods standard in the art.

In an aspect, a catalytically inactive nuclease has less than 50% of the nuclease activity of a control nuclease. In another aspect a catalytically inactive nuclease has less than 25% of the nuclease activity of a control nuclease. In another aspect a catalytically inactive nuclease has less than 20% of the nuclease activity of a control nuclease. In another aspect a catalytically inactive nuclease has less than 15% of the nuclease activity of a control nuclease. In another aspect a catalytically inactive nuclease has less than 10% of the nuclease activity of a control nuclease. In another aspect a catalytically inactive nuclease has less than 7.5% of the nuclease activity of a control nuclease. In another aspect a catalytically inactive nuclease has less than 5% of the nuclease activity of a control nuclease. In another aspect a catalytically inactive nuclease has less than 4% of the nuclease activity of a control nuclease. In another aspect a catalytically inactive nuclease has less than 3% of the nuclease activity of a control nuclease. In another aspect a catalytically inactive nuclease has less than 2% of the nuclease activity of a control nuclease. In another aspect a catalytically inactive nuclease has less than 1% of the nuclease activity of a control nuclease. In another aspect a catalytically inactive nuclease has less than 0.5% of the nuclease activity of a control nuclease. In another aspect a catalytically inactive nuclease has less than 0.1% of the nuclease activity of a control nuclease. In another aspect a catalytically inactive nuclease has no detectable nuclease activity.

As a non-limiting example, a dead Cpf1 nuclease can comprise one or more amino acid mutations as compared to a control Cpf1 nuclease. In an aspect, a nuclease provided herein is a dead nuclease.

In an aspect, a catalytically inactive nuclease comprises an amino acid sequence at least 99.9% identical or similar to an amino acid sequence of a control nuclease. In an aspect, a catalytically inactive nuclease comprises an amino acid sequence at least 99.5% identical or similar to an amino acid sequence of a control nuclease. In an aspect, a catalytically inactive nuclease comprises an amino acid sequence at least 99% identical or similar to an amino acid sequence of a control nuclease. In an aspect, a catalytically inactive nuclease comprises an amino acid sequence at least 98% identical or similar to an amino acid sequence of a control nuclease. In an aspect, a catalytically inactive nuclease comprises an amino acid sequence at least 97% identical or similar to an amino acid sequence of a control nuclease. In an aspect, a catalytically inactive nuclease comprises an amino acid sequence at least 96% identical or similar to an amino acid sequence of a control nuclease. In an aspect, a catalytically inactive nuclease comprises an amino acid sequence at least 95% identical or similar to an amino acid sequence of a control nuclease. In an aspect, a catalytically inactive nuclease comprises an amino acid sequence at least 94% identical or similar to an amino acid sequence of a control nuclease. In an aspect, a catalytically inactive nuclease comprises an amino acid sequence at least 93% identical or similar to an amino acid sequence of a control nuclease. In an aspect, a catalytically inactive nuclease comprises an amino acid sequence at least 92% identical or similar to an amino acid sequence of a control nuclease. In an aspect, a catalytically inactive nuclease comprises an amino acid sequence at least 91% identical or similar to an amino acid sequence of a control nuclease. In an aspect, a catalytically inactive nuclease comprises an amino acid sequence at least 90% identical or similar to an amino acid sequence of a control nuclease.

In an aspect, the amino acid sequence of a catalytically inactive nuclease comprises at least one amino acid mutation as compared to the amino acid sequence of a control nuclease. In an aspect, the amino acid sequence of a catalytically inactive nuclease comprises at least two amino acid mutations as compared to the amino acid sequence of a control nuclease. In an aspect, the amino acid sequence of a catalytically inactive nuclease comprises at least three amino acid mutations as compared to the amino acid sequence of a control nuclease. In an aspect, the amino acid sequence of a catalytically inactive nuclease comprises at least four amino acid mutations as compared to the amino acid sequence of a control nuclease. In an aspect, the amino acid sequence of a catalytically inactive nuclease comprises at least five amino acid mutations as compared to the amino acid sequence of a control nuclease.

In an aspect, a catalytically inactive nuclease is unable to cleave a single-stranded nucleic acid or a double-stranded nucleic acid. In another aspect, a catalytically inactive nuclease is unable to cleave DNA. In a further aspect, a catalytically inactive nuclease is unable to cleave RNA. In an aspect, a catalytically inactive nuclease interacts with DNA. In an aspect, a catalytically inactive nuclease interacts with RNA. In an aspect, a catalytically inactive nuclease binds or hybridizes with DNA. In another aspect, a catalytically inactive nuclease binds or hybridizes with RNA. In an aspect, a catalytically inactive nuclease binds a target nucleic acid molecule. In an aspect, a catalytically inactive nuclease binds RNA. In an aspect, a catalytically inactive nuclease binds DNA. In an aspect, a catalytically inactive nuclease forms a complex with a guide nucleic acid. In an aspect, a catalytically inactive nuclease forms a complex with a guide RNA. See Example 2 below for more information regarding catalytically inactive guided-nucleases.

As used herein, a “guided-nuclease” refers to a nuclease whose catalytic domain is guided to a specific target nucleic acid sequence for binding and cleavage. In an aspect, a guided nuclease used herein is a catalytically inactive guided-nuclease that can still bind its target nucleic acid, but has diminished or eliminated activity to cleave the target nucleic acid molecule. In an aspect, a guided nuclease used herein is a catalytically inactive guided-nuclease that can still bind its target nucleic acid, but only cleaves one strand of a double-stranded DNA molecule. In an aspect, a catalytically inactive guided-nuclease binds a single-stranded nucleic acid. In another aspect, a catalytically inactive guided-nuclease binds a double-stranded nucleic acid. In a further aspect, a catalytically inactive guided-nuclease binds an RNA molecule. In another aspect, a catalytically inactive guided-nuclease binds a DNA molecule. In an aspect, a catalytically inactive guided-nuclease binds a single-stranded RNA molecule. In an aspect, a catalytically inactive guided-nuclease binds a single-stranded DNA molecule. In an aspect, a catalytically inactive guided-nuclease binds a double-stranded RNA molecule. In an aspect, a catalytically inactive guided-nuclease binds a double-stranded DNA molecule.

In an aspect, a guided-nuclease further comprises a nucleic acid-binding domain that specifically recognizes and binds a target nucleic acid sequence. In one aspect, the nucleic acid-binding domain is a DNA-binding domain. In another aspect, the nucleic acid-biding domain is an RNA-binding domain.

In an aspect, a catalytically inactive guided-nuclease further comprises a DNA-binding domain. In another aspect, a catalytically inactive guided-nuclease further comprises an RNA-binding domain. In a further aspect, a catalytically inactive guided-nuclease forms a complex with a guide nucleic acid. In another aspect, a catalytically inactive guided-nuclease forms a complex with a guide DNA. In an aspect, a catalytically inactive guided-nuclease forms a complex with a guide RNA.

In one aspect, the catalytically inactive guided-nuclease is guided to a target nucleic acid molecule via a direct interaction between the catalytically inactive guided-nuclease and the target nucleic acid molecule. A direct interaction between a catalytically inactive guided-nuclease and a target nucleic acid molecule refers to amino acids from the catalytically inactive guided-nuclease forming covalent or non-covalent interactions with the target nucleic acid molecule. Without being limited by any theory, in this type of direct interaction a DNA-binding domain or motif of the catalytically inactive guided-nuclease can recognize and bind, hybridize, or interact with a specific nucleic acid sequence within the target nucleic acid molecule.

In another aspect, the catalytically inactive guided-nuclease is guided to a specific sequence in target nucleic acid molecule via a guide nucleic acid. Without being limited by any theory, in this type of interaction a guide nucleic acid can form a complex with the catalytically inactive guided-nuclease, and the guide nucleic acid can bind, hybridize, or interact with the target nucleic acid molecule in a sequence specific manner.

In an aspect, a catalytically inactive guided-nuclease is a catalytically inactive CRISPR (clustered regularly interspaced short palindromic repeats) associated protein. As used herein, a “CRISPR associated protein (CRISPR-Cas)” refers to any nuclease derived from the CRISPR family of nucleases found in bacteria and archaea species. In some embodiments, the CRISPR-Cas is a Class 1 CRISPR-Cas. In some embodiments, the CRISPR-Cas is a Class 1 CRISPR-Cas selected from the group consisting of Type I, Type IA, Type IB, Type IC, Type ID, Type IE, Type IF, Type IU, Type III, Type IIIA, Type IIIB, Type IIIC, Type IIID, Type IV, Type IVA, Type IVB. In some embodiments, the CRISPR-Cas is a Class 2 CRISPR-Cas. In some embodiments, the CRISPR-Cas is a Class 2 CRISPR-Cas selected from the group consisting of Type II, Type IIA, Type IIB, Type ITC, Type V, Type VI. In an aspect, a catalytically inactive CRISPR associated protein is selected from the group consisting of a catalytically inactive Cas9, a catalytically inactive Cpf1 (also known as Cas12a), a catalytically inactive CasX, a catalytically inactive CasY, a catalytically inactive C2c2. In an aspect, a catalytically inactive CRISPR associated protein is a catalytically inactive Cas9. In an aspect, a catalytically inactive CRISPR associated protein is a dead Cpf1. In an aspect, a catalytically inactive CRISPR associated protein is a catalytically inactive CasX. In an aspect, a catalytically inactive CRISPR associated protein is a catalytically inactive CasY. In an aspect, a catalytically inactive CRISPR associated protein is a catalytically inactive C2c2. In an aspect, a catalytically inactive CRISPR associated protein is a catalytically inactive Streptococcus pyogenes Cas9 (SpCas9). In another aspect, a catalytically inactive CRISPR associated protein is a catalytically inactive Lachnospiraceae bacterium Cpf1 (LbCpf1). In another aspect, a catalytically inactive CRISPR associated protein comprises an amino acid sequence of SEQ ID NO: 22 dSpCas9 PTN. In another aspect, a catalytically inactive CRISPR associated protein comprises an amino acid sequence of SEQ ID NO: 24 dLbCpf1 PTN.

In an aspect, a catalytically inactive CRISPR associated protein is selected from the group consisting of a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, and a catalytically inactive Csf4.

In an aspect, a catalytically inactive CRISPR associated protein binds a guide nucleic acid. In another aspect, a catalytically inactive CRISPR associated protein binds a guide RNA. In an aspect, a catalytically inactive CRISPR associated protein forms a complex with a guide nucleic acid. In another aspect, a catalytically inactive CRISPR associated protein forms a complex with a guide RNA. In some embodiments, the guide nucleic acid comprises a targeting sequence that, together with a catalytically inactive CRISPR associated protein, provides for sequence-specific targeting of a nucleic acid sequence.

In some embodiments, the guide nucleic acid comprises: a first segment comprising a nucleotide sequence that is complementary to a sequence in a target nucleic acid and a second segment that interacts with a catalytically inactive CRISPR associated protein. In some embodiments, the first segment of a guide comprising a nucleotide sequence that is complementary to a sequence in a target nucleic acid corresponds to a CRISPR RNA (crRNA or crRNA repeat). In some embodiments, the second segment of a guide comprising a nucleic acid sequence that interacts with a catalytically inactive CRISPR associated protein corresponds to a trans-acting CRISPR RNA (tracrRNA). In some embodiments, the guide nucleic acid comprises two separate nucleic acid molecules (a polynucleotide that is complementary to a sequence in a target nucleic acid and a polynucleotide that interacts with a catalytically inactive CRISPR associated protein) that hybridize with one another and is referred to herein as a “double-guide” or a “two-molecule guide”. In some embodiments, the double-guide may comprise DNA, RNA or a combination of DNA and RNA. In other embodiments, the guide nucleic acid is a single polynucleotide and is referred to herein as a “single-molecule guide” or a “single-guide”. In some embodiments, the single-guide may comprise DNA, RNA or a combination of DNA and RNA. The term “guide nucleic acid” is inclusive, referring both to double-molecule guides and to single-molecule guides.

In an aspect, a guide nucleic acid provided herein can be expressed from a recombinant vector in vivo. In an aspect, a guide nucleic acid provided herein can be expressed from a recombinant vector in vitro. In an aspect, a guide nucleic acid provided herein can be expressed from a recombinant vector ex vivo. In an aspect, a guide nucleic acid provided herein can be expressed from a nucleic acid molecule in vivo. In an aspect, a guide nucleic acid provided herein can be expressed from a nucleic acid molecule in vitro. In an aspect, a guide nucleic acid provided herein can be expressed from a nucleic acid molecule ex vivo. In another aspect, a guide nucleic acid provided herein can be synthetically synthesized.

In an aspect, a catalytically inactive CRISPR associated protein comprises a catalytically inactive Cas9 derived from a bacteria genus selected from the group consisting of Streptococcus, Haloferax, Anabaena, Mycobacterium, Aeropyvrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacteriunm, Streptomyces, Aquifex, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In another aspect, a catalytically inactive CRISPR associated protein comprises a catalytically inactive Cpf1 derived from a bacteria genus selected from the group consisting of Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Acidaminococcus, Peregrinibacteria, Butyrivibrio, Parcubacteria, Smithella, Candidatus, Moraxella, and Leptospira.

In another aspect, a catalytically inactive guided-nuclease is selected from the group consisting of a catalytically inactive meganuclease, a catalytically inactive zinc-finger nuclease, and a catalytically inactive transcription activator-like effector nuclease (TALEN). In an aspect, a catalytically inactive guided-nuclease is a catalytically inactive meganuclease. In an aspect, a catalytically inactive guided-nuclease is a catalytically inactive zinc-finger nuclease. In another aspect, a catalytically inactive guided-nuclease is a catalytically inactive TALEN.

In an aspect, a catalytically inactive meganuclease binds a target nucleic acid molecule. In an aspect, a catalytically inactive zinc-finger nuclease binds a target nucleic acid molecule. In an aspect, a catalytically inactive TALEN binds a target nucleic acid molecule. In an aspect, a zinc-finger protein binds a target nucleic acid molecule. In an aspect, a TALE protein binds a target nucleic acid molecule.

In an aspect, a catalytically inactive guided-nuclease provided herein can be expressed from a recombinant vector in vivo. In an aspect, a catalytically inactive guided-nuclease provided herein can be expressed from a recombinant vector in vitro. In an aspect, a catalytically inactive guided-nuclease provided herein can be expressed from a recombinant vector ex vivo. In an aspect, a catalytically inactive guided-nuclease provided herein can be expressed from a nucleic acid molecule in vivo. In an aspect, a catalytically inactive guided-nuclease provided herein can be expressed from a nucleic acid molecule in vitro. In an aspect, a catalytically inactive guided-nuclease provided herein can be expressed from a nucleic acid molecule ex vivo. In another aspect, a catalytically inactive guided-nuclease provided herein can be synthetically synthesized.

As used herein, “codon optimization” refers to a process of modifying a nucleic acid sequence for enhanced expression in a host cell of interest by replacing at least one codon (e.g., at least 1, 2, 3, 4, 5, 10, 15, 20, 25, 50, or more codons) of a sequence with codons that are more frequently or most frequently used in the genes of the host cell while maintaining the original amino acid sequence (e.g., introducing silent mutations). Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www(dot)kazusa(dot)or(dot)jp/codon and these tables can be adapted in a number of ways. See Nakamura et al., 2000, Nucl. Acids Res. 28:292. Computer algorithms for codon optimizing a particular sequence for expression in a particular host cell are also available, such as Gene Forge (Aptagen; Jacobus, Pa.), are also available. As to codon usage in plants, including algae, reference is made to Campbell and Gowri, 1990, Plant Physiol., 92: 1-11; and Murray et al., 1989, Nucleic Acids Res., 17:477-98.

In an aspect, a nucleic acid encoding a catalytically inactive guided-nuclease is codon optimized for a prokaryotic cell. In another aspect, a nucleic acid encoding a catalytically inactive guided-nuclease is codon optimized for an Escherichia coli cell. In another aspect, a nucleic acid encoding a catalytically inactive guided-nuclease is codon optimized for a eukaryotic cell. In another aspect, a nucleic acid encoding a catalytically inactive guided-nuclease is codon optimized for an animal cell. In another aspect, a nucleic acid encoding a catalytically inactive guided-nuclease is codon optimized for a human cell. In another aspect, a nucleic acid encoding a catalytically inactive guided-nuclease is codon optimized for a mouse cell. In another aspect, a nucleic acid encoding a catalytically inactive guided-nuclease is codon optimized for a Caenorhabditis elegans cell. In another aspect, a nucleic acid encoding a catalytically inactive guided-nuclease is codon optimized for a Drosophila melanogaster cell. In another aspect, a nucleic acid encoding a catalytically inactive guided-nuclease is codon optimized for a pig cell. In another aspect, a nucleic acid encoding a catalytically inactive guided-nuclease is codon optimized for a mammal cell. In another aspect, a nucleic acid encoding a catalytically inactive guided-nuclease is codon optimized for an insect cell. In another aspect, a nucleic acid encoding a catalytically inactive guided-nuclease is codon optimized for a cephalopod cell. In another aspect, a nucleic acid encoding a catalytically inactive guided-nuclease is codon optimized for an arthropod cell. In another aspect, a nucleic acid encoding a catalytically inactive guided-nuclease is codon optimized for a plant cell. In another aspect, a nucleic acid encoding a catalytically inactive guided-nuclease is codon optimized for a corn cell. In another aspect, a nucleic acid encoding a catalytically inactive guided-nuclease is codon optimized for a rice cell. In another aspect, a nucleic acid encoding a catalytically inactive guided-nuclease is codon optimized for a wheat cell. In another aspect, a nucleic acid encoding a catalytically inactive guided-nuclease is codon optimized for a soybean cell. In another aspect, a nucleic acid encoding a catalytically inactive guided-nuclease is codon optimized for a cotton cell. In another aspect, a nucleic acid encoding a catalytically inactive guided-nuclease is codon optimized for an alfalfa cell. In another aspect, a nucleic acid encoding a catalytically inactive guided-nuclease is codon optimized for a barley cell. In another aspect, a nucleic acid encoding a catalytically inactive guided-nuclease is codon optimized for a sorghum cell. In another aspect, a nucleic acid encoding a catalytically inactive guided-nuclease is codon optimized for a sugarcane cell. In another aspect, a nucleic acid encoding a catalytically inactive guided-nuclease is codon optimized for a canola cell. In another aspect, a nucleic acid encoding a catalytically inactive guided-nuclease is codon optimized for a tomato cell. In another aspect, a nucleic acid encoding a catalytically inactive guided-nuclease is codon optimized for an Arabidopsis cell. In another aspect, a nucleic acid encoding a catalytically inactive guided-nuclease is codon optimized for a cucumber cell. In another aspect, a nucleic acid encoding a catalytically inactive guided-nuclease is codon optimized for a potato cell. In another aspect, a nucleic acid encoding a catalytically inactive guided-nuclease is codon optimized for an algae cell. In another aspect, a nucleic acid encoding a catalytically inactive guided-nuclease is codon optimized for a grass cell. In another aspect, a nucleic acid encoding a catalytically inactive guided-nuclease is codon optimized for a monocotyledonous plant cell. In another aspect, a nucleic acid encoding a catalytically inactive guided-nuclease is codon optimized for a dicotyledonous plant cell. In another aspect, a nucleic acid encoding a catalytically inactive guided-nuclease is codon optimized for a gymnosperm plant cell.

In some embodiments, a nucleic acid encoding a catalytically inactive dead guided-nuclease may be optimized for delivery by biolistics. As used herein, a “cys-free LbCpf1” refers to an LbCpf1 protein variant wherein the 9 cysteines present in the native LbCpf1 sequence (WO2016205711-1150) are all mutated. In an aspect the cys-free LbCpf1 comprises the following 9 amino acid substitutions when compared to a wt LbCpf1 protein sequence: C10L, C175L, C565S, C632L, C805A, C912V, C965S, C1090P, C1116L. Cysteine residues in a protein are able to form disulfide bridges providing a strong reversible attachment between cysteines. To control and direct the attachment of Cpf1 in a targeted manner the native cysteines must be removed to control the formation of these bridges. Removal of the cysteines from the protein backbone would enable targeted insertion of new cysteine residues to control the placement of these reversible connections by a disulfide linkage. This could be between protein domains or to a particle such as a gold particle for biolistic delivery. A tag comprising several residues of cysteine could be added to the cys-free LbCpf1 that would allow it to specifically attach to metal beads (specifically gold) in a uniform way.

It can be desirable to direct a catalytically inactive guided-nuclease to the nucleus of a cell. In such instances, one or more nuclear localization signals can be used to direct the localization of the catalytically inactive guided-nuclease. As used herein, a “nuclear localization signal” refers to an amino acid sequence that “tags” a protein (e.g., a catalytically inactive guided-nuclease) for import into the nucleus of a cell. In an aspect, a nucleic acid molecule provided herein encodes a nuclear localization signal. In another aspect, a nucleic acid molecule provided herein encodes two or more nuclear localization signals. In an aspect, a catalytically inactive guided-nuclease provided herein comprises a nuclear localization signal. In an aspect, a nuclear localization signal is positioned on the N-terminal end of a catalytically inactive guided-nuclease. In a further aspect, a nuclear localization signal is positioned on the C-terminal end of a catalytically inactive guided-nuclease. In yet another aspect, a nuclear localization signal is positioned on both the N-terminal end and the C-terminal end of a catalytically inactive guided-nuclease.

While not being limited by any particular scientific theory, a CRISPR associated protein forms a complex with a guide nucleic acid, which hybridizes with a complementary sequence in a target nucleic acid molecule, thereby guiding the CRISPR associated protein to the target nucleic acid molecule. In class 2 CRISPR-Cas systems, CRISPR arrays, including spacers, are transcribed during encounters with recognized invasive DNA and are processed into small interfering CRISPR RNAs (crRNAs). The crRNA comprises a repeat sequence and a spacer sequence which is complementary to a specific protospacer sequence in an invading pathogen. The spacer sequence can be designed to be complementary to target sequences in a eukaryotic genome. CRISPR associated proteins associate with their respective crRNAs in their active forms.

When the CRISPR associated protein and a guide RNA form a complex, the whole system is called a “ribonucleoprotein.” The guide RNA guides the ribonucleoprotein to a complementary target sequence, where the CRISPR associated protein cleaves either one or two strands of DNA. Depending on the protein, cleavage can occur within a certain number of nucleotides (e.g., between 18-23 nucleotides for Cpf1) from a PAM site. PAM sites are only required for Type I and Type II CRISPR associated proteins; Type III CRISPR associated proteins do not require a PAM site for proper targeting or cleavage.

In an aspect, any method or kit provided herein that requires (a) a catalytically inactive guided-nuclease and (b) a guide nucleic acid, is specifically envisioned to provide (a) and (b) as a ribonucleoprotein.

In an aspect, a method or kit provided herein comprises a ribonucleoprotein. In an aspect, a ribonucleoprotein comprises a catalytically inactive guided-nuclease and a guide nucleic acid. In another aspect, a ribonucleoprotein comprises a catalytically inactive CRISPR associated protein and a guide nucleic acid. In another aspect, a ribonucleoprotein comprises a catalytically inactive Cas9 protein and a guide nucleic acid. In another aspect, a ribonucleoprotein comprises a catalytically inactive Cpf1 protein and a guide nucleic acid. In another aspect, a ribonucleoprotein comprises a catalytically inactive CasX protein and a guide nucleic acid.

In an aspect, a ribonucleoprotein comprises a catalytically inactive guided-nuclease and a guide RNA. In another aspect, a ribonucleoprotein comprises a catalytically inactive CRISPR associated protein and a guide RNA. In another aspect, a ribonucleoprotein comprises a catalytically inactive Cas9 protein and a guide RNA. In another aspect, a ribonucleoprotein comprises a catalytically inactive Cpf1 protein and a guide RNA. In another aspect, a ribonucleoprotein comprises a catalytically inactive CasX protein and a guide RNA.

In an aspect, a ribonucleoprotein is generated in vivo. In another aspect, a ribonucleoprotein is generated in vitro. In a further aspect, a ribonucleoprotein is generated ex vivo.

In an aspect, a ribonucleoprotein is delivered to a cell. In another aspect, a ribonucleoprotein is introduced to a cell. In another aspect, a ribonucleoprotein is introduced to a plant cell by bombardment.

### Modifications

As used herein, a “modification” refers to an insertion, deletion, substitution, duplication, or inversion of one or more amino acids or nucleotides as compared to a reference amino acid sequence or to a reference nucleotide sequence. A “targeted modification” refers to a modification occurring within a targeted region of a nucleic acid molecule.

In an aspect, a modification comprises a substitution. In another aspect, a modification comprises an insertion. In another aspect, a modification comprises a deletion. In another aspect, a modification is selected from the group consisting of a substitution, an insertion, and a deletion. In an aspect, a modification occurs in vivo. In another aspect, a modification occurs in vitro. In a further aspect, a modification occurs ex vivo. In an aspect, a modification occurs in genomic DNA. In an aspect, a modification occurs in chromosomal DNA.

As used herein, the term “INDEL” refers to insertion and/or deletion of one or more nucleotides in genomic DNA. INDELs include insertions and/or deletions of a single nucleotide up to insertions and/or deletions less than 1 kb in length. Where an INDEL is not divisible by 3, an INDEL can change the reading frame, resulting in a completely different translation from the original sequence due to the triplet nature of gene expression by codons.

In an aspect, a modification comprises the insertion of at least one nucleotide. In another aspect, a modification comprises the insertion of at least two nucleotides. In another aspect, a modification comprises the insertion of at least five nucleotides. In another aspect, a modification comprises the insertion of at least 10 nucleotides. In another aspect, a modification comprises the insertion of at least 25 nucleotides. In another aspect, a modification comprises the insertion of at least 50 nucleotides. In another aspect, a modification comprises the insertion of at least 75 nucleotides. In another aspect, a modification comprises the insertion of at least 100 nucleotides. In another aspect, a modification comprises the insertion of at least 250 nucleotides. In another aspect, a modification comprises the insertion of at least 500 nucleotides. In another aspect, a modification comprises the insertion of at least 1000 nucleotides.

In an aspect, a modification comprises the deletion of at least one nucleotide. In another aspect, a modification comprises the deletion of at least two nucleotides. In another aspect, a modification comprises the deletion of at least five nucleotides. In another aspect, a modification comprises the deletion of at least 10 nucleotides. In another aspect, a modification comprises the deletion of at least 25 nucleotides. In another aspect, a modification comprises the deletion of at least 50 nucleotides. In another aspect, a modification comprises the deletion of at least 75 nucleotides. In another aspect, a modification comprises the deletion of at least 100 nucleotides. In another aspect, a modification comprises the deletion of at least 250 nucleotides. In another aspect, a modification comprises the deletion of at least 500 nucleotides. In another aspect, a modification comprises the deletion of at least 1000 nucleotides.

In an aspect, a modification comprises the substitution of at least one nucleotide. In another aspect, a modification comprises the substitution of at least two nucleotides. In another aspect, a modification comprises the substitution of at least five nucleotides. In another aspect, a modification comprises the substitution of at least 10 nucleotides. In another aspect, a modification comprises the substitution of at least 25 nucleotides. In another aspect, a modification comprises the substitution of at least 50 nucleotides. In another aspect, a modification comprises the substitution of at least 75 nucleotides. In another aspect, a modification comprises the substitution of at least 100 nucleotides. In another aspect, a modification comprises the substitution of at least 250 nucleotides. In another aspect, a modification comprises the substitution of at least 500 nucleotides. In another aspect, a modification comprises the substitution of at least 1000 nucleotides.

In an aspect, a modification comprises the inversion of at least two nucleotides. In another aspect, a modification comprises the inversion of at least five nucleotides. In another aspect, a modification comprises the inversion of at least 10 nucleotides. In another aspect, a modification comprises the inversion of at least 25 nucleotides. In another aspect, a modification comprises the inversion of at least 50 nucleotides. In another aspect, a modification comprises the inversion of at least 75 nucleotides. In another aspect, a modification comprises the inversion of at least 100 nucleotides. In another aspect, a modification comprises the inversion of at least 250 nucleotides. In another aspect, a modification comprises the inversion of at least 500 nucleotides. In another aspect, a modification comprises the inversion of at least 1000 nucleotides.

In several embodiments, the target nucleic acid comprises a PAM sequence. As used herein, a “PAM site” or “PAM sequence” refers to a short DNA sequence (usually 2-6 base pairs in length) that is adjacent to the DNA region targeted for cleavage by a CRISPR associate protein/guide nucleic acid system, such as CRISPR-Cas9 or CRISPR-Cpf1. Some CRISPR associated proteins (e.g., Type I and Type II) require a PAM site in order to bind a target nucleic acid.

In one aspect, a modification in a targeted region of a nucleic acid molecule is induced within 1000 nucleotides of a PAM. In another aspect, a modification in a targeted region of a nucleic acid molecule is induced within 750 nucleotides of a PAM. In another aspect, a modification in a targeted region of a nucleic acid molecule is induced within 500 nucleotides of a PAM. In another aspect, a modification in a targeted region of a nucleic acid molecule is induced within 250 nucleotides of a PAM. In another aspect, a modification in a targeted region of a nucleic acid molecule is induced within 200 nucleotides of a PAM. In another aspect, a modification in a targeted region of a nucleic acid molecule is induced within 100 nucleotides of a PAM. In another aspect, a modification in a targeted region of a nucleic acid molecule is induced within 75 nucleotides of a PAM. In another aspect, a modification in a targeted region of a nucleic acid molecule is induced within 50 nucleotides of a PAM. In another aspect, a modification in a targeted region of a nucleic acid molecule is induced within 40 nucleotides of a PAM. In another aspect, a modification in a targeted region of a nucleic acid molecule is induced within 35 nucleotides of a PAM. In another aspect, a modification in a targeted region of a nucleic acid molecule is induced within 30 nucleotides of a PAM. In another aspect, a modification in a targeted region of a nucleic acid molecule is induced within 25 nucleotides of a PAM. In another aspect, a modification in a targeted region of a nucleic acid molecule is induced within 20 nucleotides of a PAM. In another aspect, a modification in a targeted region of a nucleic acid molecule is induced within 19 nucleotides of a PAM. In another aspect, a modification in a targeted region of a nucleic acid molecule is induced within 18 nucleotides of a PAM. In another aspect, a modification in a targeted region of a nucleic acid molecule is induced within 17 nucleotides of a PAM. In another aspect, a modification in a targeted region of a nucleic acid molecule is induced within 16 nucleotides of a PAM. In another aspect, a modification in a targeted region of a nucleic acid molecule is induced within 15 nucleotides of a PAM. In another aspect, a modification in a targeted region of a nucleic acid molecule is induced within 14 nucleotides of a PAM. In another aspect, a modification in a targeted region of a nucleic acid molecule is induced within 13 nucleotides of a PAM. In another aspect, a modification in a targeted region of a nucleic acid molecule is induced within 12 nucleotides of a PAM. In another aspect, a modification in a targeted region of a nucleic acid molecule is induced within 11 nucleotides of a PAM. In another aspect, a modification in a targeted region of a nucleic acid molecule is induced within 10 nucleotides of a PAM. In another aspect, a modification in a targeted region of a nucleic acid molecule is induced within 9 nucleotides of a PAM. In another aspect, a modification in a targeted region of a nucleic acid molecule is induced within 8 nucleotides of a PAM. In another aspect, a modification in a targeted region of a nucleic acid molecule is induced within 7 nucleotides of a PAM. In another aspect, a modification in a targeted region of a nucleic acid molecule is induced within 6 nucleotides of a PAM. In another aspect, a modification in a targeted region of a nucleic acid molecule is induced within 5 nucleotides of a PAM. In another aspect, a modification in a targeted region of a nucleic acid molecule is induced within 4 nucleotides of a PAM. In another aspect, a modification in a targeted region of a nucleic acid molecule is induced within 3 nucleotides of a PAM. In another aspect, a modification in a targeted region of a nucleic acid molecule is induced within 2 nucleotides of a PAM. In another aspect, a modification in a targeted region of a nucleic acid molecule is induced within 1 nucleotides of a PAM. In another aspect, a modification in a targeted region of a nucleic acid molecule is induced between 1 nucleotides and 750 nucleotides from a PAM. In another aspect, a modification in a targeted region of a nucleic acid molecule is induced between 1 nucleotides and 250 nucleotides from a PAM. In another aspect, a modification in a targeted region of a nucleic acid molecule is induced between 1 nucleotides and 100 nucleotides from a PAM. In another aspect, a modification in a targeted region of a nucleic acid molecule is induced between 1 nucleotides and 50 nucleotides from a PAM. In another aspect, a modification in a targeted region of a nucleic acid molecule is induced between 1 nucleotides and 25 nucleotides from a PAM. In another aspect, a modification in a targeted region of a nucleic acid molecule is induced between 10 nucleotides and 50 nucleotides from a PAM.

In an aspect, a target nucleic acid molecule comprises at least one PAM. In another aspect, a target nucleic acid molecule comprises at least two PAMs. In another aspect, a target nucleic acid molecule comprises at least five PAMs. In a further aspect, a target nucleic acid molecule comprises between one PAM and 50 PAMs. Without being limited by any theory, some guided-nuclease, such as CRISPR associated proteins, require the presence of a specific PAM in a target nucleic acid molecule in order for a complex comprising a guide nucleic acid and a CRISPR associated protein to bind to the targeted region of the nucleic acid molecule. In one aspect, a PAM comprises a nucleotide sequence of 5′-NGG-3′. In another aspect, a PAM comprises a nucleotide sequence of 5′-NGA-3′. In another aspect, a PAM comprises a nucleotide sequence of 5′-TTTN-3′. In another aspect, a PAM comprises a nucleotide sequence of 5′-TTTV-3′. In another aspect, a PAM comprises a nucleotide sequence of 5′-YG-3′. In another aspect, a PAM comprises a nucleotide sequence of 5′-YTN-3′. In another aspect, a PAM comprises a nucleotide sequence of 5′-TTCN-3′. In another aspect, a PAM comprises a nucleotide sequence of 5′-NGAN-3′. In another aspect, a PAM comprises a nucleotide sequence of 5′-NGNG-3′. In another aspect, a PAM comprises a nucleotide sequence of 5′-NGAG-3′. In another aspect, a PAM comprises a nucleotide sequence of 5′-NGCG-3′. In another aspect, a PAM comprises a nucleotide sequence of 5′-TYCV-3′. In another aspect, a PAM comprises a nucleotide sequence of 5′-NGRRT-3′. In another aspect, a PAM comprises a nucleotide sequence of 5′-NGRRN-3′. In another aspect, a PAM comprises a nucleotide sequence of 5′-NNNNGATT-3′. In another aspect, a PAM comprises a nucleotide sequence of 5′-NNNNRYAC-3′. In another aspect, a PAM comprises a nucleotide sequence of 5′-NNAGAAW-3′. In another aspect, a PAM comprises a nucleotide sequence of 5′-NAAAAC-3′. As is known in the art, in regards to nucleotides, “A” refers to adenine; “T” refers to thymine; “C” refers to cytosine; “G” refers to guanine; “N” refers to any nucleotide; “R” refers to adenine or guanine; “Y” refers to cytosine or thymine; “V” refers to adenine, guanine, or cytosine; and “W” refers to adenine or thymine.

The screening and selection of modified nucleic acid molecules, or cells comprising modified nucleic acid molecules, can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

### Mutagens

In an aspect, a method or kit provided herein comprises at least one mutagen. As used herein, a “mutagen” refers to any agent that is capable of generating a modification, or mutation, to a nucleic acid sequence. In one aspect, a mutagen increases the frequency of mutations above the natural background level. In one aspect, a mutagen is a chemical mutagen. In one aspect, a mutagen is a physical mutagen. Physical mutagens exert their mutagenic effects by causing breaks in the DNA backbone. In another aspect, a mutagen is ionizing radiation. In another aspect, a mutagen is ultraviolet radiation. In another aspect, a mutagen is alpha-particle radiation. In another aspect, a mutagen is beta-particle radiation. In another aspect, a mutagen is Gamma-ray radiation. In another aspect, a mutagen is electromagnetic radiation. In another aspect, a mutagen is neutron radiation. In another aspect, a mutagen is a reactive oxygen species. In another aspect, a mutagen is a deaminating agent. In another aspect, a mutagen is an alkylating agent. In another aspect, a mutagen is an aromatic amine. In another aspect, a mutagen is and intercalcating agent, such as ethidium bromide or proflavin. In another aspect, a mutagen is X-rays. In another aspect, a mutagen is UVA radiation. In another aspect, a mutagen is UVB radiation. In another aspect, a mutagen is visible light. In another aspect, a mutagen is selected from the group consisting of a chemical mutagen and ionizing radiation.

In an aspect, a chemical mutagen is selected from the group consisting of ethyl methanesulfonate (EMS), methyl methanesulfonate, diethylsulphonate, dimethyl sulfate, dimethyl sulfoxide, diethylnitrosamine, N-nitroso-N-methylurea, N-methyl-N-nitrosourea, N-nitro so-N-diethyl urea, N-ethyl-N-nitrosourea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, psoralen, benzene, Datura extract, bromodeoxyuridine, and beryllium oxide.

In another aspect, a chemical mutagen is provided at a concentration of at least 0.000001%. In another aspect, a chemical mutagen is provided at a concentration of at least 0.000005%. In another aspect, a chemical mutagen is provided at a concentration of at least 0.00001%. In another aspect, a chemical mutagen is provided at a concentration of at least 0.00005%. In another aspect, a chemical mutagen is provided at a concentration of at least 0.0001%. In another aspect, a chemical mutagen is provided at a concentration of at least 0.0005%. In another aspect, a chemical mutagen is provided at a concentration of at least 0.001%. In another aspect, a chemical mutagen is provided at a concentration of at least 0.005%. In another aspect, a chemical mutagen is provided at a concentration of at least 0.01%. In another aspect, a chemical mutagen is provided at a concentration of at least 0.05%. In another aspect, a chemical mutagen is provided at a concentration of at least 0.1%. In another aspect, a chemical mutagen is provided at a concentration of at least 0.5%. In another aspect, a chemical mutagen is provided at a concentration of at least 1%. In another aspect, a chemical mutagen is provided at a concentration of at least 5%. In another aspect, a chemical mutagen is provided at a concentration of at least 10%. In another aspect, a chemical mutagen is provided at a concentration of between 0.0001% and 1%. In another aspect, a chemical mutagen is provided at a concentration of between 0.001% and 1%. In another aspect, a chemical mutagen is provided at a concentration of between 0.01% and 1%. In another aspect, a chemical mutagen is provided at a concentration of between 0.1% and 1%. In another aspect, a chemical mutagen is provided at a concentration of between 0.01% and 5%. In another aspect, a chemical mutagen is provided at a concentration of between 0.01% and 10%. In another aspect, a chemical mutagen is provided at a concentration of between 1% and 5%. In another aspect, a chemical mutagen is provided at a concentration of between 1% and 10%.

In an aspect, a chemical mutagen is provided in a gaseous form. In another aspect, a chemical mutagen is provided in a liquid form. In another aspect, a chemical mutagen is provided in a solid form. In another aspect, a chemical mutagen is provided in a crystallized form. In another aspect, a chemical mutagen is provided in a powdered form.

Some chemical mutagens are known to be capable of causing modification of individual nucleotides in a nucleic acid sequence. Specific types of substitutions are referred to as transversions (e.g., a point mutation in a nucleic acid sequence where a purine is changed to a pyrimidine; or where a pyrimidine is changed to a purine) or transitions (e.g., a point mutation in a nucleic acid sequence where a purine is changed to a different purine; or where a pyrimidine is changed to a different pyrimidine). Non-limiting examples of purines include adenine and guanine. Non-limiting examples of pyrimidines include cytosine, thymine, and uracil.

In an aspect, a substitution comprises a substitution of a guanine for an adenine. In another aspect, a substitution comprises a substitution of a guanine for a cytosine. In another aspect, a substitution comprises a substitution of a guanine for a thymine. In another aspect, a substitution comprises a substitution of an adenine for a guanine. In another aspect, a substitution comprises a substitution of an adenine for a cytosine. In another aspect, a substitution comprises a substitution of an adenine for a thymine. In another aspect, a substitution comprises a substitution of a cytosine for a guanine. In another aspect, a substitution comprises a substitution of a cytosine for an adenine. In another aspect, a substitution comprises a substitution of a cytosine for a thymine. In another aspect, a substitution comprises a substitution of a thymine for a guanine. In another aspect, a substitution comprises a substitution of a thymine for an adenine. In another aspect, a substitution comprises a substitution of a thymine for a cytosine.

In an aspect, ionizing radiation is selected from the group consisting of X-ray radiation, gamma ray radiation, alpha particle radiation, and ultraviolet (UV) radiation.

In an aspect, a chemical mutagen is provided to a cell concurrently with a ribonucleoprotein. In another aspect, a chemical mutagen is provided to a cell before a ribonucleoprotein is provided to a cell. In a further aspect, a chemical mutagen is provided to a cell after a ribonucleoprotein is provided to a cell.

In an aspect, a chemical mutagen is provided to a cell concurrently with a catalytically inactive guided-nuclease. In another aspect, a chemical mutagen is provided to a cell before a catalytically inactive guided-nuclease is provided to a cell. In a further aspect, a chemical mutagen is provided to a cell after a catalytically inactive guided-nuclease is provided to a cell.

In an aspect, a chemical mutagen is provided to a cell concurrently with a guide nucleic acid. In another aspect, a chemical mutagen is provided to a cell before a guide nucleic acid is provided to a cell. In a further aspect, a chemical mutagen is provided to a cell after a guide nucleic acid is provided to a cell.

In an aspect, a chemical mutagen is provided to a cell expressing a catalytically inactive guided-nuclease. In another aspect, a chemical mutagen is provided to a cell expressing a guide nucleic acid.

In an aspect, a physical mutagen is provided to a cell concurrently with a ribonucleoprotein. In another aspect, a physical mutagen is provided to a cell before a ribonucleoprotein is provided to a cell. In a further aspect, a physical mutagen is provided to a cell after a ribonucleoprotein is provided to a cell.

In an aspect, a physical mutagen is provided to a cell concurrently with a catalytically inactive guided-nuclease. In another aspect, a physical mutagen is provided to a cell before a catalytically inactive guided-nuclease is provided to a cell. In a further aspect, a physical mutagen is provided to a cell after a catalytically inactive guided-nuclease is provided to a cell.

In an aspect, a physical mutagen is provided to a cell concurrently with a guide nucleic acid. In another aspect, a physical mutagen is provided to a cell before a guide nucleic acid is provided to a cell. In a further aspect, a physical mutagen is provided to a cell after a guide nucleic acid is provided to a cell.

### Allelic Diversity

The methods and kits provided in this disclosure can be used to increase the allelic diversity of a targeted locus within a genome. As used herein, “allelic diversity” refers to the number of alleles of a given locus in a genome. Increasing allelic diversity results from generating alleles via modification at a target locus.

In an aspect, this disclosure provides a method of increasing allelic diversity in a targeted region of a nucleic acid molecule within a genome of a plant, comprising providing to the plant: (a) a catalytically inactive guided-nuclease or a nucleic acid encoding the catalytically guided-nuclease; (b) at least one guide nucleic acid or a nucleic acid encoding the at least one guide nucleic acid, where the at least one guide nucleic acid forms a complex with the catalytically inactive guided-nuclease, and where the at least one guide nucleic acid hybridizes with the nucleic acid molecule; and (c) at least one mutagen; where the nucleic acid comprises a protospacer adjacent motif (PAM), and where allelic diversity of the target nucleic acid is increased.

In an aspect, increased allelic diversity comprises the generation of at least one modified allele of a target nucleic acid as compared to an unmodified wild-type target nucleic acid. In an aspect, increased allelic diversity comprises the generation of at least two modified alleles of a target nucleic acid as compared to an unmodified wild-type target nucleic acid. In an aspect, increased allelic diversity comprises the generation of at least three modified alleles of a target nucleic acid as compared to an unmodified wild-type target nucleic acid. In an aspect, increased allelic diversity comprises the generation of at least four modified alleles of a target nucleic acid as compared to an unmodified wild-type target nucleic acid. In an aspect, increased allelic diversity comprises the generation of at least five modified alleles of a target nucleic acid as compared to an unmodified wild-type target nucleic acid. In an aspect, increased allelic diversity comprises the generation of at least 10 modified alleles of a target nucleic acid as compared to an unmodified wild-type target nucleic acid. In an aspect, increased allelic diversity comprises the generation of at least 15 modified alleles of a target nucleic acid as compared to an unmodified wild-type target nucleic acid. In an aspect, increased allelic diversity comprises the generation of at least 20 modified alleles of a target nucleic acid as compared to an unmodified wild-type target nucleic acid. In an aspect, increased allelic diversity comprises the generation of at least 30 modified alleles of a target nucleic acid as compared to an unmodified wild-type target nucleic acid. In an aspect, increased allelic diversity comprises the generation of at least 50 modified alleles of a target nucleic acid as compared to an unmodified wild-type target nucleic acid.

Allelic series can be useful for identifying modifications that produce optimal plant traits. As used herein, an “allelic series” refers to two or more different modifications within a targeted locus, where the two or more different modifications cause two or more different phenotypes.

In an aspect, a method or kit provided herein produces an allelic series in an R₀ generation. In an aspect, a method or kit provided herein produces an allelic series in an R₁ generation. In an aspect, an allelic series comprises at least one recessive modification. In another aspect, an allelic series comprises at least one dominant modification. As used herein, a “recessive modification” refers to a modification that only produces a phenotype when present in a genome in a homozygous state. In contrast, a “dominant modification” refers to a modification that produces a phenotype when present in a genome in a heterozygous state.

In an aspect, a method or kit provided herein comprises the generation of an average of at least 0.001 modifications in a target nucleic acid per 100 R₀ plants produced. In an aspect, a method or kit provided herein comprises the generation of an average of at least 0.0025 modifications in a target nucleic acid per 100 R₀ plants produced. In an aspect, a method or kit provided herein comprises the generation of an average of at least 0.005 modifications in a target nucleic acid per 100 R₀ plants produced. In an aspect, a method or kit provided herein comprises the generation of an average of at least 0.0075 modifications in a target nucleic acid per 100 R₀ plants produced. In an aspect, a method or kit provided herein comprises the generation of an average of at least 0.01 modifications in a target nucleic acid per 100 R₀ plants produced. In an aspect, a method or kit provided herein comprises the generation of an average of at least 0.025 modifications in a target nucleic acid per 100 R₀ plants produced. In an aspect, a method or kit provided herein comprises the generation of an average of at least 0.05 modifications in a target nucleic acid per 100 R₀ plants produced. In an aspect, a method or kit provided herein comprises the generation of an average of at least 0.075 modifications in a target nucleic acid per 100 R₀ plants produced. In an aspect, a method or kit provided herein comprises the generation of an average of at least 0.1 modifications in a target nucleic acid per 100 R₀ plants produced. In an aspect, a method or kit provided herein comprises the generation of an average of at least 0.25 modifications in a target nucleic acid per 100 R₀ plants produced. In an aspect, a method or kit provided herein comprises the generation of an average of at least 0.5 modifications in a target nucleic acid per 100 R₀ plants produced. In an aspect, a method or kit provided herein comprises the generation of an average of at least 0.75 modifications in a target nucleic acid per 100 R₀ plants produced. In an aspect, a method or kit provided herein comprises the generation of an average of at least 1 modifications in a target nucleic acid per 100 R₀ plants produced. In an aspect, a method or kit provided herein comprises the generation of an average of at least 2.5 modifications in a target nucleic acid per 100 R₀ plants produced. In an aspect, a method or kit provided herein comprises the generation of an average of at least 5 modifications in a target nucleic acid per 100 R₀ plants produced. In an aspect, a method or kit provided herein comprises the generation of an average of at least 7.5 modifications in a target nucleic acid per 100 R₀ plants produced. In an aspect, a method or kit provided herein comprises the generation of an average of at least 10 modifications in a target nucleic acid per 100 R₀ plants produced.

### Mutation Rate

In an aspect, a method or kit provided herein provides an increased mutation rate as compared to the background mutation rate at the targeted region. As used herein, “mutation rate” refers to the frequency with which a wild-type sequence is modified in a control cell. Typically, mutation rates are expressed as the number of mutations per cellular division. The calculation of mutation rates is well known in the art and can vary for different parts of a genome.

In humans, for example, the background mutation rate has been estimated to be approximately 1.1×10⁻⁸ per site per cellular generation. Maize has been estimated to have an average background mutation rate of approximately 7.7×10⁻⁵ per site per generation. See, for example, Drake et al., “Rates of Spontaneous Mutation,” Genetics, 148:1667-1686 (1998).

In an aspect, this disclosure provides a method of increasing the mutation rate of a targeted region of a nucleic acid molecule, comprising contacting the nucleic acid molecule with: (a) a catalytically inactive guided-nuclease; (b) at least one guide nucleic acid, where the at least one guide nucleic acid forms a complex with the catalytically inactive guided-nuclease, and where the at least one guide nucleic acid hybridizes with the nucleic acid molecule; and (c) at least one mutagen; where the nucleic acid comprises a protospacer adjacent motif (PAM) site adjacent to the targeted region of the nucleic acid molecule, and where the mutation rate in the targeted region of the nucleic acid molecule is increased as compared to an untargeted region of the nucleic acid molecule.

In an aspect, a method or kit provided herein increases the mutation rate of a target nucleic acid by at least 1×10⁻⁹ per site per cellular generation as compared to the background mutation rate. In an aspect, a method or kit provided herein increases the mutation rate of a target nucleic acid by at least 5×10⁻⁹ per site per cellular generation as compared to the background mutation rate. In an aspect, a method or kit provided herein increases the mutation rate of a target nucleic acid by at least 25×10⁻⁹ per site per cellular generation as compared to the background mutation rate.

In an aspect, a method or kit provided herein increases the mutation rate of a target nucleic acid by at least 1×10⁻⁸ per site per cellular generation as compared to the background mutation rate. In an aspect, a method or kit provided herein increases the mutation rate of a target nucleic acid by at least 5×10⁻⁸ per site per cellular generation as compared to the background mutation rate. In an aspect, a method or kit provided herein increases the mutation rate of a target nucleic acid by at least 25×10⁻⁸ per site per cellular generation as compared to the background mutation rate.

In an aspect, a method or kit provided herein increases the mutation rate of a target nucleic acid by at least 1×10⁻⁷ per site per cellular generation as compared to the background mutation rate. In an aspect, a method or kit provided herein increases the mutation rate of a target nucleic acid by at least 5×10⁻⁷ per site per cellular generation as compared to the background mutation rate. In an aspect, a method or kit provided herein increases the mutation rate of a target nucleic acid by at least 25×10⁻⁷ per site per cellular generation as compared to the background mutation rate.

In an aspect, a method or kit provided herein increases the mutation rate of a target nucleic acid by at least 1×10⁻⁶ per site per cellular generation as compared to the background mutation rate. In an aspect, a method or kit provided herein increases the mutation rate of a target nucleic acid by at least 5×10⁻⁶ per site per cellular generation as compared to the background mutation rate. In an aspect, a method or kit provided herein increases the mutation rate of a target nucleic acid by at least 25×10⁻⁶ per site per cellular generation as compared to the background mutation rate.

In an aspect, a method or kit provided herein increases the mutation rate of a target nucleic acid by at least 1×10⁻⁵ per site per cellular generation as compared to the background mutation rate. In an aspect, a method or kit provided herein increases the mutation rate of a target nucleic acid by at least 5×10⁻⁵ per site per cellular generation as compared to the background mutation rate. In an aspect, a method or kit provided herein increases the mutation rate of a target nucleic acid by at least 25×10⁻⁵ per site per cellular generation as compared to the background mutation rate.

In an aspect, a method or kit provided herein increases the mutation rate of a target nucleic acid by at least 1×10⁻⁴ per site per cellular generation as compared to the background mutation rate. In an aspect, a method or kit provided herein increases the mutation rate of a target nucleic acid by at least 5×10⁻⁴ per site per cellular generation as compared to the background mutation rate. In an aspect, a method or kit provided herein increases the mutation rate of a target nucleic acid by at least 25×10⁻⁴ per site per cellular generation as compared to the background mutation rate.

In an aspect, a method or kit provided herein increases the mutation rate of a target nucleic acid by at least 1×10⁻³ per site per cellular generation as compared to the background mutation rate. In an aspect, a method or kit provided herein increases the mutation rate of a target nucleic acid by at least 5×10⁻³ per site per cellular generation as compared to the background mutation rate. In an aspect, a method or kit provided herein increases the mutation rate of a target nucleic acid by at least 25×10⁻³ per site per cellular generation as compared to the background mutation rate.

In an aspect, a method or kit provided herein increases the mutation rate of a target nucleic acid by at least 1×10⁻⁹ per site per cellular generation as compared to an untargeted nucleic acid. In an aspect, a method or kit provided herein increases the mutation rate of a target nucleic acid by at least 5×10⁻⁹ per site per cellular generation as compared to an untargeted nucleic acid. In an aspect, a method or kit provided herein increases the mutation rate of a target nucleic acid by at least 25×10⁻⁹ per site per cellular generation as compared to an untargeted nucleic acid.

In an aspect, a method or kit provided herein increases the mutation rate of a target nucleic acid by at least 1×10⁻⁸ per site per cellular generation as compared to an untargeted nucleic acid. In an aspect, a method or kit provided herein increases the mutation rate of a target nucleic acid by at least 5×10⁻⁸ per site per cellular generation as compared to an untargeted nucleic acid. In an aspect, a method or kit provided herein increases the mutation rate of a target nucleic acid by at least 25×10⁻⁸ per site per cellular generation as compared to an untargeted nucleic acid.

In an aspect, a method or kit provided herein increases the mutation rate of a target nucleic acid by at least 1×10⁻⁷ per site per cellular generation as compared to an untargeted nucleic acid. In an aspect, a method or kit provided herein increases the mutation rate of a target nucleic acid by at least 5×10⁻⁷ per site per cellular generation as compared to an untargeted nucleic acid. In an aspect, a method or kit provided herein increases the mutation rate of a target nucleic acid by at least 25×10⁻⁷ per site per cellular generation as compared to an untargeted nucleic acid.

In an aspect, a method or kit provided herein increases the mutation rate of a target nucleic acid by at least 1×10⁻⁶ per site per cellular generation as compared to an untargeted nucleic acid. In an aspect, a method or kit provided herein increases the mutation rate of a target nucleic acid by at least 5×10⁻⁶ per site per cellular generation as compared to an untargeted nucleic acid. In an aspect, a method or kit provided herein increases the mutation rate of a target nucleic acid by at least 25×10⁻⁶ per site per cellular generation as compared to an untargeted nucleic acid.

In an aspect, a method or kit provided herein increases the mutation rate of a target nucleic acid by at least 1×10⁻⁵ per site per cellular generation as compared to an untargeted nucleic acid. In an aspect, a method or kit provided herein increases the mutation rate of a target nucleic acid by at least 5×10⁻⁵ per site per cellular generation as compared to an untargeted nucleic acid. In an aspect, a method or kit provided herein increases the mutation rate of a target nucleic acid by at least 25×10⁻⁵ per site per cellular generation as compared to an untargeted nucleic acid.

In an aspect, a method or kit provided herein increases the mutation rate of a target nucleic acid by at least 1×10⁻⁴ per site per cellular generation as compared to an untargeted nucleic acid. In an aspect, a method or kit provided herein increases the mutation rate of a target nucleic acid by at least 5×10⁻⁴ per site per cellular generation as compared to an untargeted nucleic acid. In an aspect, a method or kit provided herein increases the mutation rate of a target nucleic acid by at least 25×10⁻⁴ per site per cellular generation as compared to an untargeted nucleic acid.

In an aspect, a method or kit provided herein increases the mutation rate of a target nucleic acid by at least 1×10⁻³ per site per cellular generation as compared to an untargeted nucleic acid. In an aspect, a method or kit provided herein increases the mutation rate of a target nucleic acid by at least 5×10⁻³ per site per cellular generation as compared to an untargeted nucleic acid. In an aspect, a method or kit provided herein increases the mutation rate of a target nucleic acid by at least 25×10⁻³ per site per cellular generation as compared to an untargeted nucleic acid.

By increasing the mutation rate for a targeted region, it is envisioned that fewer plants will need to be screened in order to identify a modification in a targeted region.

In an aspect, a method or kit provided herein requires fewer plants be screened to identify a modification in a targeted region as compared to using a chemical mutagen alone in the absence of a catalytically inactive guided-nuclease. In an aspect, a method or kit provided herein requires fewer plants be screened to identify a modification in a targeted region as compared to using EMS alone in the absence of a catalytically inactive guided-nuclease.

In an aspect, a method or kit provided herein produces an average of at least one modification in a targeted region for every one plant produced in the R₀ generation. In an aspect, a method or kit provided herein produces an average of at least one modification in a targeted region for every two plants produced in the R₀ generation. In an aspect, a method or kit provided herein produces an average of at least one modification in a targeted region for every three plants produced in the R₀ generation. In an aspect, a method or kit provided herein produces an average of at least one modification in a targeted region for every four plants produced in the R₀ generation. In an aspect, a method or kit provided herein produces an average of at least one modification in a targeted region for every five plants produced in the R₀ generation. In an aspect, a method or kit provided herein produces an average of at least one modification in a targeted region for every six plants produced in the R₀ generation. In an aspect, a method or kit provided herein produces an average of at least one modification in a targeted region for every seven plants produced in the R₀ generation. In an aspect, a method or kit provided herein produces an average of at least one modification in a targeted region for every eight plants produced in the R₀ generation. In an aspect, a method or kit provided herein produces an average of at least one modification in a targeted region for every nine plants produced in the R₀ generation. In an aspect, a method or kit provided herein produces an average of at least one modification in a targeted region for every ten plants produced in the R₀ generation. In an aspect, a method or kit provided herein produces an average of at least one modification in a targeted region for every 15 plants produced in the R₀ generation. In an aspect, a method or kit provided herein produces an average of at least one modification in a targeted region for every 20 plants produced in the R₀ generation. In an aspect, a method or kit provided herein produces an average of at least one modification in a targeted region for every 25 plants produced in the R₀ generation. In an aspect, a method or kit provided herein produces an average of at least one modification in a targeted region for every 30 plants produced in the R₀ generation. In an aspect, a method or kit provided herein produces an average of at least one modification in a targeted region for every 35 plants produced in the R₀ generation. In an aspect, a method or kit provided herein produces an average of at least one modification in a targeted region for every 40 plants produced in the R₀ generation. In an aspect, a method or kit provided herein produces an average of at least one modification in a targeted region for every 50 plants produced in the R₀ generation. In an aspect, a method or kit provided herein produces an average of at least one modification in a targeted region for every 75 plants produced in the R₀ generation. In an aspect, a method or kit provided herein produces an average of at least one modification in a targeted region for every 100 plants produced in the R₀ generation. In an aspect, a method or kit provided herein produces an average of at least one modification in a targeted region for every 150 plants produced in the R₀ generation. In an aspect, a method or kit provided herein produces an average of at least one modification in a targeted region for every 200 plants produced in the R₀ generation. In an aspect, a method or kit provided herein produces an average of at least one modification in a targeted region for every 250 plants produced in the R₀ generation. In an aspect, a method or kit provided herein produces an average of at least one modification in a targeted region for every 300 plants produced in the R₀ generation. In an aspect, a method or kit provided herein produces an average of at least one modification in a targeted region for every 400 plants produced in the R₀ generation. In an aspect, a method or kit provided herein produces an average of at least one modification in a targeted region for every 500 plants produced in the R₀ generation.

As used herein, the “R₀ generation” refers to the initial generation created via the methods and kits provided herein. Subsequent generations arising from the R₀ generation would be termed R1, R2, R3, etc.

### Guide Nucleic Acids

In an aspect, a method or kit provided herein comprises at least one guide nucleic acid or a nucleic acid encoding the at least one guide nucleic acid, where the at least one guide nucleic acid forms a complex with the catalytically inactive guided-nuclease, and where the at least one guide nucleic acid hybridizes with the target nucleic acid molecule. As used herein, a “guide nucleic acid” refers to a nucleic acid that forms a complex with a nuclease and then guides the complex to a specific sequence in a target nucleic acid molecule, where the guide nucleic acid and the target nucleic acid molecule share complementary sequences.

In an aspect, a guide nucleic acid comprises DNA. In another aspect, a guide nucleic acid comprises RNA. When a guide nucleic acid comprises RNA, it can be referred to as a “guide RNA.” In another aspect, a guide nucleic acid comprises DNA and RNA. In another aspect, a guide nucleic acid is single-stranded. In another aspect, a guide nucleic acid is double-stranded. In a further aspect, a guide nucleic acid is partially double-stranded.

In another aspect, a guide nucleic acid comprises at least 10 nucleotides. In another aspect, a guide nucleic acid comprises at least 11 nucleotides. In another aspect, a guide nucleic acid comprises at least 12 nucleotides. In another aspect, a guide nucleic acid comprises at least 13 nucleotides. In another aspect, a guide nucleic acid comprises at least 14 nucleotides. In another aspect, a guide nucleic acid comprises at least 15 nucleotides. In another aspect, a guide nucleic acid comprises at least 16 nucleotides. In another aspect, a guide nucleic acid comprises at least 17 nucleotides. In another aspect, a guide nucleic acid comprises at least 18 nucleotides. In another aspect, a guide nucleic acid comprises at least 19 nucleotides. In another aspect, a guide nucleic acid comprises at least 20 nucleotides. In another aspect, a guide nucleic acid comprises at least 21 nucleotides. In another aspect, a guide nucleic acid comprises at least 22 nucleotides. In another aspect, a guide nucleic acid comprises at least 23 nucleotides. In another aspect, a guide nucleic acid comprises at least 24 nucleotides. In another aspect, a guide nucleic acid comprises at least 25 nucleotides. In another aspect, a guide nucleic acid comprises at least 26 nucleotides. In another aspect, a guide nucleic acid comprises at least 27 nucleotides. In another aspect, a guide nucleic acid comprises at least 28 nucleotides. In another aspect, a guide nucleic acid comprises at least 30 nucleotides. In another aspect, a guide nucleic acid comprises at least 35 nucleotides. In another aspect, a guide nucleic acid comprises at least 40 nucleotides. In another aspect, a guide nucleic acid comprises at least 45 nucleotides. In another aspect, a guide nucleic acid comprises at least 50 nucleotides. In another aspect, a guide nucleic acid comprises between 10 nucleotides and 50 nucleotides. In another aspect, a guide nucleic acid comprises between 10 nucleotides and 40 nucleotides. In another aspect, a guide nucleic acid comprises between 10 nucleotides and 30 nucleotides. In another aspect, a guide nucleic acid comprises between 10 nucleotides and 20 nucleotides. In another aspect, a guide nucleic acid comprises between 16 nucleotides and 28 nucleotides. In another aspect, a guide nucleic acid comprises between 16 nucleotides and 25 nucleotides. In another aspect, a guide nucleic acid comprises between 16 nucleotides and 20 nucleotides.

In an aspect, a guide nucleic acid comprises at least 70% sequence complementarity to a target nucleic acid sequence. In an aspect, a guide nucleic acid comprises at least 75% sequence complementarity to a target nucleic acid sequence. In an aspect, a guide nucleic acid comprises at least 80% sequence complementarity to a target nucleic acid sequence. In an aspect, a guide nucleic acid comprises at least 85% sequence complementarity to a target nucleic acid sequence. In an aspect, a guide nucleic acid comprises at least 90% sequence complementarity to a target nucleic acid sequence. In an aspect, a guide nucleic acid comprises at least 91% sequence complementarity to a target nucleic acid sequence. In an aspect, a guide nucleic acid comprises at least 92% sequence complementarity to a target nucleic acid sequence. In an aspect, a guide nucleic acid comprises at least 93% sequence complementarity to a target nucleic acid sequence. In an aspect, a guide nucleic acid comprises at least 94% sequence complementarity to a target nucleic acid sequence. In an aspect, a guide nucleic acid comprises at least 95% sequence complementarity to a target nucleic acid sequence. In an aspect, a guide nucleic acid comprises at least 96% sequence complementarity to a target nucleic acid sequence. In an aspect, a guide nucleic acid comprises at least 97% sequence complementarity to a target nucleic acid sequence. In an aspect, a guide nucleic acid comprises at least 98% sequence complementarity to a target nucleic acid sequence. In an aspect, a guide nucleic acid comprises at least 99% sequence complementarity to a target nucleic acid sequence. In an aspect, a guide nucleic acid comprises 100% sequence complementarity to a target nucleic acid sequence. In another aspect, a guide nucleic acid comprises between 70% and 100% sequence complementarity to a target nucleic acid sequence. In another aspect, a guide nucleic acid comprises between 80% and 100% sequence complementarity to a target nucleic acid sequence. In another aspect, a guide nucleic acid comprises between 90% and 100% sequence complementarity to a target nucleic acid sequence.

Some CRISPR associated protein, such as CasX and Cas9, require another non-coding RNA component, referred to as a trans-activating crRNA (tracrRNA), to have functional activity. Guide nucleic acid molecules provided herein can combine a crRNA and a tracrRNA into one nucleic acid molecule in what is herein referred to as a “single guide RNA” (sgRNA). The gRNA guides the active CasX complex to a target site, where CasX can cleave the target site.

In an aspect, a guide nucleic acid comprises a crRNA. In another aspect, a guide nucleic acid comprises a tracrRNA. In a further aspect, a guide nucleic acid comprises an sgRNA.

In an aspect, a guide nucleic acid provided herein can be expressed from a recombinant vector in vivo. In an aspect, a guide nucleic acid provided herein can be expressed from a recombinant vector in vitro. In an aspect, a guide nucleic acid provided herein can be expressed from a recombinant vector ex vivo. In an aspect, a guide nucleic acid provided herein can be expressed from a nucleic acid molecule in vivo. In an aspect, a guide nucleic acid provided herein can be expressed from a nucleic acid molecule in vitro. In an aspect, a guide nucleic acid provided herein can be expressed from a nucleic acid molecule ex vivo. In another aspect, a guide nucleic acid provided herein can be synthetically synthesized.

### Nucleic Acids and Polypeptides

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise deoxyribonucleotides, ribonucleotides, or combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded.

In one aspect, methods and compositions provided herein comprise a vector. As used herein, the terms “vector” or “plasmid” are used interchangeably and refer to a circular, double-stranded DNA molecule that is physically separate from chromosomal DNA. In one aspect, a plasmid or vector used herein is capable of replication in vivo. In another aspect, a nucleic acid encoding a catalytically inactive guided-nuclease is provided in a vector. In a further aspect, a nucleic acid encoding a guide nucleic acid is provided in a vector. In still yet another aspect, a nucleic acid encoding a catalytically inactive guided-nuclease and a nucleic acid encoding a guide nucleic acid are provided in a single vector.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, N.Y.).

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

The terms “percent sequence complementarity” or “percent complementarity” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

For optimal alignment of sequences to calculate their percent identity, various pair-wise or multiple sequence alignment algorithms and programs are known in the art, such as ClustalW or Basic Local Alignment Search Tool (BLAST®), etc., that can be used to compare the sequence identity or similarity between two or more nucleotide or protein sequences. Although other alignment and comparison methods are known in the art, the alignment and percent identity between two sequences (including the percent identity ranges described above) can be as determined by the ClustalW algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); Larkin M A et al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

As used herein, a first nucleic acid molecule can “hybridize” a second nucleic acid molecule via non-covalent interactions (e.g., Watson-Crick base-pairing) in a sequence-specific, antiparallel manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine pairing with thymine, adenine pairing with uracil, and guanine (G) pairing with cytosine (C) [DNA, RNA]. In addition, it is also known in the art that for hybridization between two RNA molecules (e.g., dsRNA), guanine base pairs with uracil. For example, G/U base-pairing is partially responsible for the degeneracy (i.e., redundancy) of the genetic code in the context of tRNA anti-codon base-pairing with codons in mRNA. In the context of this disclosure, a guanine of a protein-binding segment (dsRNA duplex) of a subject DNA-targeting RNA molecule is considered complementary to an uracil, and vice versa. As such, when a G/U base-pair can be made at a given nucleotide position a protein-binding segment (dsRNA duplex) of a subject DNA-targeting RNA molecule, the position is not considered to be noncomplementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et al.). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST® programs (basic local alignment search tools) and PowerBLAST programs known in the art (see Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Target Nucleic Acids

As used herein, a “target nucleic acid” or “target nucleic acid molecule” or “target nucleic acid sequence” refers to a selected nucleic acid molecule or a selected sequence or region of a nucleic acid molecule in which a modification by a mutagen as described herein is desired.

As used herein, a “target region” or “targeted region” refers to the portion of a target nucleic acid that is modified by a mutagen. In an aspect, a target region is 100% complementary to a guide nucleic acid. In another aspect, a target region is 99% complementary to a guide nucleic acid. In another aspect, a target region is 98% complementary to a guide nucleic acid. In another aspect, a target region is 97% complementary to a guide nucleic acid. In another aspect, a target region is 96% complementary to a guide nucleic acid. In another aspect, a target region is 95% complementary to a guide nucleic acid. In another aspect, a target region is 94% complementary to a guide nucleic acid. In another aspect, a target region is 93% complementary to a guide nucleic acid. In another aspect, a target region is 92% complementary to a guide nucleic acid. In another aspect, a target region is 91% complementary to a guide nucleic acid. In another aspect, a target region is 90% complementary to a guide nucleic acid. In another aspect, a target region is 85% complementary to a guide nucleic acid. In another aspect, a target region is 80% complementary to a guide nucleic acid. In an aspect, a target region is adjacent to a nucleic acid sequence that is 100% complementary to a guide nucleic acid. In another aspect, a target region is adjacent to a nucleic acid sequence that is 99% complementary to a guide nucleic acid. In another aspect, a target region is adjacent to a nucleic acid sequence that is 98% complementary to a guide nucleic acid. In another aspect, a target region is adjacent to a nucleic acid sequence that is 97% complementary to a guide nucleic acid. In another aspect, a target region is adjacent to a nucleic acid sequence that is 96% complementary to a guide nucleic acid. In another aspect, a target region is adjacent to a nucleic acid sequence that is 95% complementary to a guide nucleic acid. In another aspect, a target region is adjacent to a nucleic acid sequence that is 94% complementary to a guide nucleic acid. In another aspect, a target region is adjacent to a nucleic acid sequence that is 93% complementary to a guide nucleic acid. In another aspect, a target region is adjacent to a nucleic acid sequence that is 92% complementary to a guide nucleic acid. In another aspect, a target region is adjacent to a nucleic acid sequence that is 91% complementary to a guide nucleic acid. In another aspect, a target region is adjacent to a nucleic acid sequence that is 90% complementary to a guide nucleic acid. In another aspect, a target region is adjacent to a nucleic acid sequence that is 85% complementary to a guide nucleic acid. In another aspect, a target region is adjacent to a nucleic acid sequence that is 80% complementary to a guide nucleic acid.

In an aspect, a target region comprises at least one PAM site. In an aspect, a target region is adjacent to a nucleic acid sequence that comprises at least one PAM site. In another aspect, a target region is within 5 nucleotides of at least one PAM site. In a further aspect, a target region is within 10 nucleotides of at least one PAM site. In another aspect, a target region is within 15 nucleotides of at least one PAM site. In another aspect, a target region is within 20 nucleotides of at least one PAM site. In another aspect, a target region is within 25 nucleotides of at least one PAM site. In another aspect, a target region is within 30 nucleotides of at least one PAM site.

In an aspect, a target nucleic acid comprises RNA. In another aspect, a target nucleic acid comprises DNA. In an aspect, a target nucleic acid is single-stranded. In another aspect, a target nucleic acid is double-stranded. In an aspect, a target nucleic acid comprises single-stranded RNA. In an aspect, a target nucleic acid comprises single-stranded DNA. In an aspect, a target nucleic acid comprises double-stranded RNA. In an aspect, a target nucleic acid comprises double-stranded DNA. In an aspect, a target nucleic acid comprises genomic DNA. In an aspect, a target nucleic acid is positioned within a nuclear genome. In an aspect, a target nucleic acid comprises chromosomal DNA. In an aspect, a target nucleic acid comprises plasmid DNA. In an aspect, a target nucleic acid is positioned within a plasmid. In an aspect, a target nucleic acid comprises mitochondrial DNA. In an aspect, a target nucleic acid is positioned within a mitochondrial genome. In an aspect, a target nucleic acid comprises plastid DNA. In an aspect, a target nucleic acid is positioned within a plastid genome. In an aspect, a target nucleic acid comprises chloroplast DNA. In an aspect, a target nucleic acid is positioned within a chloroplast genome. In an aspect, a target nucleic acid is positioned within a genome selected from the group consisting of a nuclear genome, a mitochondrial genome, and a plastid genome.

In an aspect, a target nucleic acid encodes a gene. As used herein, a “gene” refers to a polynucleotide that can produce a functional unit (e.g., without being limiting, for example, a protein, or a non-coding RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a non-protein-coding RNA molecule or a precursor thereof. In another aspect, a gene encodes a protein. In some embodiments, the target nucleic acid is selected from the group consisting of: a promoter, an enhancer sequence, a leader sequence, a transcriptional start site, a transcriptional stop site, a polyadenylation site, an exon, an intron, a splice site, a 5′-UTR, a 3′-UTR, a protein coding sequence, a non-protein-coding sequence, a miRNA, a pre-miRNA and a miRNA binding site.

Non-limiting examples of a non-protein-coding RNA molecule include a microRNA (miRNA), a miRNA precursor (pre-miRNA), a small interfering RNA (siRNA), a small RNA (18-26 nt in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single guide RNA (sgRNA).

Non-limiting examples of target nucleic acids in plants include genes encoding Brachytic1, Brachytic2, Brachytic3, Flowering Locus T, Rgh1, Rsp1, Rsp2, Rsp3, 5-Enolpyruvylshikimate-3-Phosphate Synthase (EPSPS), acetohydroxyacid synthase, dihydropteroate synthase, phytoene desaturase (PDS), Protoporphyrin IX oxygenase (PPO), para-aminobenzoate synthase, 1-deoxy-D-xylulose 5-phosphate (DOXP) synthase, dihydropteroate (DHP) synthase, phenylalanine ammonia lyase (PAL), glutathione S-transferase (GST), D1 protein of photosystem II, mono-oxygenase, cytochrome P450, cellulose synthase, beta-tubulin, RUBISCO, translation initiation factor, phytoene desaturase double-stranded DNA adenosine tripolyphosphatase (ddATP), fatty acid desaturase 2 (FAD2), Gibberellin 20 Oxidase (GA20ox), Acetyl-CoA Carboxylase (ACC), Glutamine Synthetase (GS), p-Hydroxyphenylpyruvate Dioxygenase (HPPD), Hydroxymethyldihydropterin Pyrophosphokinase (DHPS), auxin/indole-3-acetic acid (AUX/IAA), Waxy (Wx), Acetolactate Synthase (ALS), OsERF922, OsSWEET13, OsSWEET14, TaMLO, GL2, betaine aldehyde dehydrogenase (BADH2), Matrilineal (MTL), Frigida, Grain Weight 2 (GW2), Gn1a, DEP1, GS3, SlMLO1, SlJAZ2, CsLOB1, EDR1, Self-Pruning 5G (SP5G), Slagamous-Like 6 (SlAGL6), thermosensitive genic male-sterile 5 gene (TMS5), OsMATL, ARGOS8, eukaryotic translation initiation factor 4E (eIF4E), granule-bound starch synthase (GBSS) and vacuolar invertase (VInv).

### Cells

In an aspect, a target nucleic acid is within a cell. In another aspect, a target nucleic acid is within a prokaryotic cell. In an aspect, a prokaryotic cell is a cell from a phylum selected from the group consisting of prokaryotic cell is a cell from a phylum selected from the group consisting of Acidobacteria, Actinobacteria, Aquificae, Armatimonadetes, Bacteroidetes, Caldiserica, Chlamydie, Chlorobi, Chloroflexi, Chrysiogenetes, Coprothermobacterota, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospirae, Planctomycetes, Proteobacteria, Spirochaetes, Synergistetes, Tenericutes, Thermodesulfobacteria, Thermotogae, and Verrucomicrobia. In another aspect, a prokaryotic cell is an Escherichia coli cell. In another aspect, a prokaryotic cell is selected from a genus selected from the group consisting of Escherichia, Agrobacterium, Rhizobium, Sinorhizobium, and Staphylococcus. In another aspect, the prokaryotic cell is selected from a genus selected from the group consisting of Lactobacillus, Bifidobacterium, Streptococcus, Enterococcus, Escherichia, and Bacillus.

In another aspect, a target nucleic acid is within a eukaryotic cell. In a further aspect, a eukaryotic cell is an ex vivo cell. In another aspect, a eukaryotic cell is a yeast cell. In another aspect, a eukaryotic cell is a plant cell. In another aspect, a eukaryotic cell is a plant cell in culture. In another aspect, a eukaryotic cell is an angiosperm plant cell. In another aspect, a eukaryotic cell is a gymnosperm plant cell. In another aspect, a eukaryotic cell is a monocotyledonous plant cell. In another aspect, a eukaryotic cell is a dicotyledonous plant cell. In another aspect, a eukaryotic cell is a corn cell. In another aspect, a eukaryotic cell is a rice cell. In another aspect, a eukaryotic cell is a sorghum cell. In another aspect, a eukaryotic cell is a wheat cell. In another aspect, a eukaryotic cell is a canola cell. In another aspect, a eukaryotic cell is an alfalfa cell. In another aspect, a eukaryotic cell is a soybean cell. In another aspect, a eukaryotic cell is a cotton cell. In another aspect, a eukaryotic cell is a tomato cell. In another aspect, a eukaryotic cell is a potato cell. In a further aspect, a eukaryotic cell is a cucumber cell. In another aspect, a eukaryotic cell is a millet cell. In another aspect, a eukaryotic cell is a barley cell. In another aspect, a eukaryotic cell is a flax cell. In another aspect, a eukaryotic cell is a watermelon cell. In another aspect, a eukaryotic cell is a blackberry cell. In another aspect, a eukaryotic cell is a strawberry cell. In another aspect, a eukaryotic cell is a cucurbit cell. In another aspect, a eukaryotic cell is a Brassica cell. In another aspect, a eukaryotic cell is a grass cell. In another aspect, a eukaryotic cell is a Setaria cell. In another aspect, a eukaryotic cell is an Arabidopsis cell. In a further aspect, a eukaryotic cell is an algae cell.

In one aspect, a plant cell is an epidermal cell. In another aspect, a plant cell is a stomata cell. In another aspect, a plant cell is a trichome cell. In another aspect, a plant cell is a root cell. In another aspect, a plant cell is a leaf cell. In another aspect, a plant cell is a callus cell. In another aspect, a plant cell is a protoplast cell. In another aspect, a plant cell is a pollen cell. In another aspect, a plant cell is an ovary cell. In another aspect, a plant cell is a floral cell. In another aspect, a plant cell is a meristematic cell. In another aspect, a plant cell is an endosperm cell. In another aspect, a plant cell does not comprise reproductive material and does not mediate the natural reproduction of the plant. In another aspect, a plant cell is a somatic plant cell.

Additional provided plant cells, tissues and organs can be from seed, fruit, leaf, cotyledon, hypocotyl, meristem, embryos, endosperm, root, shoot, stem, pod, flower, inflorescence, stalk, pedicel, style, stigma, receptacle, petal, sepal, pollen, anther, filament, ovary, ovule, pericarp, phloem, and vascular tissue.

In a further aspect, a eukaryotic cell is an animal cell. In another aspect, a eukaryotic cell is an animal cell in culture. In a further aspect, a eukaryotic cell is a human cell. In a further aspect, a eukaryotic cell is a human cell in culture. In a further aspect, a eukaryotic cell is a somatic human cell. In a further aspect, a eukaryotic cell is a cancer cell. In a further aspect, a eukaryotic cell is a mammal cell. In a further aspect, a eukaryotic cell is a mouse cell. In a further aspect, a eukaryotic cell is a pig cell. In a further aspect, a eukaryotic cell is a bovid cell. In a further aspect, a eukaryotic cell is a bird cell. In a further aspect, a eukaryotic cell is a reptile cell. In a further aspect, a eukaryotic cell is an amphibian cell. In a further aspect, a eukaryotic cell is an insect cell. In a further aspect, a eukaryotic cell is an arthropod cell. In a further aspect, a eukaryotic cell is a cephalopod cell. In a further aspect, a eukaryotic cell is an arachnid cell. In a further aspect, a eukaryotic cell is a mollusk cell. In a further aspect, a eukaryotic cell is a nematode cell. In a further aspect, a eukaryotic cell is a fish cell.

### Kits

In an aspect, this disclosure provides a kit for inducing a targeted modification in a target nucleic acid, comprising: (a) a catalytically inactive guided-nuclease, or a nucleic acid encoding the catalytically inactive guided-nuclease; and (b) at least one chemical mutagen.

In an aspect, a kit further comprises a guide nucleic acid. In another aspect, a kit further comprises a guide RNA. In another aspect, a kit further comprises a guide DNA. In another aspect, a kit further comprises a guide nucleic acid comprising DNA and RNA. In another aspect, a kit comprises a nucleic acid encoding a guide nucleic acid. In a further aspect, a kit comprises a nucleic acid encoding a guide RNA. In an aspect, a nucleic acid encoding a catalytically inactive guided-nuclease further comprises a nucleic acid sequence encoding a guide nucleic acid.

In an aspect, a kit comprises a ribonucleoprotein. In an aspect, a kit comprises a ribonucleoprotein comprising a catalytically inactive guided-nuclease and a guide nucleic acid.

In another aspect, a kit comprises at least one bacteria cell. In an aspect, a kit comprises at least one Agrobacterium cell. In an aspect, a kit comprises at least one bacteriophage. In another aspect, a kit comprises bacteria growth media. In another aspect, a kit comprises Agrobacterium growth media.

In an aspect, a kit comprises at least one diluent for reconstituting a catalytically inactive guided-nuclease. In another aspect, a kit comprises at least on diluent for diluting a catalytically inactive guided-nuclease. In another aspect, a kit comprises at least one diluent for reconstituting a chemical mutagen. In another aspect, a kit comprises at least on diluent for diluting a chemical mutagen.

In an aspect, a kit comprises at least one buffer. In another aspect, a kit comprises at least one wash buffer.

In an aspect, a reagent provided in a kit enables the production of a catalytically inactive guided-nuclease in vivo. In an aspect, a reagent provided in a kit enables the production of a catalytically inactive guided-nuclease in vitro. In an aspect, a reagent provided in a kit enables the production of a catalytically inactive guided-nuclease ex vivo.

In an aspect, a reagent provided in a kit enables the introduction of a ribonucleoprotein into a cell. In another aspect, a reagent provided in a kit enables the introduction of a catalytically inactive guided-nuclease, or a nucleic acid encoding a catalytically inactive guided-nuclease, into a cell. In an aspect, a reagent provided in a kit enables the introduction of a guide nucleic acid into a cell.

Reagents, diluents, buffers, and wash buffers can include, without being limiting, water, ethylenediaminetetraacetic acid (EDTA), magnesium, magnesium chloride, magnesium acetate, bovine serum albumin (BSA), sodium, sodium chloride, dimethylsulfoxide (DMSO), glycerol, tris(hydroxymethly)aminomethane (Tris), Tris-HCl, acetic acid, acetate, boric acid, glycine, sodium dodecyl sulfate (SDS), glycine, dithiothreitol (DTT), Triton® X-100, potassium, phosphate, potassium phosphate, potassium acetate, ammonia, sodium bicarbonate, sodium carbonate, citrate, hydrochloric acid, malic acid, maleic acid, ethanol, and methanol.

Also without being limiting, a reagent provided herein can comprise a delivery particle, a delivery vesicle, a viral vector, a nanoparticle, a cationic lipid, a polycation, Agrobacterium, and a protein. Additional non-limiting examples of reagents include Transfectam™, and Lipofectin™. Proteins included in reagents can include, without being limiting, Reverse Transcriptase, RNA Polymerase I, RNA Polymerase II, RNA Polymerase III, RNase A, and RNase H.

In an aspect, a reagent, diluent, buffer, or wash buffer comprises a pH of between 3 and 12. In another aspect, a reagent, diluent, buffer, or wash buffer comprises a pH of between 6 and 8. In another aspect, a reagent, diluent, buffer, or wash buffer comprises a pH of at least 3. In another aspect, a reagent, diluent, buffer, or wash buffer comprises a pH of at least 4. In another aspect, a reagent, diluent, buffer, or wash buffer comprises a pH of at least 5. In another aspect, a reagent, diluent, buffer, or wash buffer comprises a pH of at least 6. In another aspect, a reagent, diluent, buffer, or wash buffer comprises a pH of at least 7. In another aspect, a reagent, diluent, buffer, or wash buffer comprises a pH of at least 8. In another aspect, a reagent, diluent, buffer, or wash buffer comprises a pH of at least 9. In another aspect, a reagent, diluent, buffer, or wash buffer comprises a pH of at least 10. In another aspect, a reagent, diluent, buffer, or wash buffer comprises a pH of at least 11.

In an aspect, a reagent provided in a kit enables the expression of a nucleic acid encoding a catalytically inactive guided-nuclease in vivo. In an aspect, a reagent provided in a kit enables the expression of a nucleic acid encoding a catalytically inactive guided-nuclease in vitro. In an aspect, a reagent provided in a kit enables the expression of a nucleic acid encoding a catalytically inactive guided-nuclease ex vivo.

In an aspect, a kit comprises at least one control expression vector.

### Promoters

In an aspect, a nucleic acid encoding a catalytically inactive guided-nuclease is operably linked to a nucleic acid sequence encoding a promoter. In another aspect, a nucleic acid sequence encoding a guide nucleic acid is operably linked to a nucleic acid sequence encoding a promoter. In an aspect, a promoter is heterologous to an operably linked sequence.

The term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s). In addition to promoters, regulatory elements include, without being limiting, an enhancer, a leader, a transcription start site (TSS), a linker, 5′ and 3′ untranslated regions (UTRs), an intron, a polyadenylation signal, and a termination region or sequence, etc., that are suitable, necessary or preferred for regulating or allowing expression of the gene or transcribable DNA sequence in a cell. Such additional regulatory element(s) can be optional and used to enhance or optimize expression of the gene or transcribable DNA sequence.

As commonly understood in the art, the term “promoter” refers to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of an organism are referred to as “constitutive” promoters. Promoters that drive expression during certain periods or stages of development are referred to as “developmental” promoters. Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of an organism, but with lower levels of expression in other tissue(s) of the organism. Promoters that express within a specific tissue(s) of an organism, with little or no expression in other tissues, are referred to as “tissue-specific” promoters. An “inducible” promoter is a promoter that initiates transcription in response to an environmental stimulus such as heat, cold, drought, light, or other stimuli, such as wounding or chemical application. A promoter can also be classified in terms of its origin, such as being heterologous, homologous, chimeric, synthetic, etc.

As used herein, the term “heterologous” in reference to a promoter is a promoter sequence having a different origin relative to its associated transcribable DNA sequence, coding sequence or gene (or transgene), and/or not naturally occurring in the plant species to be transformed. The term “heterologous” can refer more broadly to a combination of two or more DNA molecules or sequences, such as a promoter and an associated transcribable DNA sequence, coding sequence or gene, when such a combination is man-made and not normally found in nature.

In an aspect, a promoter provided herein is a constitutive promoter. In another aspect, a promoter provided herein is a tissue-specific promoter. In a further aspect, a promoter provided herein is a tissue-preferred promoter. In still another aspect, a promoter provided herein is an inducible promoter. In an aspect, a promoter provided herein is selected from the group consisting of a constitutive promoter, a tissue-specific promoter, a tissue-preferred promoter, and an inducible promoter.

RNA polymerase III (Pol III) promoters can be used to drive the expression of non-protein coding RNA molecules, including guide nucleic acids. In an aspect, a promoter provided herein is a Pol III promoter. In another aspect, a Pol III promoter provided herein is operably linked to a nucleic acid molecule encoding a non-protein coding RNA. In yet another aspect, a Pol III promoter provided herein is operably linked to a nucleic acid molecule encoding a guide RNA. In still another aspect, a Pol III promoter provided herein is operably linked to a nucleic acid molecule encoding a single-guide RNA. In a further aspect, a Pol III promoter provided herein is operably linked to a nucleic acid molecule encoding a CRISPR RNA (crRNA). In another aspect, a Pol III promoter provided herein is operably linked to a nucleic acid molecule encoding a tracer RNA (tracrRNA).

Non-limiting examples of Pol III promoters include a U6 promoter, an H1 promoter, a 5S promoter, an Adenovirus 2 (Ad2) VAI promoter, a tRNA promoter, and a 7SK promoter. See, for example, Schramm and Hernandez, 2002, Genes & Development, 16:2593-2620, which is incorporated by reference herein in its entirety. In an aspect, a Pol III promoter provided herein is selected from the group consisting of a U6 promoter, an H1 promoter, a 5S promoter, an Adenovirus 2 (Ad2) VAI promoter, a tRNA promoter, and a 7SK promoter. In another aspect, a guide RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a single-guide RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a CRISPR RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a tracer RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter.

In an aspect, a promoter provided herein is a Dahlia Mosaic Virus (DaMV) promoter. In another aspect, a promoter provided herein is a U6 promoter. In another aspect, a promoter provided herein is an actin promoter.

Examples describing a promoter that can be used herein include, without limitation, U.S. Pat. No. 6,437,217 (maize RS81 promoter), U.S. Pat. No. 5,641,876 (rice actin promoter), U.S. Pat. No. 6,426,446 (maize RS324 promoter), U.S. Pat. No. 6,429,362 (maize PR-1 promoter), U.S. Pat. No. 6,232,526 (maize A3 promoter), U.S. Pat. No. 6,177,611 (constitutive maize promoters), U.S. Pat. Nos. 5,322,938, 5,352,605, 5,359,142 and 5,530,196 (35S promoter), U.S. Pat. No. 6,433,252 (maize L3 oleosin promoter), U.S. Pat. No. 6,429,357 (rice actin 2 promoter as well as a rice actin 2 intron), U.S. Pat. No. 5,837,848 (root specific promoter), U.S. Pat. No. 6,294,714 (light inducible promoters), U.S. Pat. No. 6,140,078 (salt inducible promoters), U.S. Pat. No. 6,252,138 (pathogen inducible promoters), U.S. Pat. No. 6,175,060 (phosphorus deficiency inducible promoters), U.S. Pat. No. 6,635,806 (gamma-coixin promoter), and U.S. patent application Ser. No. 09/757,089 (maize chloroplast aldolase promoter). Additional promoters that can find use are a nopaline synthase (NOS) promoter (Ebert et al., 1987), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al., Plant Molecular Biology (1987) 9: 315-324), the CaMV 35S promoter (Odell et al., Nature (1985) 313: 810-812), the figwort mosaic virus 35S-promoter (U.S. Pat. Nos. 6,051,753; 5,378,619), the sucrose synthase promoter (Yang and Russell, Proceedings of the National Academy of Sciences, USA (1990) 87: 4144-4148), the R gene complex promoter (Chandler et al., Plant Cell (1989) 1: 1175-1183), and the chlorophyll a/b binding protein gene promoter, PC1SV (U.S. Pat. No. 5,850,019), and AGRtu.nos (GenBank Accession V00087; Depicker et al., Journal of Molecular and Applied Genetics (1982) 1: 561-573; Bevan et al., 1983) promoters.

Promoter hybrids can also be used and constructed to enhance transcriptional activity (see U.S. Pat. No. 5,106,739), or to combine desired transcriptional activity, inducibility and tissue specificity or developmental specificity. Promoters that function in plants include but are not limited to promoters that are inducible, viral, synthetic, constitutive, temporally regulated, spatially regulated, and spatio-temporally regulated. Other promoters that are tissue-enhanced, tissue-specific, or developmentally regulated are also known in the art and envisioned to have utility in the practice of this disclosure.

### Transformation/Transfection

Any method provided herein can involve transient transfection or stable transformation of a cell of interest (e.g., a eukaryotic cell, a prokaryotic cell). In an aspect, a nucleic acid molecule encoding a catalytically inactive guided-nuclease is stably transformed. In another aspect, a nucleic acid molecule encoding a catalytically inactive guided-nuclease is transiently transfected. In an aspect, a nucleic acid molecule encoding a guide nucleic acid is stably transformed. In another aspect, a nucleic acid molecule encoding a guide nucleic acid is transiently transfected.

Numerous methods for transforming cells with a recombinant nucleic acid molecule or construct are known in the art, which can be used according to methods of the present application. Any suitable method or technique for transformation of a cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants.

In an aspect, a method comprises providing a cell with a catalytically inactive guided-nuclease, or a nucleic acid encoding the catalytically inactive guided-nuclease, via Agrobacterium-mediated transformation. In an aspect, a method comprises providing a cell with a catalytically inactive guided-nuclease, or a nucleic acid encoding the catalytically inactive guided-nuclease, via polyethylene glycol-mediated transformation. In an aspect, a method comprises providing a cell with a catalytically inactive guided-nuclease, or a nucleic acid encoding the catalytically inactive guided-nuclease, via biolistic transformation. In an aspect, a method comprises providing a cell with a catalytically inactive guided-nuclease, or a nucleic acid encoding the catalytically inactive guided-nuclease, via liposome-mediated transfection. In an aspect, a method comprises providing a cell with a catalytically inactive guided-nuclease, or a nucleic acid encoding the catalytically inactive guided-nuclease, via viral transduction. In an aspect, a method comprises providing a cell with a catalytically inactive guided-nuclease, or a nucleic acid encoding the catalytically inactive guided-nuclease, via use of one or more delivery particles. In an aspect, a method comprises providing a cell with a catalytically inactive guided-nuclease, or a nucleic acid encoding the catalytically inactive guided-nuclease, via microinjection. In an aspect, a method comprises providing a cell with a catalytically inactive guided-nuclease, or a nucleic acid encoding the catalytically inactive guided-nuclease, via electroporation.

In an aspect, a method comprises providing a cell with a guide nucleic acid, or a nucleic acid encoding the guide nucleic acid, via Agrobacterium-mediated transformation. In an aspect, a method comprises providing a cell with a guide nucleic acid, or a nucleic acid encoding the guide nucleic acid, via polyethylene glycol-mediated transformation. In an aspect, a method comprises providing a cell with a guide nucleic acid, or a nucleic acid encoding the guide nucleic acid, via biolistic transformation. In an aspect, a method comprises providing a cell with a guide nucleic acid, or a nucleic acid encoding the guide nucleic acid, via liposome-mediated transfection. In an aspect, a method comprises providing a cell with a guide nucleic acid, or a nucleic acid encoding the guide nucleic acid, via viral transduction. In an aspect, a method comprises providing a cell with a guide nucleic acid, or a nucleic acid encoding the guide nucleic acid, via use of one or more delivery particles. In an aspect, a method comprises providing a cell with a guide nucleic acid, or a nucleic acid encoding the guide nucleic acid, via microinjection. In an aspect, a method comprises providing a cell with a guide nucleic acid, or a nucleic acid encoding the guide nucleic acid, via electroporation.

In an aspect, a ribonucleoprotein is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

Other methods for transformation, such as vacuum infiltration, pressure, sonication, and silicon carbide fiber agitation, are also known in the art and envisioned for use with any method provided herein.

Methods of transforming cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell with any of the nucleic acid molecules provided herein.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

Delivery vehicles, vectors, particles, nanoparticles, formulations and components thereof for expression of one or more elements of a nucleic acid molecule or a protein are as used in WO 2014/093622 (PCT/US2013/074667). In an aspect, a method of providing a nucleic acid molecule or a protein to a cell comprises delivery via a delivery particle. In an aspect, a method of providing a nucleic acid molecule or a protein to a cell comprises delivery via a delivery vesicle. In an aspect, a delivery vesicle is selected from the group consisting of an exosome and a liposome. In an aspect, a method of providing a nucleic acid molecule or a protein to a cell comprises delivery via a viral vector. In an aspect, a viral vector is selected from the group consisting of an adenovirus vector, a lentivirus vector, and an adeno-associated viral vector. In another aspect, a method providing a nucleic acid molecule or a protein to a cell comprises delivery via a nanoparticle. In an aspect, a method providing a nucleic acid molecule or a protein to a cell comprises microinjection. In an aspect, a method providing a nucleic acid molecule or a protein to a cell comprises polycations. In an aspect, a method providing a nucleic acid molecule or a protein to a cell comprises a cationic oligopeptide.

In an aspect, a delivery particle is selected from the group consisting of an exosome, an adenovirus vector, a lentivirus vector, an adeno-associated viral vector, a nanoparticle, a polycation, and a cationic oligopeptide. In an aspect, a method provided herein comprises the use of one or more delivery particles. In another aspect, a method provided herein comprises the use of two or more delivery particles. In another aspect, a method provided herein comprises the use of three or more delivery particles.

Suitable agents to facilitate transfer of proteins, nucleic acids, mutagens and ribonucleoproteins into a plant cell include agents that increase permeability of the exterior of the plant or that increase permeability of plant cells to oligonucleotides, polynucleotides, proteins, or ribonucleoproteins. Such agents to facilitate transfer of the composition into a plant cell include a chemical agent, or a physical agent, or combinations thereof. Chemical agents for conditioning includes (a) surfactants, (b) an organic solvents or an aqueous solutions or aqueous mixtures of organic solvents, (c) oxidizing agents, (e) acids, (f) bases, (g) oils, (h) enzymes, or combinations thereof.

Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions), Naturally derived or synthetic oils with or without surfactants or emulsifiers can be used, e.g., plant-sourced oils, crop oils (such as those listed in the 9^{th} Compendium of Herbicide Adjuvants, publicly available on line at www.herbicide.adjuvants.com) can be used, e.g., paraffinic oils, polyol fatty acid esters, or oils with short-chain molecules modified with amides or polyamines such as polyethyleneimine or N-pyrrolidine.

Examples of useful surfactants include sodium or lithium salts of fatty acids (such as tallow or tallowamines or phospholipids) and organosilicone surfactants. Other useful surfactants include organosilicone surfactants including nonionic organosilicone surfactants, e.g., trisiloxane ethoxylate surfactants or a silicone polyether copolymer such as a copolymer of polyalkylene oxide modified heptamethyl trisiloxane and allyloxypolypropylene glycol methylether (commercially available as Silwet® L-77).

Useful physical agents can include (a) abrasives such as carborundum, corundum, sand, calcite, pumice, garnet, and the like, (b) nanoparticles such as carbon nanotubes or (c) a physical force. Carbon nanotubes are disclosed by Karn et al. (2004) Am. Chem. Soc, 126 (22):6850-6851. Liu et al. (2009) Nano Lett, 9(3): 1007-1010, and Khoclakovskaya et al. (2009) ACS Nano, 3(10):3221-3227. Physical force agents can include heating, chilling, the application of positive pressure, or ultrasound treatment. Embodiments of the method can optionally include an incubation step, a neutralization step (e.g., to neutralize an acid, base, or oxidizing agent, or to inactivate an enzyme), a rinsing step, or combinations thereof. The methods of the invention can further include the application of other agents which will have enhanced effect due to the silencing of certain genes. For example, when a polynucleotide is designed to regulate genes that provide herbicide resistance, the subsequent application of the herbicide can have a dramatic effect on herbicide efficacy.

Agents for laboratory conditioning of a plant cell to permeation by polynucleotides include, e.g., application of a chemical agent, enzymatic treatment, heating or chilling, treatment with positive or negative pressure, or ultrasound treatment. Agents for conditioning plants in a field include chemical agents such as surfactants and salts.

In an aspect, a catalytically inactive guided-nuclease, or a nucleic acid encoding the catalytically inactive guided-nuclease, is provided to a cell in vivo. In an aspect, a catalytically inactive guided-nuclease, or a nucleic acid encoding the catalytically inactive guided-nuclease, is provided to a cell in vitro. In an aspect, a catalytically inactive guided-nuclease, or a nucleic acid encoding the catalytically inactive guided-nuclease, is provided to a cell ex vivo.

In an aspect, a guide nucleic acid, or a nucleic acid encoding the guide nucleic acid, is provided to a cell in vivo. In an aspect, a guide nucleic acid, or a nucleic acid encoding the guide nucleic acid, is provided to a cell in vitro. In an aspect, a guide nucleic acid, or a nucleic acid encoding the guide nucleic acid, is provided to a cell ex vivo.

In an aspect, a target nucleic acid is contacted by a catalytically inactive guided-nuclease in vivo. In an aspect, a target nucleic acid is contacted by a catalytically inactive guided-nuclease ex vivo. In an aspect, a target nucleic acid is contacted by a catalytically inactive guided-nuclease in vitro. In an aspect, a target nucleic acid is contacted by a guide nucleic acid molecule in vivo. In an aspect, a target nucleic acid is contacted by a guide nucleic acid molecule ex vivo. In an aspect, a target nucleic acid is contacted by a guide nucleic acid molecule in vitro.

In an aspect, a target nucleic acid is contacted by a ribonucleoprotein and mutagen in vivo. In an aspect, a target nucleic acid is contacted by a ribonucleoprotein and mutagen ex vivo. In an aspect, a target nucleic acid is contacted by a ribonucleoprotein and mutagen in vitro.

Recipient plant cell or explant targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a stomata cell, a trichome cell, a root hair cell, a storage root cell, or a tuber cell. In another aspect, this disclosure provides a protoplast. In another aspect, this disclosure provides a plant callus cell. Any cell from which a fertile plant can be regenerated is contemplated as a useful recipient cell for practice of this disclosure. Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U.S. Patent Application Publication 2004/0216189, all of which are incorporated herein by reference. Transformed explants, cells or tissues can be subjected to additional culturing steps, such as callus induction, selection, regeneration, etc., as known in the art. Transformed cells, tissues or explants containing a recombinant DNA insertion can be grown, developed or regenerated into transgenic plants in culture, plugs or soil according to methods known in the art. In one aspect, this disclosure provides plant cells that are not reproductive material and do not mediate the natural reproduction of the plant. In another aspect, this disclosure also provides plant cells that are reproductive material and mediate the natural reproduction of the plant. In another aspect, this disclosure provides plant cells that cannot maintain themselves via photosynthesis. In another aspect, this disclosure provides somatic plant cells. Somatic cells, contrary to germline cells, do not mediate plant reproduction. In one aspect, this disclosure provides a non-reproductive plant cell.

In an aspect, a method further comprises regenerating a plant from a cell comprising a targeted modification.

### Plant Breeding

Plants derived from methods or kits provided herein can also be subject to additional breeding using one or more known methods in the art, e.g., pedigree breeding, recurrent selection, mass selection, and mutation breeding. Modifications produced via methods provided herein can be introgressed into different genetic backgrounds and selected for via genotypic or phenotypic screening.

Pedigree breeding starts with the crossing of two genotypes, such as a first plant comprising a modification and another plant lacking the modification. If the two original parents do not provide all the desired characteristics, other sources can be included in the breeding population. In the pedigree method, superior plants are self-pollinated and selected in successive filial generations. In the succeeding filial generations the heterozygous condition gives way to homogeneous varieties as a result of self-fertilization and selection. Further, modifications that are not selected for, for example off-target modifications are lost. Typically in the pedigree method of breeding, five or more successive filial generations of self-pollination and selection is practiced: F₁ to F₂; F₂ to F₃; F₃ to F₄; F₄ to F₅, etc. After a sufficient amount of inbreeding, successive filial generations will serve to increase seed of the developed variety. The developed variety may comprise homozygous alleles at about 95% or more of its loci.

In addition to being used to create a backcross conversion, backcrossing can also be used in combination with pedigree breeding. Backcrossing can be used to transfer one or more specifically desirable traits from one variety, the donor parent, to a developed variety called the recurrent parent, which has overall good agronomic characteristics yet lacks that desirable trait or traits. However, the same procedure can be used to move the progeny toward the genotype of the recurrent parent but at the same time retain many components of the non-recurrent parent by stopping the backcrossing at an early stage and proceeding with self-pollination and selection. For example, a first plant variety may be crossed with a second plant variety to produce a first generation progeny plant. The first generation progeny plant may then be backcrossed to one of its parent varieties to create a BC₁ or BC₂. Progenies are self-pollinated and selected so that the newly developed variety has many of the attributes of the recurrent parent and yet several of the desired attributes of the non-recurrent parent. This approach leverages the value and strengths of the recurrent parent for use in new plant varieties.

Recurrent selection is a method used in a plant breeding program to improve a population of plants. The method entails individual plants cross-pollinating with each other to form progeny. The progeny are grown and the progeny comprising a desired modification are selected by any number of selection methods, which include individual plant, half-sibling progeny, full-sibling progeny and self-pollinated progeny. The selected progeny are cross-pollinated with each other to form progeny for another population. This population is planted and again plants comprising a desired modification are are selected to cross pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic line. A synthetic line is the resultant progeny formed by the intercrossing of several selected varieties.

Mass selection is another useful technique when used in conjunction with molecular marker enhanced selection. In mass selection, seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Also, instead of self-pollination, directed pollination could be used as part of the breeding program.

Methods and kits provided herein can improve the agronomic characteristics of a plant. As used herein, the term “agronomic characteristics” refers to any agronomically important phenotype that can be measured. Non-limiting examples of agronomic characteristics include floral meristem size, floral meristem number, ear meristem size, shoot meristem size, root meristem size, tassel size, ear size, greenness, yield, growth rate, biomass, fresh weight at maturation, dry weight at maturation, number of mature seeds, fruit yield, seed yield, total plant nitrogen content, nitrogen use efficiency, resistance to lodging, plant height, root depth, root mass, seed oil content, seed protein content, seed free amino acid content, seed carbohydrate content, seed vitamin content, seed germination rate, seed germination speed, days until maturity, drought tolerance, salt tolerance, heat tolerance, cold tolerance, ultraviolet light tolerance, carbon dioxide tolerance, flood tolerance, nitrogen uptake, ear height, ear width, ear diameter, ear length, number of internodes, carbon assimilation rate, shade avoidance, shade tolerance, mass of pollen produced, number of pods, resistance to herbicide, resistance to insects and disease resistance.

In an aspect, this disclosure provides a method of providing a plant with an improved agronomic characteristic, comprising: (a) providing to a first plant: (i) a catalytically inactive guided-nuclease or a nucleic acid encoding the catalytically inactive guided-nuclease; (ii) at least one guide nucleic acid or a nucleic acid encoding the guide nucleic acid, where the at least one guide nucleic acid forms a complex with the catalytically inactive guided-nuclease, where the at least one guide nucleic acid hybridizes with a target nucleic acid molecule in a genome of the plant, and wherein the target nucleic acid comprises a protospacer adjacent motif (PAM) site; and (iii) at least one mutagen; where at least one modification is induced in the target nucleic acid molecule; (b) generating at least one progeny plant from the first plant; and (c) selecting at least one progeny plant comprising the at least one modification and the improved agronomic characteristic.

### Chemotherapy

Many chemotherapy treatments rely on mutagens to induce mutations in undesirable cells, which due to a reduced ability to repair DNA damage can lead to death of the undesirable cells. As a non-limiting example, undesirable cells include pre-cancerous cells, cancer cells, and tumor cells.

Methods and kits provided herein can be used to reduce the amount or concentration of mutagen required to induce death in undesirable cells.

In an aspect, a method or kit provided herein reduces the amount of mutagen needed to kill an undesirable cell by targeting an essential gene of the undesirable cell. As used herein, an “essential gene” refers to any gene that is critical or required for the survival of the undesirable cell. In an aspect, a modification of an essential gene is lethal to the undesirable cell.

In an aspect, a method or kit provided herein induces death of an undesirable cell by using an at least 1% lower concentration of a mutagen as compared to using the mutagen without a catalytically inactive guided-nuclease. In an aspect, a method or kit provided herein induces death of an undesirable cell by using an at least 5% lower concentration of a mutagen as compared to using the mutagen without a catalytically inactive guided-nuclease. In an aspect, a method or kit provided herein induces death of an undesirable cell by using an at least 10% lower concentration of a mutagen as compared to using the mutagen without a catalytically inactive guided-nuclease. In an aspect, a method or kit provided herein induces death of an undesirable cell by using an at least 25% lower concentration of a mutagen as compared to using the mutagen without a catalytically inactive guided-nuclease. In an aspect, a method or kit provided herein induces death of an undesirable cell by using an at least 50% lower concentration of a mutagen as compared to using the mutagen without a catalytically inactive guided-nuclease. In an aspect, a method or kit provided herein induces death of an undesirable cell by using an at least 75% lower concentration of a mutagen as compared to using the mutagen without a catalytically inactive guided-nuclease.

### EXAMPLES

### Example 1. Escherichia coli rpoB/Rif^{r} assay system

This example describes the Escherichia coli rpoB gene and Rifampicin-resistant (Rif^{r}) mutations that map to rpoB as a system to characterize mutation rates and mutation types induced by chemical mutagens.

The E. coli K12 (strain MG1655) rpoB gene (SEQ ID NO: 1) encodes a subunit of the RNA polymerase complex (SEQ ID NO: 2) and is the target of the antibiotic rifampicin, a bacterial transcription inhibitor. A unique feature of the rpoB gene is that at least 69 nucleotide substitutions in 24 amino acid codons can confer the Rif^{r} phenotype to E. coli. This makes rpoB a useful target for screening and analyzing the type and frequency of nucleotide changes (e.g., additions, deletions, and substitutions induced by mutagens (reviewed in Garibyan et al., 2003, DNA Repair, 2:593-608). Furthermore, a majority of mutations that confer Rifampicin resistance (>90%) map to a relatively small, 268-nucleotide long, region of the rpoB gene. This allows PCR amplification with a single pair of oligonucleotide primers, followed by sequencing, which permits rapid analysis of numerous potential mutations. The aforementioned 268-bp fragment of the E. coli K12 rpoB gene is set forth as SEQ ID NO: 3. It should be noted that as an essential gene, E. coli does not tolerate frameshift or nonsense mutations in the rpoB locus. However, mutants comprising short, in-frame deletions (up to five amino acids) have been found, but at a much lower frequency (see, for example, Jin and Gross, J Mol Biol. 202:45-58 (1988)).

EMS Induced Mutations at rpoB:

The chemical mutagen EMS (ethyl methanesulfonate) selectively alkylates guanine bases causing DNA-polymerase to favor placing a thymine residue over a cytosine residue opposite to the O-6-ethyl guanine during DNA replication, which results in a G to A transition mutation. A majority (70% to 99%) of the modifications observed in EMS-mutated populations are G:C→A:T base pair transitions. A multitude of studies have analyzed the effect of EMS treatment on the rpoB gene, the most comprehensive of which is the study carried out by Garibyan et. al. (DNA Repair 2:593-608 (2003)). A total of 40 mutations at eight sites within a 268-bp stretch (SEQ ID NO: 3) of the rpoB gene were identified in this report; all 40 mutations were G:C→A:T transitions. An additional five G:C sites not found in the 40 EMS mutants from Garibyan et al., but found in other Rif^{r} mutants from other treatments or ‘spontaneous Rif^{r}’ have also been reported (see, for example, Garibyan et. al.). The total number of available G:C EMS target sites within the rpoB locus that result in a Rif^{r} phenotype is 13. These sites are listed below in Table 1. Mutations at positions 1546 and 1592 of SEQ ID NO: 1, resul in D516N and S531F substitutions, respectively, and account for more than half (22/40) of the sequenced mutations found by Garibyan et. al. Finally, Garibyan et. al. describe an additional 49 mutations within the 268-bp fragment (SEQ ID NO: 3) which are transversions (e.g., A:T→G:C; A:T→T:A; A:T→C:G; G:C→T:A; G:C→C:G) that also result in the Rif^{r} phenotype.

TABLE 1
Known G:C to A:T mutations within the 268-bp rpoB
fragment (SEQ ID NO: 3) and resultant amino acid substitutions
that lead to Rif^{r} phenotype.

| Nucleotide position | | Resulting amino acid substitution |
|---|---|---|
| of the rpoB gene | Nucleotide | within the rpoB protein |
| (SEQ ID NO: 1) | Change | (SEQ ID NO: 2) |
| 1520 | G to A | G507D |
| 1535 | C to T | S512F |
| 1546 | G to A | D516N |
| 1565 | C to T | S522F |
| 1576 | C to T | H526Y |
| 1585 | C to T | R529C |
| 1586 | G to A | R529H |
| 1592 | C to T | S531F |
| 1595 | C to T | A532V |
| 1600 | G to A | G534S |
| 1601 | G to A | G534D |
| 1691 | C to T | P564E |
| 1721 | C to T | S574F |

### Example 2. Validating Cas9 and Cpf1 Guide RNAs Targeting rpoB

rpoB Target Sites for the RNA-Guided Nucleases Cas9 and Cpf1:

Streptococcus pyogenes Cas9 (SpCas9) and Lachnospiraceae bacterium Cpf1 (LbCpf1) are endonucleases that can be directed to a target locus near a protospacer adjacent motif (PAM) via hybridization between an associated guide RNA (gRNA) and the target site. Once hybridized, the endonucleases carry out dsDNA cleavage at the target site. The 268-bp fragment within the rpoB gene (SEQ ID NO: 3) was investigated for the presence of SpCas9 and LbCpf1 PAM sites. Two target sites for LbCpf1 were identified and these were designated rpoB-1540 (SEQ ID NO:4) and rpoB-1578 (SEQ ID NO: 5). See Table 2. Three target sites were identified for SpCas9, designated rpoB-1526 (SEQ ID NO: 6), rpoB-1599 (SEQ ID NO:7) and rpoB-1605 (SEQ ID NO:8). See Table 3. Expression vectors encoding appropriate guide RNAs were generated for each target site. As a control, an expression vector encoding guide RNAs targeting the corn Zm7.1 locus (a sequence not present in the E. coli genome) were also generated. The Cpf1 target site within Zm7.1 is set forth as SEQ ID NO:9, and the Cas9 target site within Zm7.1 is set forth as SEQ ID NO:10.

TABLE 2
LbCpf1 guide RNA target sites

| | | | Nucleotide |
|---|---|---|---|
| | | | positions |
| LbCpf1 | | | within the |
| Target | | Target sequence 3′ | rpoB gene |
| site | PAM | to PAM | (SEQ ID NO: 1) |
| rpoB-1540 | TTTA | TGGACCAGAACAACCCGCT | 1544-1568 |
| | | GTCTGA (SEQ ID NO: 4) | |
| rpoB-1578 | TTTG | TGCGTAATCTCAGACAGC | 1554-1577 |
| | | GGTTGG (SEQ ID NO: 5) | |
| Lb-Zm7.1 | TTTA | GTATAATATGATGGCAT | None |
| | | GCCCTC (SEQ ID NO: 9) | |

TABLE 3
SpCas9 guide RNA target sites

| | | | Nucleotide |
|---|---|---|---|
| | | | positions |
| SpCas9 | | | within the |
| Target | | Target sequence 5′ | rpoB gene |
| site | PAM | to PAM | (SEQ ID NO: 1) |
| rpoB- | TGG | TTGTTCTGGTCCATAAACTG | 1530-1554 |
| 1526 | | AGACAGC (SEQ ID NO: 6) | |
| rpoB- | CGG | GCACAAACGTCGTATCTCC | 1575-1598 |
| 1599 | | GCACT (SEQ ID NO: 7) | |
| rpoB- | AGG | CGTCGTATCTCCGCACTC | 1582-1604 |
| 1605 | | GGCCC (SEQ ID NO: 8) | |
| Sp- | TGG | GCCGGCCAGCATTTG | None |
| Zm7.1 | | AAACA (SEQ ID NO: 10) | |

pGUIDE Vectors:

Three LbCpf1 pGUIDE vectors were created: pGUIDE-Lb-rpoB-1540, pGUIDE-Lb-rpoB1578 and the control vector pGUIDE-Lb-Zm7.1. The vectors comprise a guide RNA expression cassette comprising: a synthetic promoter P-J23119 (SEQ ID NO: 11) operably linked to a 19-nucleotide DNA sequence encoding the crRNA sequence (SEQ ID NO: 12); a 23- to 25-nucleotide spacer DNA sequence targeting either rpoB-1540 (SEQ ID NO: 4) or rpoB-1578 sites (SEQ ID NO: 5) or Zm7.1 (SEQ ID NO: 9); followed by a DNA sequence encoding the 19-nucleotide crRNA sequence (SEQ ID NO:12) and a T7 termination sequence (see US20180092364-0005).

Four SpCas9 pGUIDE vectors were generated: pGUIDE-Sp-rpoB-1526, pGUIDE-Sp-rpoB1599, pGUIDE-Sp-rpoB1605 and control pGUIDE-Sp-Zm7.1. Each vector comprises a guide RNA expression cassette with a synthetic promoter P-J23119 (SEQ ID NO: 11) operably linked to a spacer sequence targeting one of the four target sites rpoB-1526 (SEQ ID NO: 6), rpoB-1599 (SEQ ID NO: 7), rpoB-1605 (SEQ ID NO: 8), or Zm7.1 (SEQ ID NO: 11) followed by a 103-nucleotide DNA sequence encoding the Cas9 guide RNA sequence (SEQ ID NO:13) and a T7 termination sequence (see US20180092364-0005).

Each Cas9 and Cpf1 pGUIDE vector also comprised an expression cassette for a selectable marker conferring resistance to the antibiotic Spectinomycin (Speck) and pCDF replication origin.

pNUCLEASE (pNUC) Vectors:

Four pNUC vectors: pNUC-cys-free LbCpf1, pNUC-dLbCpf1, pNUC-Cas9, pNUC-dCas9 were generated by standard cloning techniques and are described below:

(1) pNUC cys-freeLbCpf1 vector comprises an expression cassette for the cys-free LbCpf1 nuclease. The protein sequence of cys-free LbCpf1 is set forth as SEQ ID NO: 25. The cys-free LbCpf1 nucleotide sequence was optimized for expression in E. coli (SEQ ID NO: 14) and fused to a DNA sequence encoding a Nuclear Localization Signal (NLS1) (SEQ ID NO: 15) at the 5′ end and a sequence encoding NLS2 at the 3′end (SEQ ID NO: 16). A nucleotide sequence encoding a histidine tag (SEQ ID NO: 17) was introduced at the 5′ end of NLS1-cys-free LbCpf1-NLS2. The nucleotide sequence encoding the fusion protein was operably linked to a regulatory sequence comprising the E. coli P-tac promoter, the bacteriophage T7 gene 10 leader sequence, and a ribosome binding site (SEQ ID NO: 18) (see Olins and Rangwala, J Biol Chem, 264:16973-16976 (1989)).
(2) pNUC-dLbCpf1 was created by replacing the sequence encoding the ORF of cys-free LbCpf1 within pNUC cys-free LbCpf1 with a DNA sequence encoding a deadLbCpf1 (dLbCpf1) (SEQ ID NO: 19). As compared to LbCpf1 protein, the dLbCpf1 protein comprises an aspartic acid to alanine amino acid substitution at position 832, and a glutamic acid to alanine amino acid substitution at position 925. The sequence of dLbCpf1 protein is set forth as SEQ ID NO:24. These substitutions have been shown to result in the complete abolishment of DNA cleavage activity of LbCpf1 (see Zetsche et al., Cell, 163:759-771 (2015); and Yamano et al., Mol Cell, 67:633-645 (2017)).
(3) pNUC-SpCas9 vector was created by replacing the sequence encoding the ORF of cys-free LbCpf1 within cys-free pNUC LbCpf1 with the SpCas9 gene sequence (SEQ ID NO: 20).
(4) pNUC-dSpCas9 vector was created by replacing the sequence encoding the ORF of cys-free LbCpf1 within pNUC cys-free LbCpf1 with a DNA sequence encoding a deadSpCas9 (dSpCas9) (SEQ ID NO: 21). As compared to SpCas9 protein (SEQ ID NO: 23), dSpCas9 protein (SEQ ID NO:22) has an aspartic acid to alanine amino acid substitution at position 10, and a histidine to alanine amino acid substitution at position 840, which result in complete abolishment of DNA cleavage activity of SpCas9 (see Jinek et. al., Science, 337:816-821 (2012)).

Each pNUC vector also comprised an expression cassette for a selectable marker conferring resistance to the antibiotic chloramphenicol (Cm⁺) and a Co1E1 replication origin.

Validating gRNA Activity in E. coli.

Cui and Bikard had previously investigated the consequence of expressing Cas9 with cognate guide RNAs in E. coli (see Cui and Bikard, Nucleic Acids Research, 44:4243-4251 (2016)). Their analysis showed that co-transformation of an active Cas9 protein with a cognate guide RNA that can target the E. coli chromosome leads to a significant reduction in transformation efficiency since this combination can produce a dsDNA break, which is often lethal in E. coli. The effect is even more pronounced if the target sequence is in an essential gene. Co-transformation/co-expression of an active Cas9 protein with a guide targeting a sequence not present in the E. coli genome is well-tolerated. Similarly, co-expression of acatalytically inactive/dead Cas9 protein, with all guide RNA combinations are non-lethal. They also observed that some combinations can exhibit a growth retardation phenotype, possibly due to a CRISPR-interference effect where the bound inactive Cas9-gRNA complex can act as a block to the proper transcription of an essential gene.

To test whether combinations of different pGUIDEs with active and inactive nucleases exhibit the expected effects described above, pNUC and pGUIDE vectors were co-transformed into KL16 cells (E. coli Genetic Stock Center, Yale), a wild-type recA+ strain, by electroporation. The pNUC and pGUIDE combinations are detailed below in Table 4.

TABLE 4
pNUC and pGUIDE combinations transformed into E. coli.

| | | | Target | CFUs/100 μL |
|---|---|---|---|---|
| Trans- | | | site in | LB (Cm⁺, Spec⁺) |
| formation | pNUC | pGUIDE | E. coli | plates |
| 1 | pNUC- | pGUIDE-Lb- | rpoB gene | 0-2 |
| | cys-free | rpoB-1540 | | |
| 2 | LbCpf1 | pGUIDE-Lb- | rpoB gene | 0-2 |
| | | rpoB-1578 | | |
| 3 | | pGUIDE-Lb- | — | ~300 |
| | | Zm7.1 | | |
| 4 | | pGUIDE-Lb- | rpoB gene | ~200 to 500 |
| | | rpoB-1540 | | |
| 5 | pNUC- | pGUIDE-Lb- | rpoB gene | ~200 to 500 |
| | dLbCpf1 | rpoB-1578 | | |
| 6 | | pGUIDE-Lb- | — | ~200 to 500 |
| | | Zm7.1 | | |
| 7 | | pGUIDE-Sp- | rpoB gene | 0-2 |
| | | rpoB-1526 | | |
| 8 | pNUC- | pGUIDE-Sp- | rpoB gene | 0-2 |
| | SpCas9 | rpoB-1599 | | |
| 9 | | pGUIDE-Sp- | rpoB gene | 0-2 |
| | | rpoB-1605 | | |
| 10 | | pGUIDE-Sp- | — | ~200-300 |
| | | Zm7.1 | | |
| 11 | pNUC- | pGUIDE-Sp- | rpoB gene | ~200-300 |
| | dSpCas9 | rpoB-1526 | | |
| 12 | | pGUIDE-Sp- | rpoB gene | ~200-300 |
| | | rpoB-1599 | | |
| 13 | | pGUIDE-Sp- | rpoB gene | ~200-300 |
| | | rpoB-1605 | | |
| 14 | | pGUIDE-Sp- | — | ~200-300 |
| | | Zm7.1 | | |

“—” refers to no known target in the E. coli genome.

Electrocompetent cells were prepared from E. coli KL16 following standard protocol, and frozen in liquid nitrogen in 250 μL aliquots. Approximately 0.7 μL of pNUC and pGUIDE plasmid DNA combinations (comprising an approximate DNA concentration of 50 to 150 ng/μL) described in Table 2 were added to 30 μL aliquots of the KL16 cells. The cells and DNA were electroporated in 1 mm gap cuvettes, using Bio-Rad GenePulser II using standard settings for E. coli (1.8 kV, 25 μF capacitance, 200 Ohms resistance). Time constants were approximately 4.85-5.05 msecs. Approximately 1 mL of S.O.C. medium was added, mixed, and transferred to culture tubes and shaken in a 37° C. incubator for approximately 90 min at 280 RPM. Then, approximately 20 μL of the mix was diluted with 380 μL of S.O.C. medium (equivalent to 1:20 dilution) and 100 μL was plated on LB agar plates containing the appropriate selection antibiotics (+25 μg/mL chloramphenicol (Cm), +50 μg/mL spectinomycin (Spect)) and incubated overnight at 37° C.

As shown in Table 4, the three pGUIDEs co-transformed with pNUC-dLbCpf1 yielded ˜200-500 uniform colonies on the double selection plates (see Table 4, Transformations 4-6). When pGUIDE-Lb-Zm7.1 was co-transformed with pNUC-LbCpf1, 300 colonies were observed on the selection plates (see Table 4, transformation 3). Both pGUIDEs expressing LbCpf1 guides targeting rpoB, however, yielded 0-2 colonies when co-transformed with pNUC-cys-freeLbCpf1 (see Table 4, Transformations 1-2). These observations indicate that cys-free LbCpf1 is a functional nuclease. These observations are in agreement with observations made by Cui and Bikard and are expected for complexes that can cleave an essential gene such as rpoB.

Similarly, when the four SpCas9 pGUIDEs were co-transformed with catalytically inactive pNUC-dSpCas9, all four plates showed ˜200-300 uniform colonies on the double selection plates (see Table 4, transformations 11-14). Transformation of pGUIDE-Sp-Zm7.1 with the active pNUC-SpCas9 also yielded ˜200-300 uniform colonies on the double selection plates (see Table 4, transformation 10). However, when pGUIDE-Sp-rpoB-1526, pGUIDE-Sp-rpoB-1599, or pGUIDE-Sp-rpoB-1605 were co-transformed with pNUC-SpCas9, 0-2 colonies were recovered (see Table 4, Transformations 7-9). Taken together, these data indicate that all rpoB targeting pGUIDEs enabled cleavage within the rpoB locus when paired with their cognate active nuclease.

### Example 3: Utilizing the rpoB/Rif^{r} Assay to Investigate EMS Induced Mutations in E. coli

To test the rate and spectrum of mutations induced by EMS on rpoB, an experiment was performed where E. coli KL16 cells were co-transformed with pNUC-dSpCas9 and pGUIDE-Sp-Zm7.1; treated with 0.1% or 1% EMS. Resulting Rif^{r} mutations were selected and scored. The rpoB gene fragment (SEQ ID NO: 3) was sequenced from each colony and the predominant mutations were identified. As noted above in Example 2, pGUIDE-Sp-Zm7.1 is not expected to target the E. coli chromosome, and thus serves as a negative control.

Transformation of E. coli:

Approximately 0.7 μL of pNUC-dSpCas9 and pGUIDE-Sp-Zm7.1 plasmid DNAs (approximate concentrations of 50 to 150 ng/μL) were added to 30 μL aliquots of electrocompetent E. coli KL16 cells. The cells and DNA were electroporated in 1 mm gap cuvettes, using Bio-Rad GenePulser II with settings described above in Example 2. Approximately 1 mL of S.O.C. medium was added to the cuvettes, mixed, transferred to culture tubes, and shaken in a 37° C. incubator shaker for ˜90 min at 280 RPM. Approximately 20 μL of the mix was diluted with 380 μL of S.O.C. medium (1:20 dilution), 100 μL was plated on LB plates containing +25 μg/mL Cm, +50 μg/mL Spect antibiotics followed by overnight incubation at 37° C. After overnight growth, the plates contained ˜100-400 uniform single colonies. Four to five single colonies from the plates were picked and mixed in 2.5 mL liquid LB media containing +25 μg/mL Cm, +50 μg/mL Spect. The culture was grown overnight at 37° C. with shaking at 280 RPM. The following day the saturated overnight culture was diluted into 3 mL of fresh LB +25 μg/mL Cm, +50 μg/mL Spect at a 1:20 ratio. After growth for ˜3 hours shaking (280 RPM) at 37° C., the culture was in exponential growth with an A600 absorbance measurement of ˜1. The culture was split into three 1 mL aliquots in Eppendorf tubes, then spun at full speed for one minute. The LB supernatant was removed and the small pellets were resuspended by gentle pipetting into 1 mL PBS and transferred to culture tubes.

EMS Mutagenesis Treatment:

Approximately 1 μL of EMS (Sigma M0880) was added to the first tube (0.1% EMS, final conc.) and approximately 10 μL of EMS was added to the second tube (1% EMS, final conc.). The third tube received no EMS (0% EMS). After mixing, the tubes were incubated at 37° C. with shaking for one hour. The 1 mL cultures were transferred back into Eppendorf tubes and spun at full speed for 1 minute. The pellets were washed once with PBS to remove EMS and resuspended in 1 mL LB+25 μg/mL Cm, +50 μg/mL Spect. These suspensions were diluted 1:20 into 2 mL LB+25 μg/mL Cm, +50 μg/mL Spect and shaken (280 RPM) and incubated at 37° C. for overnight recovery and outgrowth.

Determining Rif^{r} Mutant Counts:

Approximately 100 μL of each overnight culture was plated in duplicate on LB (+25 μg/mL Cm, +50 μg/mL μg/mL Rifampicin) or LB (+50 μg/mL Rifampicin). For the 0.1% and 1% EMS treated cells, 5 μL of the cultures were also plated. Plates were grown overnight at 37° C. followed by a second overnight incubation at 30° C. The number of Rif^{r} colony forming units was counted for each treatment and the average CFU score is provided below in Table 5.

TABLE 5
Rif^{r} CFUs from E. coli transformed with pNUC-dCas9 +
pGUIDE-Sp-Zm7.1 and treated with 0%, 0.1% and 1% EMS.

| | | |
|---|---|---|
| | Rif^{r} CFUs in | Rif^{r} CFUs in |
| Treatment | 100 μL (average) | 5 μL (average) |

| | | | |
|---|---|---|---|
| 0% | EMS | 8 | Not plated |
| 0.1% | EMS | 280 | 18 |
| 1% | EMS | >10⁴ | 1036 |

To determine the total viable count, overnight culture from the 0.1% EMS treatment was diluted 1:10⁶ in PBS and 100 μL was plated on LB+Cm+Spect plates. Plates were grown overnight at 37° C. followed by a second overnight at 30° C. Viable CFU from the 0.1% EMS treatment plates was calculated to be 2.5×10⁹/mL. Assuming that the viable CFU's from the saturated overnight cultures are about the same for all three treatments (˜2.5×10⁹), the mutation rate for each treatment was calculated using the formula: Mutation rate=Rif^{r} CFUs per mL/Viable CFUs per mL. Thus, the spontaneous mutation rate (0% EMS) was ˜80/2.5×10⁹=3.2×10⁻⁸. If the spontaneous mutation rate was set as 1, then 0.1% EMS increased the mutation rate by ˜40×, and 1.0% EMS increased the mutation rate by ˜2600× relative to the spontaneous mutation rate.

Characterizing the Position and Frequency of Mutations by Deep Sequencing:

Ninety-six Rif^{r} colonies from 0.1% and 1% EMS treatment assays were picked and colony PCR was carried out to amplify the 263-nucleotide rpoB fragment. Approximately 20 μL PCRs reactions were carried out in 96 well plates, using forward and reverse primers comprising Illumina barcoded adaptors and designed to amplify a 263-nucleotide rpoB fragment associated with mutations conferring the Rif^{r} phenotype. The success of the reactions was checked by running ˜5 μL of 8-12 samples picked from across the 96 wells and running on e-gels. Each amplicon generated from a single Rif^{r} colony was then processed for Illumina-based deep-sequencing using an Illumina 2X300 MiSeqplatform using the manufacturer's recommended procedure. Between 7000-14000 amplicons were generated from each colony. After obtaining the raw reads, adaptor sequences were removed with the program Cutadapt (Martin, EMBnet.journal, 2011, [S1], v17, n1, p. pp. 10-12, ISSN 2226-6089) and low quality reads are filtered with Trimmomatic (Version 0.36) (Bolger et al., Bioinformatics, 30:2114 (2014)). The program ‘glsearch’ (Pearson, Methods Mol Biol., 132:185-219 (2000)), was used to map reads to the reference rpoB sequence and to detect substitutions and small INDELs. A python script was developed to parse out the mapping results.

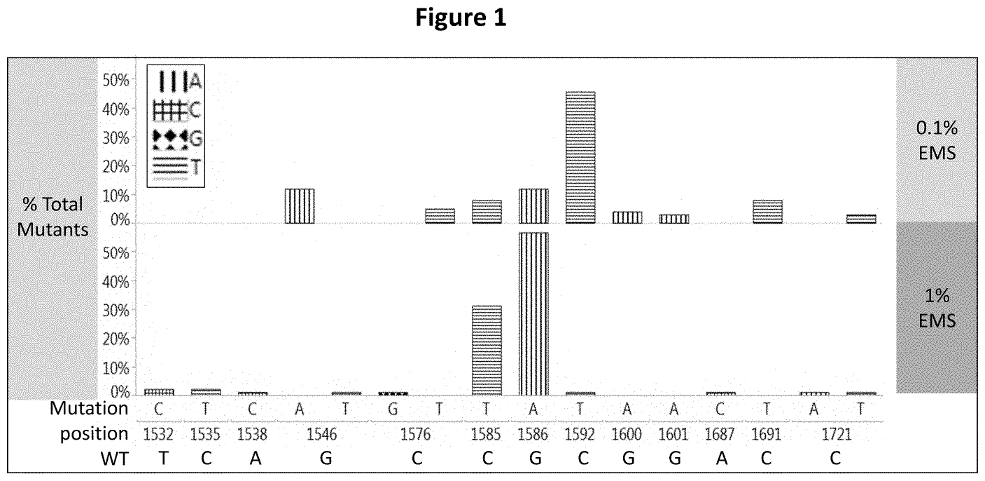

More than 90% of the sequences amplified from each Rif^{r} colony had one predominant sequence with a single point mutation. The predominant mutation identified for each colony and the frequency of occurrence among the sequenced colonies, is described in FIG. 1. As shown in FIG. 1, ten of the thirteen mutations previously described in the art as inducing the Rif^{r} phenotype were identified in this EMS screen. A majority of the EMS mutations were found concentrated between nucleotide positions 1585 and 1600. Most of the mutations where G:C→A:T transition mutations, in agreement with the expected effect of EMS mutagenesis. Three non-G:C→A:T mutations, T1532C, A1538C, and A1687C, were also identified. Whole genome mutagenesis studies have previously reported the occurrence, albeit at low frequencies, of GC→TA, AT→TA, AT→CG, GC→CG and AT→GC type transitions and transversions in EMS treated populations (see, for example, Minoia et al., BMC Research Notes, 3:69 (2010); and Shrasawa et al., Plant Biotechnology, 14:51 (2015)). Four colonies had an in-frame 15 nucleotide deletion resulting in a five amino acid deletion, which had not been previously reported. Few Rif^{r} mutants lacked a mutation in the 268-nucleotide amplicon, which is in agreement with studies in the art. Importantly no INDELs causing a frameshift mutation where observed, which was expected since the rpoB gene is an essential gene.

### Example 4: Targeted EMS Mutagenesis of rpoB Gene in the Presence of Catalytically Inactive RNA-Guided Endonucleases and rpoB Guide RNAs

This example describes an experiment carried out to investigate if EMS-induced mutagenesis can be enhanced in a targeted region by performing EMS mutagenesis in cells transformed with dLbCpf1 or dSpCas9 and cognate gRNAs targeting regions of the rpoB gene.

E. coli KL16 cells were transformed with pNUC and pGUIDE vector combinations described below in Table 6 using the protocol described above in Example 3.

TABLE 6
Rif^{r} CFUs from E. coli cells transformed with pNUC + pGUIDE
vectors followed by treatment with 0.1% EMS.

| Trans- | Type | | | Target | Rif^{r} CFUs/mL | | |
|---|---|---|---|---|---|---|---|
| forma- | of | | | site in E. | (P = plate #) | | |
| tion | treatment | pNUC | pGUIDE | coli | P1 | P2 | P3 |
| 1 | Test | pNUC- | pGUIDE- | rpoB | 11000 | | |
| | | dSpCas9 | Sp-rpoB- | | | | |
| | | | 1526 | | | | |
| 2 | Control | pNUC- | pGUIDE- | none | 1030 | 920 | 1130 |
| | | dSpCas9 | Sp-Zm7.1 | | | | |
| 3 | Test | pNUC- | pGUIDE- | rpoB | 2190 | 1450 | 1480 |
| | | dLbCpf1 | Lb-rpo- | | | | |
| | | | B1578 | | | | |

Three to five uniform double-transformed colonies from each treatment were pooled and grown overnight in LB+25 μg/mL Cm, +50 μg/mL Spect. The overnight culture was diluted 1:20 into fresh antibiotic medium, split in triplicate (for transformations 2 and 3) or duplicate (for transformation 1), re-grown and subsequently treated with 0.1% EMS for ˜50 min, then washed and recovered as described above in Example 3. After overnight growth for recovery, 100 μL from each replicate was plated on LB+25 μg/mL Cm, +50 μg/mL rifampicin (Rif) plates. After two days (day one at 37° C.; day two at 30° C.) the Rif^{r} CFUs for each treatment were scored and are reported in Table 6. For transformation 1, Rif^{r} colonies were counted from a single plate.

Characterizing the Position and Frequency of Mutations:

Individual colonies as well as plate scrapes (pooled colonies) were sequenced. Ninety-two single Rif^{r} colonies were picked from each of the three treatments. The plates were then flooded with PBS, scraped, and spun down. A small aliquot of the pelleted cells was used as the template for the ‘Plate scrape sample’ for each treatment. Amplicon generation, deep-sequencing and rpoB mutation detection was carried out essentially as described above in Example 3. Quality reads were obtained from 71 colonies for treatment 1, 92 colonies for treatment 2, and 89 colonies for treatment 3. Quality reads were also obtained from the Plate scrape samples.

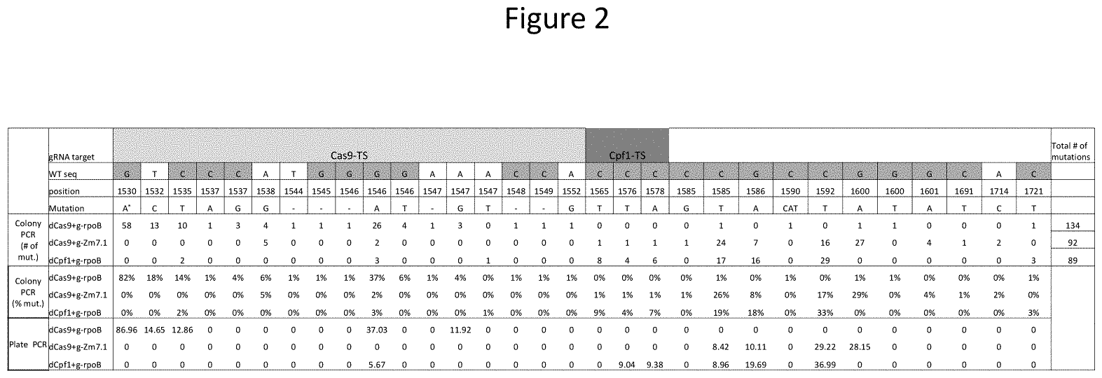

All identified mutations and their frequency of occurrence are provided in FIG. 2. In the control treatment, pNUC-dCas9+pGUIDE-SpZm7.1, 81% of the EMS mutations (71/92) were found concentrated between nucleotide positions 1585 and 1600 in the rpoB gene (SEQ ID NO: 1). These were all canonical G:C→A:T type mutations. This is consistent with Garibyan et al., and the results of 0.1% EMS with untargeted dCas9 described in Example 3. In the dCas9 test treatment (pNUC-dCas9+pGUIDE-Sp-rpoB-1526), 97% of identified mutations (129/134) were observed within the rpoB-1526 guide RNA target region spanning nucleotide positions 1530 to 1554 in the rpoB gene (SEQ ID NO: 1). In contrast, only 8% of the mutations (7/92) identified in the control treatment fell within this region. Of the 129 mutations identified from the dCas9 test treatment and residing within the gRNA target region, 94 were G:C→A:T transitions typically induced by EMS.

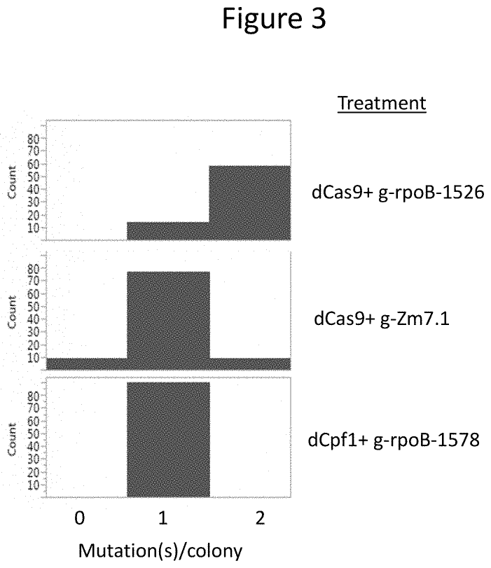

A higher frequency of double mutants were observed in pNUC-dCas9+pGUIDe-Sp-rpoB-1526 test treatment (FIG. 3). Amplicons from 58 out of the 76 Rif^{r} colonies had a silent G1530A mutation as well a second single nucleotide substitution or in-frame INDEL. Twelve types of secondary mutations were identified among the 58 colonies and these are provided in Table 7. Eight of the twelve types of identified second mutations were located within the guide RNA targeted region. Nine of the twelve types of identified second mutations have previously been reported to result in the Rif^{r} phenotype. One novel mutation (cytosine to guanine at position 1537 of SEQ ID NO: 1) resulted in a glutamine to glutamic acid substitution at position 513 (Q513E) of the rpoB protein (SEQ ID NO: 2). This has not been reported in the art though Q513 to arginine, leucine, proline, and lysine substitutions are known to result in Rif^{r} phenotype (see Garibyan et al.). One guanine to adenosine substitution at position 1530 of rpoB (SEQ ID NO:1) Rif^{r} mutant had a 6 nucleotide (2 amino acid) in-frame deletion which has not been previously reported but was observed in amplicons obtained from the treatment described in Example 3. Finally, a novel three nucleotide in-frame insertion between positions 1590 and 1591 of SEQ ID NO: 1 also resulted in a Rif^{r} phenotype.

TABLE 7
Double mutants identified in the pNUC-DCas9 + pGUIDE-Sp-rpoB-
1526 treatment. The Mutation 1 and Mutation 2 columns provide the
identity original nucleotide, the position of the original nucleotide
(in SEQ ID NO: 1) and the identity of the mutated nucleotide.

| # of | Mutation 1 | |
|---|---|---|
| Colonies | (silent) | Mutation 2 |
| 10 | G1530A | C1535T |
| 4 | G1530A | C1537G |
| 4 | G1530A | A1538G |
| 26 | G1530A | G1546A |
| 1 | G1530A | 6 nt in-frame deletion of 1544-1549 |
| 4 | G1530A | G1546T |
| 4 | G1530A | A1547G |
| 1 | G1530A | A1552G |
| 1 | G1530A | C1585T |
| 1 | G1530A | ‘CAT’ insertion between 1590 and 1591 |
| 1 | G1530A | G1600C |
| 1 | G1530A | G1600T |

For the pNUC-dCpf1+pGUIDE-Lb-rpoB1578 treatment, 20% of the identified mutations (18/89) were within the guide RNA target region spanning nucleotide positions 1554 to 1577 of SEQ ID NO: 1. In comparison, in the control treatment only 3% of the mutations (3/92) mapped to this region. Of the 18 identified mutations from the dCpf1 test treatment within the guide RNA target region, 12 were G:C→A:T transitions typically induced by EMS.

The diversity and percentage of reads for each mutation observed from the Plate scrape PCR samples showed similar distributions and rates to those seen from colony PCRs from each treatment (FIG. 2).

These results indicate that using catalytically inactive/dead variants of known CRISPR-associated proteins such as SpCas9 and LbCpf1 paired with a guide RNA targeting a chosen region of a bacterial genome (e.g. the rpoB gene of E. coli) and performing mutagenesis with a DNA mutagen (e.g., EMS) can result in significant enrichment of mutations within the site targeted by the CRISPR-associated protein/guide complex. Furthermore, novel mutations can be recovered that were not part of the selection screen.

### Example 5: Targeted Mutagenesis for Functional Selection

This example describes combining catalytically inactive programmable DNA cleavage enzymes with DNA base modifying chemical mutagens to enrich mutagenesis in targeted regions of the enzyme 5-enolpyruvylshikimate-3-phosphate synthase (EPSPS).

EPSPS is the enzyme that catalyzes the conversion of phosphoenolpyruvate and 3-phosphoshikimate to phosphate and EPSPS. This enzyme is inhibited by the competitive inhibitor glyphosate, which is used widely in agriculture as an herbicide. The structure of EPSPS has been determined and single point mutants within the active site that overcome glyphosate inhibition have been identified. Bacterial screens with E. coli have been developed that allow for selection of improved EPSPS variants in the presence of glyphosate (see Jin et al., Curr. Microbiol., 55:350 (2007)). Variant EPSPS enzymes have been generated by multiple methods, including untargeted methods such as error-prone PCR or targeted approaches that are expensive and require highly-skilled researcher inputs to develop designs and molecular biology skills for saturation mutagenesis libraries.

DNA base modifying chemical mutagens in combination with catalytically inactive CRISPR associated protein/guide RNA complexes can be coupled together with activity selection, such as in a bacterial EPSPS functional selection assay, to enrich mutagenesis to a selected region of the enzyme EPSPS.

Cpf1 or Cas9 gRNAs targeting a specific region of the EPSPS enzyme, such as the residues lining the active site, are designed. In some embodiments, a synthetic EPSPS gene containing PAM sites at the desired location(s) is used. E. coli expressing the EPSPS gene is transformed with dLbCpf1 or dSpCas9 and cognate gRNAs. The transformed cells are subsequently treated with a EMS, and mutagenized cells are placed under selection by glyphosate. Mutations accumulate at higher rates in the targeted region of EPSPS, and when placed under selection by glyphosate the recovery of resistance-conferring mutations derived from the targeted residues is increased.

### Example 6: In Planta Targeted Gene Modification

Random chemical mutagenesis approaches to enhancing genetic diversity in plants requires balancing multiple factors for finding mutations in a candidate gene including, mutation rate, viability and sterility after treatment, population size, and the window of sequence evaluation.

As the mutation rate decreases, the number of individuals screened to find a desired mutation increases exponentially. The local mutation rate induced by DNA base modifying chemical mutagens can be increased by utilizing sequence targeting enzymes (e.g., catalytically inactive RNA-guided endonuclease enzymes such as dCpf1 and dCas9). Once local mutation rates are increased, the number of individuals screened to find a desired mutation is reduced.

To enable this approach, the catalytically inactivated RNA-guided endonucleases and guide RNAs are provided to the nucleus of a plant cell treated with chemical mutagens. The catalytically inactivated RNA-guided endonuclease, gRNA, and EMS are titrated following standard procedures in the art to establish an initial kill-curve analysis for the dose and exposure times leading to a defined mortality (typically, 50% mortality).

Targeted modification can be accomplished in multiple ways, including by expressing a catalytically inactivated RNA-guided endonuclease (e.g., dCpf1, dCas9) within the plant cell, either by co-delivering DNA or mRNA encoding the catalytically inactivated RNA-guided endonuclease or via stable transformation of the plant cells with transgenes encoding the catalytically inactive RNA-guided endonuclease enzymes and/or gRNA. Following expression of the catalytically inactivated RNA-guided endonuclease and gRNA, EMS is applied using standard methods to induce modifications of the target site.

An alternative approach for delivering a catalytically inactivated RNA-guided endonuclease and gRNA complex is to deliver the complex transiently as a ribonucleoprotein, which can be performed on a range of tissue types including leaves, pollen, protoplast, embryos, callus, and others. Following or concurrently with delivery, EMS is applied using standard methods to induce targeted modifications of the target site.

A number of seeds or regenerated plants are grown and screened for mutations in the targeted region using standard methods known in the art.

### Example 7: Increasing Accessibility of DNA-Damaging Chemistries for Therapeutic Treatments

Direct chemical modification of DNA to interfere with normal DNA replication has been shown to be effective in cancer therapy. Cancer cells have relaxed DNA damage sensing/repair capabilities, which helps them achieve high replication rates and also makes them more susceptible to DNA damage.

The replication of damaged DNA increases the probability of cell death and has been the focus for anticancer compound development. The DNA alkylating-like platinum compound Cis-diamminedichloroplatinum(II) (cisplatin) forms DNA adducts with guanine and, to a lesser extent, adenine residues. When two platinum adducts form on adjacent bases on the same DNA strand they form instrastrand crosslinks. These intrastrand crosslinks block DNA replication and cause cell death if not repaired (see Cheung-Ong et. al., Chem. Biol., 20:648-59 (2013)). These therapies are not without side effects and discovery efforts for cisplatin analogs are directed to reducing toxicity in nontargeted tissues (see Bruijnincx and Sadler, Curr. Opin. Chem. Biol., 12:197-206 (2008)).

The compositions and methods described herein may be used to increase the effectiveness of a non-targeted chemical DNA modifying therapeutic agent. Not wishing to be bound by a particular theory, the DNA bases of essential genes can be made more available for chemical modification by the unwinding action of catalytically inactivated RNA-guided endonuclease/guide complexes. The delivery of catalytically inactivated RNA-guided endonuclease/guide complexes to target cancer cells is an active area of development and routes for selective targeting of tumor cells could include oncolytic viruses and microinjection. These routes could be used for selective delivery of catalytically inactivated RNA-guided endonucleases (see Liu et al., J Control Release, 266:17-26 (2017)). The combined effect of selectively unwinding and making available targeted DNA (by exposing the targeted bases from within the more protected dsDNA helix) for chemical modification in cancerous cells may lower the total dosage of DNA modifying chemotherapeutic required to induce cancer cell death. By lowering the total dosage of chemical therapeutic agent required, the adverse toxicity to non-target tissues would be expected to be reduced.

### Example 8: In Vitro DNA Cleavage Activity of Cys-Free LbCpf1

Cysteine residues in a protein are able to form disulfide bridges providing a strong reversible attachment between cysteines. To control and direct the attachment of Cpf1 in a targeted manner the native cysteines are removed. Removal of the cysteines from the protein backbone enables targeted insertion of new cysteine residues to control the placement of these reversible connections by a disulfide linkage. This could be between protein domains or to a particle, such as a gold particle for biolistic delivery. It is unknown whether the nine cysteine residues present in the LbCpf1 protein (WO 2016205711-1150) participate in the DNA cleavage activity. A mutant LbCpf1 protein containing no cysteines was therefore generated and tested for activity. A cys-free LbCpf1 protein was generated containing the following 9 amino acid substitutions: C10L, C175L, C565S, C632L, C805A, C912V, C965S, C1090P, and C1116L.

An in vitro DNase activity assay was developed to investigate the targeted double-stranded (ds) DNA cleavage activity of LbCpf1 and cys-free LbCpf1. The dsDNA Template used in the assay was a 492 bp PCR product (SEQ ID NO: 26) containing the LbCpf1 target site Lb-Zm7.1 that spans the region of nucleotides 269 to 291 in SEQ ID NO: 26 (see Table 8). Cutting activity by LbCpf1 and its cognate Cpf1-Zm7.1 gRNA would result in 197 bp and 295 bp DNA digestion fragments.

The nucleases were expressed and purified from Escherichia coli by standard methods. For this purpose, an E. coli expression vector pNUC-LbCpf1 was created by replacing the sequence encoding the ORF of cys-free LbCpf1 within pNUC cys-freeLbCpf1 vector described in Example 2 with a DNA sequence encoding E. coli codon optimized LbCpf1 (SEQ ID NO: 27). The LbCpf1 guide RNA (SEQ ID NO: 28) comprising the LbCpf1 crRNA sequence and the 23 nucleotide spacer sequence targeting Lb-Zm7.1 was synthesized by standard methods.

TABLE 8
LbCpf1 guide RNA target site

| | | | Nucleotide |
|---|---|---|---|
| | | | positions |
| LbCpf1 | | | within the 491 |
| Target | | Target sequence 3′ | bp template |
| site | PAM | to PAM | (SEQ ID NO: 26) |
| Lb-Zm7.1 | TTTA | GTATAATATGATGGCATG | 269 to 291 |
| | | CCCTC (SEQ ID NO: 9) | |

Typical reactions were carried out in cleavage buffer consisting of 50 mM Tris-HCl (ph7.6), 100 mM NaCl, 10 mM MgCl2, 5 mM DTT. Purified nucleases was pre-diluted to 20 μM in 1× cleavage buffer, and further serial dilutions (typically 1:4) were made in the same buffer. Purified guide RNA was pre-diluted to 20 μM in ddH20, and further serial dilutions (typically 1:4) were made in ddH20. After mixing the Ribonucleoprotein (RNP) components (without substrate DNA), the reactions were incubated at room temperature for 10-15 mins. The reactions were started with the addition of template DNA at concentrations that achieved the RNP:DNA ratio described in Table 9. The reactions were carried out at 37° C. for 45 minutes and quenched with proteinase K treatment at 65° C. for 15 minutes. The samples were then resolved and analyzed on a 2% TBE Agarose gel. Additionally, to quantify nuclease protein concentrations for each assay, aliquots of the samples were resolved and analyzed by SDS-PAGE. Concentrations of proteins was also measured by calculating absorbance at A280 nm.

Visual inspection of agarose gels indicated that greater than 90% cutting was achieved with both LbCpf1 and cys-free LbCpf1 at 45 minutes in assays with 20:1 RNP:DNA ratio. Both enzymes showed partial cutting activity at 45 minutes in assays with 5:1 RNP:DNA ratio (see Table 9). SDS-PAGE analysis confirmed equivalent protein concentrations for assays 1 and 2, 3 and 4. Taken together, the data suggests that cys-free LbCpf1 retains targeted dsDNA cleavage activity.

TABLE 9
DNA Cleavage assay. For targeted dsDNA cleavage, “Yes”
refers to the observation of the 197 bp and 295 bp
DNA fragments on the gel.

| | 491 bp | | | | Targeted |
|---|---|---|---|---|---|
| | Template | | gRNA | | dsDNA |
| | with Zm7.1 | | targeting | RNP:DNA | cleavage of |
| Assay | target site | Nuclease | Zm7.1 | (mol:mol) | template |
| 1 | + | LbCpf1 | + | 20:1 | Yes |
| 2 | + | Cys-free | + | 5:1 | Yes |
| | | LbCpf1 | | | |
| 3 | + | LbCpf1 | + | 20:1 | Yes |
| 4 | + | Cys-free | + | 5:1 | Yes |
| | | LbCpf1 | | | |

### Example 9. Bacillus subtilis upp/5-FU Assay System

This example describes the Bacillus subtilis upp gene and knockout mutations resulting in resistance to 5-fluorouracil (5-FU) as a system to characterize mutation rates and mutation types induced by random mutagenesis.

The B. subtilis (substrain 168) upp gene (SEQ ID NO: 29) encodes an uracil phosphoribosyltransferase, which is a pyrimidine salvage enzyme. This enzyme also converts 5-fluorouracil directly to 5-fluorouridine monophosphate, a potent inhibitor of thymidylate synthetase (see Neuhard, J. Metabolism of nucleotides, nucleosides and nucleobases in microorganisms/edited by A. Munch-Petersen (1983)). Culturing B. subtilis on plates supplemented with 5-FU causes toxicity for all cells expressing a functioning upp gene and selection for cells lacking a functional copy (see Fabret et al., Molecular Microbiology, 46:25-36 (2002)). This makes upp a useful target for detecting low levels of mutagenesis, with a variety of potential mutations (e.g., additions, deletions, substitutions) that will result in selectable loss-of-function. The small size of this gene (630 bp, 210 aa) allows for PCR amplicon sequencing and rapid analysis of numerous potential mutations. It should be noted that the upp gene is inessential when ample uracil is supplied.

Light Induced Mutations at upp:

Mutagenesis has been previously observed resulting from visible light alone (see McGinty and Fowler, Mutation Research/Fundamental and Molecular Mechanisms of Mutagenesis 95, 171-181 (1982)). The most frequently observed mutation is G:C→T:A base pair transversions. Any out-of-frame insertion/deletion mutation in the first 441 nt of the upp gene will result in a premature stop before the C-terminal active site that would result in upp loss-of-function. There are 21 potential G:C→T:A base pair transversions that would result in a premature stop before the C-terminal active site (provided in Table 10).

TABLE 10
Predicted G:C→T:A transversions
within the first 486 nt of upp
resulting in premature stop.

| Resulting | | | |
|---|---|---|---|
| stop codon | WT codon, AA (n) | | |
| TGA | GGA, G (5) | TGC, C (0) | |
| TAA | GAA, E (10) | TCA, S (1) | TAC, Y (2) |
| TAG | GAG, E (2) | TCG, S (1) | |

### Example 10. Generating Cpf1 Expression Strains of B. subtilis str. 168

Identified Upp Target Sites for the RNA-Guided DNA-Binding Protein dLbCpf1:

Five LbCpf1 target sites (SEQ ID NO: 31-35) were identified within the upp gene of B. subtilis (SEQ ID NO: 29). Five target sequences within amyE gene of B. subtilis (SEQ ID NO: 36-40) were chosen as off-target controls. For both sets, target sequences were chosen for their predicted score using DeepCpf1 (Kim, 2018). Also, targets were designed to eliminate effects of CRISPR-induced inhibition (CRISPRi) by targeting the non-template strand (Kim et al., ACS Synthetic Biology, 2017 6(7), 1273-1282, DOI: 10.1021/acssynbio.6b00368).

TABLE 11
LbCpf1 guide RNA target sites
within amyE and upp gene

| | | | | Nucleotide positions |
|---|---|---|---|---|
| | | | | withinthe target |
| | | | | gene amyE- |
| LbCpf1 | | SEQ | | SEQ ID NO: 29 |
| Target | | ID | Target sequence 3′ | upp- |
| site | PAM | NO: | to PAM | SEQ ID NO: 30 |
| amyE-604 | TTTC | 31 | AGATAGGACTGTACTTGTGTATT | 600-574 |
| | | | | (amyE) |
| amyE-1084 | TTTC | 32 | CCCGGGAACCTCACACCATTTCC | 1080-1054 |
| | | | | (amyE) |
| amyE-1734 | TTTA | 33 | CCTGGCTCCAATGATTCGGATTT | 1730-1704 |
| | | | | (amyE) |
| amyE-664 | TTTG | 34 | GCGGCATCAAATCGAAAACCGTC | 660-634 |
| | | | | (amyE) |
| amyE-483 | TTTG | 35 | GAATACTCTTAACCTCATTGGAA | 479-453 |
| | | | | (amyE) |
| upp-563 | ATCT | 36 | AGCGCCGCAATGTAAATAT | 559-533 |
| | | | | (upp) |
| upp-193 | GCAG | 37 | CCTGAACCGGTGTATTGAT | 189-163 |
| | | | | (upp) |
| upp-273 | AAAT | 38 | GCCGTCAACCATTCCCAAT | 269-243 |
| | | | | (upp) |
| upp-71 | ATTC | 39 | CGTATATATGTCAGCTTGT | 67-41 |
| | | | | (upp) |
| upp-134 | AAAT | 40 | GCCATGAGTGTAGCCACTT | 130-104 |
| | | | | (upp) |

Guide Expression Constructs:

Guide RNA expression vectors comprising expression cassettes targeting the upp gene and any gene were created. Each cassette comprised a synthetic, broad-spectrum constitutive promoter driving a series of direct repeats and five 23-bp targeting sequences terminated by a T7 terminator. The 5Xupa gRNA expression cassette is set forth as SEQ ID NO: 41 and the 5XamyE expression cassette is set forth as SEQ ID NO:42. The guide expression cassettes were inserted into pBV070, a modified derivative of pMiniMAD (Patrick and Kearns, Molecular Microbiology 70, 1166-1179 (2008)) between BamHI and EcoRI restriction sites. These plasmids included selectable markers conferring resistance to the antibiotics ampicillin (for E. coli cloning) and erythromycin (for B. subtilis maintenance), origins from pBR322 (for E. coli cloning) and temperature-sensitive pE194 (for B. subtilis maintenance), and a mobilization fragment (mob) to allow bacterial conjugation.

Cas-Protein Expression Construct:

A dLbCpf1 expression plasmid was constructed. This plasmid comprised a dLbCpf1 expression cassette (SEQ ID NO: 43) comprising nuclear localization sequences at either end of the dLbCpf1 coding sequence that is driven by a synthetic, broad-spectrum constitutive promoter and terminated by a T7 terminator. The dLbCpf1 expression plasmid included selectable markers conferring resistance to the antibiotic kanamycin, and origins from pBR322 (for E. coli cloning) and temperature-sensitive pE194 (for B. subtilis maintenance).

Transformation of B. subtilis:

Competent B. subtilis str. 168 were prepared by standard methods.

### Example 11: Targeted Light-Induced Mutagenesis of Upp Gene in the Presence of Catalytically Inactive RNA-Guided Endonucleases and Upp Guide RNAs

To test the rate and spectrum of mutations induced by light-induced mutagenesis, an experiment was performed where B. subtilis str. 168 cells were co-transformed with combinations of guide expression and dLbCpf1 expression plasmids (Table 11).

TABLE 11
Combinations of guide expression and
fusion dLbCpf1 expression plasmids.

| Strain name | dLbCpf1 plasmid | Guide plasmid |
|---|---|---|
| 3554 | pBV035 (dLbCpf1) | pBV054 (5XamyE) |
| 3555 | pBV035 (dLbCpf1) | pBV055 (5Xupp) |

Light-Induced Mutagenesis Treatment:

A single colony for each plasmid combination was inoculated into LB medium supplemented with lincomycin (25 mg/L), kanamycin (5 mg/L) and erythromycin (1 mg/L) and cultured overnight at 30° C. Overnight cultures were diluted 25-fold into fresh selective media and arrayed into 24-well blocks for incubation at 30° C. with agitation and with or without illumination cycling (15 minutes on, 1 hour off) over 24 hours.

Determining 5-FU Resistant Counts:

Cultures were diluted 10-fold into LB and 100 μl were plated in triplicate onto LB agar plates containing 6.5 mg/L 5-FU to quantify resistant CFU. After a 24-hour incubation at 37° C., resistant CFU were counted.

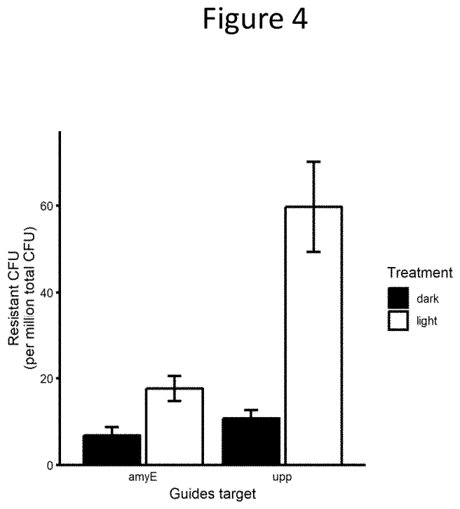

To determine total viable count, treated cultures were further diluted to 105 in LB and plated onto LB agar plates to quantify total CFU. After an overnight incubation at 30° C., total CFU were counted. The results of this experiment are summarized by the rate of resistant CFU provided in FIG. 4. In this experiment, targeting the upp gene resulted in a 3-fold increase in rate of resistant CFU relative to targeting amyE gene. Light cycling resulted in a 5-fold increase in rate of resistant CFU relative to dark treatment.

1. A method of inducing a targeted modification in a target nucleic acid molecule, comprising contacting the target nucleic acid molecule with:
(a) a catalytically inactive guided-nuclease; and
(b) at least one mutagen,
wherein at least one modification is induced in the target nucleic acid molecule.

2. (canceled)

3. A method of increasing the mutation rate in a targeted region of a nucleic acid molecule, comprising contacting the nucleic acid molecule with:
(a) a catalytically inactive guided-nuclease;
(b) at least one guide nucleic acid, wherein the at least one guide nucleic acid forms a complex with the catalytically inactive guided-nuclease, and wherein the at least one guide nucleic acid hybridizes with the target nucleic acid molecule; and
(c) at least one mutagen,
wherein the target nucleic acid molecule comprises a protospacer adjacent motif (PAM) site, and wherein the mutation rate in the targeted region of the nucleic acid molecule is increased compared to an untargeted nucleic acid molecule.

4. A method of increasing allelic diversity in a target region of a nucleic acid molecule within a genome of a plant, comprising providing to the plant:
(a) a catalytically inactive guided-nuclease or a nucleic acid encoding the catalytically inactive guided-nuclease;
(b) at least one guide nucleic acid or a nucleic acid encoding the at least one guide nucleic acid, wherein the at least one guide nucleic acid forms a complex with the catalytically inactive guided-nuclease, and wherein the at least one guide nucleic acid hybridizes with the nucleic acid molecule; and
(c) at least one mutagen,
wherein the nucleic acid comprises a protospacer adjacent motif (PAM) site adjacent to the targeted region, and wherein allelic diversity of the target region of the nucleic acid molecule is increased.

5. (canceled)

6. (canceled)

7. The method of claim 1, wherein the method or kit further comprises (c) at least one guide nucleic acid or a nucleic acid encoding the at least one guide nucleic acid, wherein the at least one guide nucleic acid forms a complex with the catalytically inactive guided-nuclease, and wherein the at least one guide nucleic acid hybridizes with the target nucleic acid molecule.

8. The method of claim 1, wherein the catalytically inactive guided-nuclease comprises a DNA-binding domain.

9. (canceled)

10. The method of claim 1, wherein the target nucleic acid molecule is located in a cell.

11. (canceled)

12. The method of claim 10, wherein the cell is an Escherichia coli cell.

13. The method of claim 10, wherein the cell is a plant cell or an animal cell.

14. The method of claim 13, wherein said plant cell is selected from the group consisting of: a corn cell, a cotton cell, a canola cell, a soybean cell, a rice cell, a tomato cell, a wheat cell, an alfalfa cell, a sorghum cell, an Arabidopsis cell, a cucumber cell, a potato cell, and an algae cell.

15. The method of claim 10, wherein (i) the catalytically inactive guided-nuclease, or a nucleic acid encoding the catalytically inactive guided-nuclease; or (ii) the at least one guide nucleic acid, or a nucleic acid encoding the at least one guide nucleic acid; or (iii) both (i) and (ii); are provided to the cell via a method selected from the group consisting of: Agrobacterium-mediated transformation, polyethylene glycol-mediated transformation, biolistic transformation, liposome-mediated transfection, viral transduction, the use of one or more delivery particles, microinjection, and electroporation.

16. The method of claim 3, wherein the catalytically inactive guided-nuclease and the at least one guide RNA are provided as a ribonucleoprotein.

17. The method of claim 16, wherein the ribonucleoprotein is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

18. (canceled)

19. The method of claim 15, wherein (i) the catalytically inactive guided-nuclease, or nucleic acid encoding the catalytically inactive guided-nuclease, or (ii) the at least one guide nucleic acid, or a nucleic acid encoding the at least one guide nucleic acid are provided to the cell in vivo, in vitro, or ex vivo.

20. The method of claim 17, wherein the ribonucleoprotein is provided to the cell in vivo, in vitro, or ex vivo.

21. (canceled)

22. The method of claim 1, wherein the at least one mutagen is a chemical mutagen is selected from the group consisting of ethyl methanesulfonate, methyl methanesulfonate, diethylsulphonate, dimethyl sulfate, dimethyl sulfoxide, diethylnitrosamine, N-nitroso-N-methylurea, N-methyl-N-nitrosourea, N-nitroso-N-diethyl urea, arsenic, colchicine, ethyleneimine, nitrosomethylurea, nitrosoguanidine, nitrous acid, hydroxylamine, ethyleneoxide, diepoxybutane, sodium azide, maleic hydrazide, cyclophosphamide, diazoacetylbutan, Datura extract, bromodeoxyuridine, and beryllium oxide.

23. (canceled)

24. The method of claim 1, wherein the catalytically inactive guided-nuclease is a catalytically inactive CRISPR nuclease.

25. The method of claim 24, wherein the catalytically inactive CRISPR nuclease is selected from the group consisting of a catalytically inactive Cas9, a catalytically inactive Cpf1, a catalytically inactive CasX, a catalytically inactive CasY, a catalytically inactive C2c2, a catalytically inactive Cas1, a catalytically inactive Cas1B, a catalytically inactive Cas2, a catalytically inactive Cas3, a catalytically inactive Cas4, a catalytically inactive Cas5, a catalytically inactive Cas6, a catalytically inactive Cas7, a catalytically inactive Cas8, a catalytically inactive Cas10, a catalytically inactive Csy1, a catalytically inactive Csy2, a catalytically inactive Csy3, a catalytically inactive Cse1, a catalytically inactive Cse2, a catalytically inactive Csc1, a catalytically inactive Csc2, a catalytically inactive Csa5, a catalytically inactive Csn2, a catalytically inactive Csm1, a catalytically inactive Csm2, a catalytically inactive Csm3, a catalytically inactive Csm4, a catalytically inactive Csm5, a catalytically inactive Csm6, a catalytically inactive Cmr1, a catalytically inactive Cmr3, a catalytically inactive Cmr4, a catalytically inactive Cmr5, a catalytically inactive Cmr6, a catalytically inactive Csb1, a catalytically inactive Csb2, a catalytically inactive Csb3, a catalytically inactive Csx17, a catalytically inactive Csx14, a catalytically inactive Csx10, a catalytically inactive Csx16, a catalytically inactive CsaX, a catalytically inactive Csx3, a catalytically inactive Csx1, a catalytically inactive Csx15, a catalytically inactive Csf1, a catalytically inactive Csf2, a catalytically inactive Csf3, and a catalytically inactive Csf4.

26.-29. (canceled)

30. The method of claim 3, wherein the at least one guide nucleic acid comprises a single-molecule guide.

31. The method of claim 3, wherein the at least one guide nucleic acid comprises at least 80% complementarity to a target region of the target nucleic acid molecule.

32. The method of claim 1, wherein the targeted modification is selected from the group consisting of a substitution, an insertion, and a deletion.

33.-40. (canceled)
